(19) United States
(12) Patent Application Publication
(10) Pub. No.: US 2025/0302624 A1
(43) Pub. Date: Oct. 2, 2025

(54) LEAFLET AUGMENTER

(71) Applicant: Edwards Lifesciences Innovation (Israel) Ltd., Caesarea (IL)

(72) Inventors: Or Cohen, Modi'in (IL); Yaron Herman, Givat Ada (IL); Ido Halabi, Givatayim (IL); David M. Taylor, Lake Forest, CA (US); Daniela Shabodyash, Atlit (IL); Natan Anatoly Bukhdruker, Tirat Carmel (IL); Lior Harush, Herzliya (IL); Valentina Ruban, Holon (IL); Carmel Peleg, Pardes Hana-Karkur (IL); Anna Khodos, Harish (IL); Bar Eytan-Vaisman, Gedera (IL); Sergio Delgado, Irvine, CA (US); Bingquan Su, Irvine, CA (US); Haim Brauon, Beit Dagan (IL); Gal Aviram, Kiryat Tiv'on (IL); Tal Sheps, Givat Shmuel (IL)

(21) Appl. No.: 19/231,022

(22) Filed: Jun. 6, 2025

Related U.S. Application Data
(63) Continuation of application No. PCT/IB2023/062348, filed on Dec. 7, 2023.
(60) Provisional application No. 63/386,886, filed on Dec. 9, 2022.
(60) Provisional application No. 63/505,400, filed on May 31, 2023.
(60) Provisional application No. 63/508,443, filed on Jun. 15, 2023.
(60) Provisional application No. 63/582,494, filed on Sep. 13, 2023.

(51) Int. Cl. A61F 2/24

(52) U.S. Cl. CPC A61F 2/2463; A61F 2/2418; A61F 2220/0025; A61F 2230/0067; A61F 2250/0003; A61F 2250/0007; A61F 2250/0018

(57) ABSTRACT

Apparatus, usable with a valve disposed between an atrium and a ventricle of a heart, the valve having a first leaflet (S) and at least one opposing leaflet (A,P). The apparatus comprises an augmenter (700) that comprises a flexible pouch (710) and an anchor (780). The pouch has an external surface, an interior, an outside, and a downstream opening that provides fluid communication between the interior and the outside of the pouch. The anchor is coupled to the pouch, and configured to anchor the pouch to the lip of the first leaflet by the anchor being anchored to the first leaflet. The anchoring is such that the downstream opening opens into the ventricle. Responsively to ventricular systole, the pouch inflates via the downstream opening, and the external surface coapts against the opposing leaflet(s).

### CROSS-REFERENCES TO RELATED APPLICATIONS

This application is a continuation of International Patent Application No. PCT/IB2023/062348, filed Dec. 7, 2023, which claims the benefit of: U.S. Patent Application No. 63/386,886, filed Dec. 9, 2022; U.S. Patent Application No. 63/505,400, filed May 31, 2023; U.S. Patent Application No. 63/508,443, filed Jun. 15, 2023; and U.S. Patent Application No. 63/582,494, filed Sep. 13, 2023, the entire disclosures all of which are incorporated by reference for all purposes.

This application is related to International Patent Application No. PCT/IB2022/062188 to Ruban et al., titled “Leaflet Augmentor,” filed Dec. 14, 2022, which published as WO 2023/119065, the entire disclosure which is incorporated by reference for all purposes.

### BACKGROUND

Cardiac valves are subject to damage from a variety of causes. Damaged valves may result in regurgitation of blood from the ventricle to the atrium during ventricular systole, eventually causing heart failure. Devices designed to prevent backflow through the damaged valve could be helpful.

### SUMMARY

This summary is meant to provide some examples and is not intended to be limiting of the scope of the disclosure in any way. For example, any feature included in an example of this summary is not required by the claims, unless the claims explicitly recite the features. Also, the features, components, steps, concepts, etc. described in examples in this summary and elsewhere in this disclosure can be combined in a variety of ways. Various features and steps as described elsewhere in this disclosure can be included in the examples summarized here.

Example implementations described herewithin are directed towards systems, apparatuses, devices, and methods to reduce regurgitation through a real or simulated heart valve by improving coaptation between leaflets of the valve, e.g., by diminishing leaflet flail, prolapse, or abnormal leaflet motion. For example, leaflet augmenters (alternatively spelled augmentors) are described.

In accordance with some implementations, a system and/or an apparatus, (which can be used with a valve of a heart, e.g., useable with a valve disposed between an atrium and a ventricle of a real or simulated heart, the valve having a first leaflet and an opposing leaflet, the first leaflet having a lip) includes an augmenter that includes: a flexible pouch, having an external surface, an interior, and a downstream opening that provide fluid communication between the interior and an outside of the pouch; and/or an anchor, coupled to the pouch, and configured to anchor the pouch to the lip of the first leaflet by the anchor being anchored to the first leaflet, such that the downstream opening opens into the ventricle, and/or responsively to ventricular systole: the pouch inflates via the downstream opening; and/or the external surface coapts against the opposing leaflet.

For some implementations, the pouch is rectangular. For some implementations, the pouch is mitre-shaped. For some implementations, the pouch is conical. For some implementations, the pouch is pillow-shaped. For some implementations, the pouch is shaped as a rounded square. For some implementations, the pouch is globular. For some implementations, the pouch is pyramidal. For some implementations, the pouch is tetrahedral.

For some implementations, the augmenter is sterile.

For some implementations, the anchor includes a clip. For some implementations, the anchor includes a pair of clips.

For some implementations, the first leaflet has a root attached to an annulus of the valve; and/or the anchor is configured to be anchored to the first leaflet closer to the lip than to the root.

For some implementations, the augmenter includes a film from which the pouch is formed. For some implementations, the augmenter includes a polymer from which the pouch is formed.

For some implementations, the anchor is configured to anchor the pouch to the lip of the first leaflet such that the pouch deflates responsively to ventricular diastole.

For some implementations, the opposing leaflet is a first opposing leaflet, the valve is a tricuspid valve having a second opposing leaflet, and/or the anchor is configured to anchor the pouch to the lip of the first leaflet such that responsively to ventricular systole, the pouch inflates via the downstream opening, such that the external surface coapts against both the first opposing leaflet and the second opposing leaflet.

For some implementations, the anchor is configured to anchor the pouch to the lip of the first leaflet such that, responsively to ventricular systole, the pouch inflates via the downstream opening, such that at least part of the pouch interposes between the first opposing leaflet and the second opposing leaflet. For some implementations, the anchor is selected from: tissue-piercing elements, T-bars, threads, sutures, pins, clips, and locking elements.

For some implementations, the system further includes a stem that couples the pouch to the anchor by extending from the anchor, outside of the pouch toward the downstream opening, under a rim of the downstream opening, thereby defining a bight of the stem, and/or from the bight, through the interior to an upstream apex of the pouch, thereby defining a mast of the augmenter.

For some implementations, the augmenter includes an adjustment mechanism that is actuatable to adjust positioning between the anchor and the pouch. For some implementations, the anchor is coupled to the pouch in a manner that facilitates adjustment of positioning between the anchor and the pouch. For some implementations, the augmenter is configured to facilitate adjustment of positioning between the anchor and the pouch while the pouch remains anchored to the lip by the anchor, in a manner that changes a proportion of the pouch that is disposed within the atrium during ventricular systole.

For some implementations, the system further includes a stem via which the pouch is adjustably coupled to the anchor.

For some implementations, the valve is a tricuspid valve, the first leaflet is a septal leaflet of the tricuspid valve; and/or the anchor is configured to anchor the pouch to the lip of the septal leaflet by being anchored to the septal leaflet.

For some implementations, the augmenter further includes an adjustable strap coupled to the pouch such that adjusting a length of the strap adjusts an internal dimension of the pouch.

For some implementations, the adjustable strap spans the interior of the pouch. For some implementations, the adjustable strap spans the downstream opening. For some implementations, the adjustable strap lies parallel to the internal dimension.

For some implementations, the opposing leaflet is a first opposing leaflet, the valve is a tricuspid valve having a second opposing leaflet, and/or the adjustable strap is configured to reshape the external surface to increase coaptation, during ventricular systole, between the external surface and the first and second opposing leaflets.

For some implementations, the adjustable strap includes a fabric. For some implementations, the adjustable strap is an adjustable strap selected from: a belt, a cord, a line, a thread, a braid, a thong, a tape, a tie, a cable, a wire, an elastic, and a ligature. For some implementations, the adjustable strap includes a belt with a buckle. For some implementations, the adjustable strap includes a ratchet. For some implementations, the adjustable strap includes a locking mechanism. For some implementations, the adjustable strap includes a hook and loop self-fastener.

For some implementations, decreasing a length of the adjustable strap limits inflation of the pouch.

For some implementations, the adjustable strap is a first adjustable strap, and the augmenter further includes a second adjustable strap.

For some implementations, the pouch has a longitudinal plane, the first adjustable strap and the second adjustable strap being disposed on opposite sides of the longitudinal plane from each other. For some implementations, the first adjustable strap lies parallel with the longitudinal plane. For some implementations, the first adjustable strap lies parallel with the second adjustable strap.

For some implementations, the longitudinal plane bisects the downstream opening. For some implementations, the anchor is disposed on the longitudinal plane.

For some implementations, the pouch has a transverse plane, the first adjustable strap and the second adjustable strap being disposed on opposite sides of the transverse plane from each other. For some implementations, the first adjustable strap lies parallel with the transverse plane. For some implementations, the first adjustable strap lies parallel with the second adjustable strap.

For some implementations, the first adjustable strap is disposed on an upstream side of the transverse plane, and/or the second adjustable strap and the downstream opening are disposed on a downstream side of the transverse plane. For some implementations, the anchor is disposed on the transverse plane.

For some implementations, the external surface defines a central opening that provides fluid communication between the interior and the outside of the pouch.

For some implementations, the augmenter includes a fabric from which the pouch is formed. For some implementations, the pouch includes and/or is formed by three-dimensional weaving of the fabric. For some implementations, the pouch includes and/or is formed by three-dimensional knitting of the fabric.

For some implementations, the augmenter includes a polymer from which the pouch is formed. For some implementations, the pouch includes and/or is formed by three-dimensional weaving of the polymer. For some implementations, the pouch includes and/or is formed by three-dimensional knitting of the polymer.

For some implementations, the augmenter has a ceiling at an opposite end of the augmenter from the downstream opening, and/or the anchor is configured to anchor the augmenter to the lip of the first leaflet such that the downstream opening is downstream of the ceiling. For some implementations, the anchor is configured to anchor the augmenter to the lip of the first leaflet such that the ceiling is disposed in the atrium during ventricular systole.

For some implementations, the augmenter further includes a frame to which the pouch and the anchor are coupled; and/or a stabilizer, coupled to the frame, extending away from the frame to a projection of the stabilizer, the stabilizer being configured to be positioned with the projection at a site in the heart in a manner that resists upstream pivoting of the pouch.

For some implementations, the stabilizer includes a resilient wire, and/or the anchor is configured to anchor the pouch to the lip of the first leaflet such that the resilient wire extends in an arc upstream of the pouch and presses the projection into a commissure of the valve. For some implementations, the resilient wire defines a bowed spring.

For some implementations, the projection is shaped to hook against a downstream surface of the commissure.

For some implementations, the frame defines a mast, the projection is configured to be driven into heart tissue upstream of the valve, and/or the stabilizer includes a tether that tethers the mast to the projection such that the tether becomes tensioned during ventricular systole.

For some implementations, the projection includes a tissue anchor. For some implementations, the projection includes a foil that has a shape and an angle of attack that generates lift in a manner that resists the upstream pivoting.

For some implementations, the tether, by utilizing hemodynamic forces, traverses a pulmonary valve of the heart such that the foil is disposed within a pulmonary artery of the heart.

For some implementations, the tether, by utilizing hemodynamic forces, is disposed within the ventricle.

For some implementations, the ventricle has a septal wall, the stabilizer includes a tether, and/or the projection includes a tissue anchor configured to be anchored to the septal wall in a manner that limits atrial pivoting of the pouch during ventricular contraction. For some implementations, the tissue anchor is configured to be anchored to the septal wall by traversing the septal wall.

For some implementations, the projection includes a resilient spring, and/or the anchor is configured to anchor the pouch to the lip of the first leaflet in a manner that places the projection against a wall of the atrium.

For some implementations, the stabilizer includes a bowed spring, and/or the augmenter is configured to be positioned such that the bowed spring presses against a wall of the ventricle.

For some implementations, the bowed spring defines the projection, and the stabilizer further includes a tether that tethers the bowed spring to the frame. For some implementations, the bowed spring is shaped to extend circumferentially around the ventricle and to press radially outward against the wall of the ventricle.

For some implementations, the stabilizer includes a tether that tethers the projection to the frame. For some implementations, the stabilizer includes a resilient wire that couples the projection to the frame.

For some implementations, the projection includes an expandable stent, configured to be secured within a cardiovascular lumen by being expanded within the cardiovascular lumen.

For some implementations, the projection is configured to be secured within a superior vena cava. For some implementations, the projection is configured to be secured within an inferior vena cava. For some implementations, the projection is configured to be secured within a pulmonary artery. For some implementations, the projection is configured to be secured within a coronary sinus.

For some implementations, the first leaflet is attached to chordae tendineae of the heart; and/or the augmenter further includes a stabilizer, coupled to the pouch, and configured to be looped behind chordae tendineae of the first leaflet in a manner that resists upstream pivoting of the pouch.

For some implementations, the augmenter further includes a frame to which the pouch and the anchor are coupled, the stabilizer being coupled to the pouch by being coupled to the frame.

For some implementations, the stabilizer includes a rod, and is configured to be looped behind the chordae tendineae of the first leaflet in a manner that positions the rod behind the chordae tendineae.

For some implementations, the stabilizer includes a wire that is coupled to the rod and that has a length that is adjustable in a manner that adjusts the resisting of the upstream pivoting of the pouch.

For some implementations, the pouch has a transverse plane disposed between a proximal end of the pouch and the downstream opening, and the stabilizer is configured to be looped behind the chordae tendineae of the first leaflet in a manner that positions the rod behind the chordae tendineae, parallel to the transverse plane.

For some implementations, the stabilizer is configured to be looped behind the chordae tendineae of the first leaflet such that the rod when positioned behind the chordae tendineae, extends laterally beyond the pouch parallel to the transverse plane.

For some implementations, the pouch is shaped to define a vent at an upstream region of the pouch, the vent configured to allow limited retrograde blood flow therethrough.

For some implementations, the vent is one of multiple vents at the proximal end of the pouch. For some implementations, the multiple vents are disposed on an opposite side of the pouch to the anchor.

For some implementations, the pouch is conical and defines an apex, the vent being disposed at the apex. For some implementations, the vent includes a grommet that delimits the vent.

For some implementations, the vent includes a check valve that is oriented to allow the limited retrograde blood flow through the vent and to inhibit antegrade blood flow through the vent. For some implementations, the check valve is a duckbill valve. For some implementations, the check valve is a reed valve. For some implementations, the check valve is a diaphragm valve.

For some implementations, the apparatus further includes a resilient wire, coupled to an upstream part of the pouch in a manner that squeezes blood out of the upstream part of the pouch during diastole.

For some implementations, the valve is a tricuspid valve having the first leaflet, a second leaflet, and the opposing leaflet. The anchor is a first anchor, and is coupled to the pouch at a first face of the pouch. The augmenter further includes a second anchor, coupled to the pouch at a second face of the pouch, the external surface being an external surface of a third face of the pouch; and/or the first and second anchors are configured to anchor the first and second faces, respectively, to the first and second leaflets, respectively, such that, responsively to ventricular systole, the external surface of the third face of the pouch coapts against the opposing leaflet.

For some implementations, the apparatus further includes a frame internal to the pouch to which the first and second anchors are fixedly coupled. For some implementations, the pouch is dimensioned and coupled to the frame, such that, upon anchoring of the first and second faces to the first and second leaflets by the first and second anchors, the pouch projects into the ventricle.

For some implementations, the pouch is dimensioned and coupled to the frame, such that, upon anchoring of the first and second faces to the first and second leaflets by the first and second anchors, the pouch projects into the atrium.

For some implementations, the system further includes a frame to which the pouch and the anchor are fixedly coupled, the frame including a hinge; and/or a tube slidably coupled to the hinge such that sliding the tube over the hinge inactivates the hinge.

For some implementations, the hinge is configured to confer flexibility on a segment of the frame, and sliding the tube over the hinge stiffens the segment. For some implementations, the tube is configured to lock into place after sliding over the hinge.

For some implementations, the augmenter includes a frame to which the anchor is coupled, the anchor including an upstream clasp element and a downstream clasp element hingedly coupled to the frame.

For some implementations, the apparatus includes a delivery tool configured to transluminally advance the augmenter to the valve, position the downstream clasp element at a downstream side of the first leaflet by passing the downstream clasp element between chordae tendineae of the first leaflet, actuate, at the downstream side of the first leaflet, the downstream clasp element to expand laterally, and/or anchor the anchor to the first leaflet by clasping the first leaflet between (i) the laterally-expanded downstream clasp element against the downstream side of the first leaflet, and (ii) the upstream clasp element against an upstream side of the first leaflet.

For some implementations, the downstream clasp element is restrained laterally by the delivery tool until deployment, and/or the delivery tool is configured to actuate the downstream clasp element by releasing the downstream clasp element to expand.

For some implementations, the delivery tool is configured to actuate the downstream clasp element by pulling on the downstream clasp element. For some implementations, the delivery tool is configured to actuate the downstream clasp element by pushing on the downstream clasp element. For some implementations, the delivery tool is configured to actuate the downstream clasp element by unsheathing the downstream clasp element.

For some implementations, the apparatus further includes a stabilizer, coupled to the anchor, extending away from the anchor and the pouch, and shaped such that, when the anchor is anchored to the first leaflet, the stabilizer rests on a tissue surface of the atrium.

For some implementations, the valve is a tricuspid valve; the first leaflet is a septal leaflet of the tricuspid valve; and/or the stabilizer includes two curved arms configured to extend in opposite directions from a point in the atrium above the anchoring of the anchor to the first leaflet, in a manner that the curved arms are configured to rest on a septal wall of the atrium above an atrioventricular node of the heart.

For some implementations, the stabilizer includes a loop coupled to a rod, the rod coupled to the anchor and configured to be positioned over the first leaflet, and the loop configured to rest on an annulus of the valve.

For some implementations, the apparatus further includes a stabilizer spring configured to couple the rod to the stem, and/or facilitate lateral flexibility of the pouch.

For some implementations, the pouch is shaped to have a base at which the downstream opening is located, and an apical region that that includes an apex, the pouch tapering from the base toward the apex.

For some implementations, the pouch is conical. For some implementations, the downstream opening is substantially elliptical. For some implementations, the pouch is tetrahedral. For some implementations, the downstream opening is substantially triangular.

For some implementations, the apparatus further includes a set of supports diverging from the apical region toward the base; and/or coupled to the pouch in a manner that shapes the pouch to be tetrahedral.

For some implementations, the set of supports is coupled to the anchor.

For some implementations, the augmenter further includes a leg coupled to, and protruding away from, at least one of the supports such that the leg tucks under the opposing leaflet. For instance, responsively to ventricular systole, as the external surface coapts against the opposing leaflet, the leg can tuck under the opposing leaflet.

For some implementations, the opposing leaflet is a first opposing leaflet of a pair of opposing leaflets, and/or the leg is shaped to define a pair of grasps that diverge from each other. For instance, responsively to ventricular systole, as the external surface coapts against the opposing leaflet each of the grasps of the leg tucks under a respective opposing leaflet of the pair of opposing leaflets. For some implementations, a length of the leg is adjustable.

For some implementations, the opposing leaflet is a first opposing leaflet of a pair of opposing leaflets, and/or the leg is shaped to define a pair of grasps that diverge from each other. For instance in some implementations, responsively to ventricular systole, as the external surface coapts against the opposing leaflet the grasps of the leg engage a commissure at which the pair of opposing leaflets meet.

For some implementations, the augmenter further includes a leg protruding externally from the pouch and away from the downstream opening. For instance, in some implementations, responsively to ventricular systole, as the external surface coapts against the opposing leaflet, the leg tucks under the opposing leaflet.

For some implementations, the leg is a first leg, and the apparatus includes multiple legs. For some implementations, at least some of the legs are covered by a growth-promoting material.

For some implementations, the opposing leaflet is a first opposing leaflet, the valve including a pair of opposing leaflets, and/or each of the multiple legs is configured to tuck under one of the pair of opposing leaflets.

For some implementations, the augmenter includes a frame that is coupled to the pouch along at least part of a perimeter of the downstream opening. In some implementations, the multiple legs are defined by the frame and are distributed around at least the part of the perimeter.

For some implementations, the apparatus further includes a support that is coupled to the anchor, and that forks to define diverging limbs that are coupled to the frame on opposing sides of the perimeter. For some implementations, the frame is annular and circumscribes the perimeter of the downstream opening.

For some implementations, the pouch is configured with a vent at the apex. For some implementations, the vent is one of multiple vents at the apical region.

For some implementations, the vent includes a grommet that delimits the vent. For some implementations, the vent includes a check valve that is oriented to allow the limited retrograde blood flow through the vent and to inhibit antegrade blood flow through the vent. For some implementations, the check valve is a duckbill valve. For some implementations, the check valve is a reed valve. For some implementations, the check valve is a diaphragm valve.

For some implementations, the flexible pouch can comprise and/or is formed from a fabric, and/or the pouch includes at least one wire that has a stiffness greater than that of the fabric, and that extends, in association with the fabric, from an apical vent toward the downstream opening.

For some implementations, the at least one wire includes at least three wires, distributed circumferentially around the pouch, such that the at least three wires deflect away from each other as the pouch inflates.

For some implementations, the apparatus further includes a stem that couples the pouch to the anchor in a manner that enables adjustment of a position of the pouch with respect to the anchor.

For some implementations, the augmenter is configured such that, when the anchor is anchored to the first leaflet, progressive adjustment of the position of the pouch atrial-ward positions progressively wider regions of the pouch between the first leaflet and the opposing leaflet.

For some implementations, when the anchor is anchored to the lip of the first leaflet, progressive adjustment of the position of the pouch ventricle-ward positions progressively narrower regions of the pouch between the first leaflet and the opposing leaflet.

For some implementations, the stem is axially fixed with respect to the pouch, and threadedly coupled to the anchor.

For some implementations, the stem is axially fixed with respect to the anchor, and threadedly coupled to the pouch.

For some implementations, the augmenter further includes a sleeve, fixed to the anchor, and/or the stem is fixed to the pouch, and couples the pouch to the anchor by being disposed through the sleeve.

For some implementations, the augmenter further includes a sleeve having internal threads, and the stem is coupled to the pouch via a rotary bearing at either end of the stem; and/or the anchor by being disposed through the sleeve, the stem having external threads such that rotating the stem within the sleeve adjusts a position of the pouch along the sleeve.

For some implementations, the augmenter further includes a sleeve, and the stem is coupled to the anchor by being disposed through the sleeve in a manner that defines a rotary bearing, and/or the pouch via at least one threaded nut, such that rotating the stem within the at least one threaded nut adjusts a position of the pouch along the stem.

For some implementations, the stem is coupled to the pouch at the apical region of the pouch, and/or the augmenter further includes a threaded nut, at the apical region of the pouch, configured such that rotating the threaded nut adjusts a position of the pouch along the stem.

For some implementations, the augmenter further includes an adjustment spring, arranged with respect to the threaded nut such that rotating the threaded nut in a first rotational direction strains the spring, and rotating the threaded nut in an opposite rotational direction relaxes the spring.

For some implementations, the adjustment spring is disposed coaxially around the stem within the interior of the pouch.

For some implementations, the stem extends from the anchor toward the downstream opening, under a rim of the downstream opening, thereby defining a bight of the stem, and/or from the bight, through the interior to the apical region, thereby defining a mast of the augmenter, the mast being coupled to the pouch at the apical region.

For some implementations, the augmenter further includes a sleeve, fixed to the anchor, and/or the stem couples the pouch to the anchor by being disposed through the sleeve, and enables adjustment of the position of the pouch with respect to the anchor via sliding of the stem through the sleeve.

For some implementations, the stem includes a rod that has ends that are fixed to the pouch, and a midsection, between the ends; and/or progressively sliding the midsection through the sleeve in an atrial-ward direction positions progressively wider regions of the pouch between the first leaflet and the opposing leaflet.

For some implementations, the stem includes a rod that has ends that are fixed to the pouch, and a midsection, between the ends; and/or progressively sliding the midsection through the sleeve in a ventricle-ward direction positions progressively narrower regions of the pouch between the first leaflet and the opposing leaflet.

For some implementations, the stem has a first region that is slidable through the sleeve, and/or a second region fixed to the apical region of the pouch, the second region being stiffer than the first region.

For some implementations, the augmenter is configured such that, when the anchor is anchored to the first leaflet, progressively sliding the first region through the sleeve in an atrial-ward direction positions progressively narrower regions of the pouch between the first leaflet and the opposing leaflet.

For some implementations, the augmenter is configured such that, when the anchor is anchored to the first leaflet, progressively sliding the first region through the sleeve in a ventricle-ward direction positions progressively wider regions of the pouch between the first leaflet and the opposing leaflet.

For some implementations, the augmenter is configured such that, when the anchor is anchored to the first leaflet, sliding the first region through the sleeve in an atrial-ward direction moves the pouch in a ventricle-ward direction.

For some implementations, the augmenter is configured such that, when the anchor is anchored to the first leaflet, sliding the first region through the sleeve in a ventricle-ward direction moves the pouch in an atrial-ward direction.

For some implementations, the augmenter further includes a threaded nut disposed at an atrial region of the sleeve, configured such that rotating the threaded nut adjusts a position of the stem along the sleeve.

For some implementations, the stem: has a first part that extends from the anchor toward the downstream opening, has a second part that defines a mast that extends through the interior to the apical region, the mast being coupled to the pouch at the apical region, and/or includes an adaptive joint that couples the first part to the second part in a manner that facilitates passive reshaping of the stem responsively to intracardial forces.

For some implementations, the adaptive joint includes a ball-and-socket joint. For some implementations, the adaptive joint includes a floating joint configured to facilitate lateral adjustability of the pouch. For some implementations, the adaptive joint includes a hinge.

For some implementations, the stem defines a mast that extends through the interior to the apical region, the mast being coupled to the pouch at the apical region via a ratchet mechanism that enables adjustment of the position by ratcheting the mast through the apical region.

For some implementations, the ratchet mechanism includes a set of teeth arranged along the mast, and at least one pawl at the apical region, enabling adjustment of the position by ratcheting the teeth through the apical region.

For some implementations, when the augmenter is anchored to the lip of the first leaflet, ratcheting the mast through the apical region moves the pouch atrial-ward. For some implementations, when the augmenter is anchored to the lip of the first leaflet, ratcheting the mast though the apical region moves the pouch ventricular-ward.

In some implementations, a leaflet augmenter comprises two articulatably-coupled panels. In some implementations, the first panel is anchorable to a first valve leaflet by anchors, e.g., tissue-piercing elements, T-bars, threads, sutures, pins, clips, or locking elements. In some implementations, the second panel is expandable or collapsible via the articulatable coupling, such that the second panel moves away from or toward the first panel during phases of the cardiac contraction cycle. That is, during ventricular diastole, the second panel can be configured to move toward the first panel in response to blood flow through the valve, whereas during ventricular systole, the second panel can be configured to move away from the first panel, e.g., in response to increased intraventricular pressure.

In some implementations, as the valve closes, the second panel coapts with a second valve leaflet that, prior to implantation of the augmenter, did not coapt properly with the first leaflet.

In some implementations, a flexible connector is disposed between the first panel and the second panel, in a manner that limits expansion of the augmenter by limiting the angular extent of an opening between the first panel and the second panel. In some implementations, the flexible connector can connect lips of the first and second panels. In some implementations, the flexible connector can connect lateral edges of the first and second panels.

In some implementations, the connector can comprise at least one wire, string, or thread.

In some implementations, the connector comprises a flexible third panel. The third panel can comprise a permeable net, e.g., an open-meshed fabric, a web of interwoven threads, or other substantially open structure. In some implementations, the third panel has an accordion fold.

In some implementations in which the connector comprises a third panel, the augmenter further comprises a flexible fourth panel, e.g., with each of the third and fourth panels connecting respective lateral edges of the first and second panels. Such an arrangement can stabilize the augmenter and limit the extent of opening between the first panel and the second panel.

The connector can include a cell-growth-inhibiting substance. The connector can include a cell-growth-promoting substance.

The panels can have a substantially rectangular shape or a substantially elliptical shape or can have a different suitable shape.

The panels can comprise a flexible fabric. In some implementations, the fabric can have tissue growth-promoting or tissue growth-inhibiting properties. In some implementations, one side of each panel can comprise fabric having different properties than the fabric on the opposite side of the panel, and/or the fabric of one panel can have different properties than the fabric of the other panel.

In some implementations, the augmenter comprises a frame, which can be elastic. In some implementations in which the panels comprise a fabric, the frame can support the fabric.

In some implementations, each panel can comprise a two-ply fabric compartment into which the frame is inserted.

In some implementations, the elastic frame can be pretensioned within the fabric frame.

In some implementations in which the augmenter comprises a frame, in addition to or alternatively to the augmenter comprising a flexible connector that limits expansion of the augmenter, the frame can itself limit expansion of the augmenter. In some implementations, the articulable coupling can also be part of a frame or frames disposed circumferentially within one or both panels, as described herein below.

In some implementations, the augmenter can be biased toward the first panel and the second panel having a predetermined angular disposition therebetween. In some implementations, the coupling between the first and second panels can contribute to limiting the expansion of the augmenter.

In some implementations, the coupling can be used to bias the augmenter toward the two panels being in either a collapsed state or an expanded state.

In some implementations, the stiffness of the articulatable coupling, e.g., a hinge, is biased to respond to blood flow through the valve such the pressure of blood passing from an atrium to a ventricle of a heart during diastole tends to collapse the second panel against the first panel.

In some implementations, the augmenter can be biased toward an expanded or collapsed state by means other than the articulatable coupling itself. For example, elasticity and/or shape memory of the frame can provide such biasing.

In some implementations, a discrete spring (e.g., a torsion spring, a leaf spring, a cantilever spring, or an arc spring) can provide such biasing, e.g., can provide the augmenter with a clothespin-like characteristic (e.g., biased either open or closed).

In some implementations, a leaflet augmenter comprises three panels disposed around a central axis and diverging from each other. In some implementations, the panels can be arranged at equal angles, e.g., 120 degrees between each pair. In some implementations, the panels can be arranged such that a first and a second panel are disposed at less than 120 degrees from each other, and the third panel at more than 120 degrees from each of the other panels.

In some implementations, a first of the three panels is anchorable via an anchor to a first valve leaflet.

In some implementations, the augmenter can be configured such that, during systole, one or both of the second and third panels coapts with a second valve leaflet that, prior to implantation of the augmenter, did not coapt properly with the first leaflet.

In some implementations, a leaflet augmenter comprises a panel comprising a flexible sheet, a frame, and an anchor coupled to the frame and configured to anchor the augmenter to a lip of a first valve leaflet.

In some implementations, the flexible sheet can have a first surface that faces the frame and a second surface that faces away from the frame, and can be coupled to the frame at specific points, e.g., in a manner that provides a billow zone at which the sheet is unattached to the frame. In some implementations, the billow zone may thus facilitate the panel billowing away from the frame responsively to fluid pressure at the first surface.

In some implementations, the augmenter can further define a second billow zone that facilitates the sheet, at the second billow zone, billowing away from the frame in a second direction responsively to the fluid pressure, the second direction being opposite to the first direction.

In some implementations, a leaflet augmenter comprises a unitary skeleton, cut from a sheet of shape-memory alloy to define a frame-region and an anchor-region.

In some implementations, in a first heat-setting procedure, the anchor-region can undergo heat-setting to define an anchor. In some implementations, in a second heat-setting procedure, the frame-region can undergo heat-setting to define a frame.

In some implementations, the augmenter can further define a sheet, attached to the frame in a manner that defines a panel.

In some implementations, upon implantation, the anchor is configured to anchor the augmenter to the first leaflet such that the panel coapts with the second leaflet during systole.

In some implementations, the heat setting steps can be performed using molds or jigs specific for the desired shape of the anchor and the frame, respectively.

In accordance with some implementations, a system and/or an apparatus (e.g., usable and/or for use with a valve of a heart, the valve having a first leaflet and a second leaflet, which first leaflet can include a lip) includes an augmenter (which can be the same as or similar to other augmenters elsewhere herein) that includes: a frame, and an anchor, coupled to the frame.

In some implementations, the anchor is configured to anchor the augmenter to the lip of the first leaflet. The anchor can be the same as or similar to any of the anchors described here or elsewhere herein.

In some implementations, the augmenter comprises a panel including a flexible sheet having a first surface that faces the frame and a second surface that faces away from the frame.

In some implementations, the sheet is coupled to the frame in a manner that provides a billow zone at which the flexible sheet is unattached to the frame, thereby facilitating the panel billowing away from the frame responsively to fluid pressure at the first surface.

In some implementations, the augmenter is sterile.

In some implementations, the billow zone is a first billow zone, the first billow zone facilitates the flexible sheet, at the first billow zone, billowing away from the frame in a first direction responsively to the fluid pressure. In some implementations, the flexible sheet is coupled to the frame so as to further define at least a second billow zone that facilitates the flexible sheet, at the second billow zone, billowing away from the frame in a second direction responsively to the fluid pressure, the second direction being opposite to the first direction.

In some implementations, the panel is coupled to the frame by being secured at a plurality of points on the frame. In some implementations, the panel is coupled to the frame by being secured at a plurality of peripheral points, but not at a mid-region, of the panel. In some implementations, at least one lateral edge of the panel is unattached to the frame.

In some implementations, the augmenter is configured such that the billowing of the panel enhances coaptation between the second surface and the second leaflet during systole.

In some implementations, the anchor is fixedly coupled to the frame.

In some implementations, the anchor is pivotably coupled to the frame.

In some implementations, the anchor is a pair of clips.

In some implementations, the first leaflet has a root, the root being attached to an annulus of the valve. In some implementations, the anchor is configured to be anchored to the first leaflet closer to the lip than to the root.

In some implementations, the flexible sheet includes a film.

In some implementations, the augmenter is configured to billow during systole and to collapse during diastole, in an oscillating manner with each cardiac cycle.

In some implementations, the frame includes a shape-memory alloy.

In some implementations, at one end of the augmenter, the augmenter has a root portion, and at an opposite end of the augmenter, the augmenter has a lip portion. In some implementations, the anchor is configured to anchor the augmenter to the lip of the first leaflet such that the lip portion is downstream of the root portion.

In some implementations, at the lip portion, the panel is attached to the frame along an edge of the panel.

In some implementations, at the root portion, the anchor is coupled to the frame.

In some implementations, at a central portion of the augmenter, the anchor is coupled to the frame, the central portion being partway between the root portion and the lip portion.

In some implementations, the anchor is a clip. In some implementations, the frame and the clip are formed from a single piece of stock material.

In some implementations, the flexible sheet includes a fabric. The fabric can be configured in a variety of ways. In some implementations, the fabric includes a woven polymer. In some implementations, the fabric includes a knitted polymer.

In some implementations, the panel is a first panel extending over a first side of the frame, and the augmenter further includes a second panel, the second panel extending over an opposite side of the frame, the second panel having a central opening therethrough.

In some implementations, the central opening provides fluid communication with the first surface such that the first panel experiences the fluid pressure via the central opening, and responsively billows. In some implementations, the central opening is substantially central with respect to the second panel. In some implementations, the first panel and the second panel are attached to each other along at least one edge in a manner that defines a cavity.

In some implementations, at one end of the augmenter, the augmenter has a root portion, and at an opposite end of the augmenter, the augmenter has a lip portion. In some implementations, the anchor is configured to anchor the augmenter to the lip of the first leaflet such that the lip portion is downstream of the root portion, and/or at the lip portion, between the first panel and the second panel the augmenter defines a lip opening that provides passage for blood during diastole.

In some implementations, the central opening is shaped as a rounded square. In some implementations, a net area of the second panel, which is a gross area of the second panel minus an area of the central opening, is smaller than the area of the central opening.

In some implementations, the augmenter further includes a ventricular tongue extending away from the panel. In some implementations, the anchor is configured to anchor the augmenter to the lip of the first leaflet such that, at least during systole, the ventricular tongue becomes disposed on a ventricular side of the second leaflet.

In some implementations, a size of the ventricular tongue is adjustable.

In some implementations, the anchor is pivotably coupled to a central region of the frame, the pivotable coupling enabling the augmenter to pivot with respect to the first leaflet.

In some implementations, the augmenter is configured to pivot with respect to the first leaflet responsively to fluid pressure.

In accordance with some implementations, a system and/or an apparatus (e.g., useable with a valve of a heart, the valve having a first leaflet and a second leaflet, which first leaflet can have a lip) includes an augmenter (which can be the same as or similar to other augmenters herein) that includes a frame, and a flexible panel attached to the frame. In some implementations, an anchor coupled to the frame at a coupling point and configured to anchor the frame to the first leaflet such that, during ventricular systole, the flexible panel billows away from the frame in response to ventricular blood pressure. The anchor can be the same as or similar to any of the anchors described here or elsewhere herein.

In accordance with some implementations, a system and/or an apparatus includes an augmenter (which can be the same as or similar to other augmenters herein) for a first leaflet of a valve of a heart. In some implementations, the first leaflet has a lip, the valve has a second leaflet and an annulus, and the valve is disposed between an atrium and a ventricle of the heart. A root of the first leaflet can be attached to the annulus. In some implementations, the augmenter includes a first panel, and a second panel, articulatably coupled to the first panel by an articulatable coupling.

In some implementations, the augmenter is expandable by increasing an angular disposition between the first panel and the second panel, thereby increasing a dimension of an opening defined between the first panel and the second panel opposite the articulatable coupling. In some implementations, the augmenter is collapsible by reducing the angular disposition.

In some implementations, the augmenter includes an anchor, coupled to the first panel, and configured to anchor the first panel to the first leaflet. The anchor can be the same as or similar to any of the anchors described here or elsewhere herein.

In some implementations, the first panel is disposed against the first leaflet, and the articulatable coupling is disposed closer, than the lip and the opening, to the root.

In some implementations, the augmenter is sterile.

In some implementations, the anchor is configured to anchor the first panel to the first leaflet such that the augmenter expands during ventricular systole and collapses during ventricular diastole. In some implementations, the anchor is configured to anchor the first panel to the first leaflet such that the opening faces the ventricle.

In some implementations, the system further includes a flexible connector connected to the first panel and to the second panel in a manner that limits expansion of the augmenter by limiting the angular disposition between the first panel and the second panel.

In some implementations, the anchor is coupled to the first panel closer to the articulatable coupling than to a lip of the first panel.

In some implementations, each of the first panel and the second panel is substantially circular or elliptical in shape.

In some implementations, each of the first panel and the second panel is substantially rectangular in shape.

In some implementations, the first panel is sufficiently flexible to conform to a shape of the first leaflet.

In some implementations, the first panel is dimensioned such that the anchoring, by the anchor, of the first panel to the first leaflet is such that the first leaflet overhangs beyond the lip of the first leaflet.

In some implementations, the second panel is articulatably coupled to the first panel along an entire length of an edge of each panel.

In some implementations, the second panel is articulatably coupled to the first panel along a partial length of an edge of each panel.

In some implementations, the articulatable coupling includes stitches.

In some implementations, the articulatable coupling includes a hinge.

In some implementations, the articulatable coupling includes a flexure.

In some implementations, the anchor includes a clip.

In some implementations, the anchor includes a tissue-piercing element.

In some implementations, the augmenter is configured such that anchoring of the first panel to the first leaflet by the anchor increases an efficiency of the valve in inhibiting regurgitation of blood through the valve during ventricular systole.

In some implementations, the first panel is dimensioned such that, upon anchoring of the first panel to the first leaflet by the anchor, the first panel overhangs beyond the lip of the first leaflet.

In some implementations, the second panel is dimensioned to overhang beyond an edge of the first panel.

In some implementations, the first leaflet is a flailing leaflet, and the augmenter is configured to be anchored to the flailing first leaflet such that the second panel coapts with the second leaflet, thereby inhibiting flailing of the first leaflet.

In some implementations, the second leaflet is a flailing leaflet, and the augmenter is configured to be anchored to the first leaflet such that the second panel coapts with the flailing second leaflet, thereby inhibiting flailing of the second leaflet.

In some implementations, the augmenter is configured such that, while the first panel is anchored to the first leaflet by the anchor, expansion of the augmenter reduces regurgitation of blood through the valve.

In some implementations, the augmenter includes a polymer that is shaped to define the first panel and the second panel.

In some implementations, the augmenter includes a silicone that is shaped to define the first panel and the second panel.

In some implementations, the system further includes a tether coupled to and

extending from the first panel and configured to be attached to ventricular tissue while the anchor anchors the first panel to the first leaflet.

In some implementations, the anchor is a leaflet anchor, and the system/apparatus further includes a ventricular anchor, coupled to the tether, and configured to anchor the tether to the ventricular tissue while the leaflet anchor anchors the first panel to the first leaflet.

In some implementations, the ventricular tissue is tissue of a wall of the ventricle.

In some implementations, the ventricular tissue is tissue of a papillary muscle.

In some implementations, the ventricular anchor is coupled to the tether in a manner that facilitates adjustment of tension on the tether.

In some implementations, the system further includes a winch coupled to the ventricular anchor, the anchor being coupled to the tether via the winch such that the tension on the tether is adjustable by actuation of the winch.

In some implementations, at least the first panel includes a cell-growth-inducing material.

In some implementations, the second panel includes a cell-growth-inducing material.

In some implementations, the second panel includes and/or is formed from a cell growth-inhibiting material.

In some implementations, each of the first panel and the second panel include a flexible fabric.

In some implementations, the augmenter further includes at least one frame.

In some implementations, the at least one frame serves to reinforce the fabric.

In some implementations, the fabric of each of the first panel and the second panel is disposed over the at least one frame such that the at least one frame supports the fabric.

In some implementations, the at least one frame is shaped to define a hoop.

In some implementations, the at least one frame has an H shape defining a crossbar, the crossbar of the H traversing a face of the panel.

In some implementations, the at least one frame is positioned along at least one edge of each panel.

In some implementations, the at least one frame includes a first frame and a second frame, the first frame being articulatably coupled to the second frame.

In some implementations, the at least one frame includes a single integrated frame, having an articulation site at which the second panel is articulatably coupled to the first panel.

In some implementations, the single integrated frame includes an elastic material that biases the first panel and the second panel toward assuming a predetermined angular disposition therebetween.

In some implementations, the integrated frame includes an elastic material that defines a flexure connection between the first panel and the second panel.

In some implementations, the at least one frame is stitched to the fabric.

In some implementations, for each of the first panel and the second panel, the flexible fabric defines a first fabric layer, disposed over a first side of the at least one frame, the panel further includes a second fabric layer, disposed over a second side of the at least one frame, such that the frame is sandwiched between the first fabric layer and the second fabric layer.

In some implementations, the at least one frame and the fabric are dimensioned with respect to each other such that the at least one frame and the fabric are pretensioned.

In some implementations, when the fabric is disposed over the at least one frame, the at least one frame provides a specific form and shape to the panel.

In some implementations, the augmenter is biased toward the first panel and the second panel having a predetermined angular disposition therebetween.

In some implementations, the augmenter is biased to collapse by reducing the angular disposition between the second panel and the first panel.

In some implementations, the augmenter is biased to expand by increasing the angular disposition between the second panel and the first panel.

In some implementations, the first panel has a lip opposite the articulatable coupling, and/or a lateral edge extending between the articulatable coupling and the lip of the first panel. In some implementations, the second panel has a lip opposite the articulatable coupling, a lateral edge extending between the articulatable coupling and the lip of the second panel, and/or the opening is defined by the lip of the first panel and the lip of the second panel.

In some implementations, the system further includes a flexible connector connected to the first panel and to the second panel in a manner that limits expansion of the augmenter by limiting the angular disposition between the first panel and the second panel.

In some implementations, the flexible connector is located opposite the articulatable coupling, extending between the first panel and the second panel.

In some implementations, the flexible connector connects the lip of the first panel and the lip of the second panel.

In some implementations, the flexible connector connects a substantially central part of the lip of the first panel to a substantially central part of the lip of the second panel.

In some implementations, the flexible connector is a first flexible connector, and/or the augmenter further includes a second flexible connector. In some implementations, the first flexible connector connects a first lateral part of the lip of the first panel to a first lateral part of the lip of the second panel. In some implementations, the second flexible connector connects a second lateral part of the lip of the first panel, substantially opposite the first lateral part of the lip of the first panel, to a second lateral part of the lip of the second panel, substantially opposite the first lateral part of the lip of the second panel.

In some implementations, the flexible connector connects (i) a site on the first panel partway between the articulatable coupling and the lip of the first panel, and (ii) a site on the second panel partway between the articulatable coupling and the lip of the second panel.

In some implementations, the flexible connector connects the lateral edge of the first panel to the lateral edge of the second panel. In some implementations, the flexible connector includes a flexible third panel extending between the first panel and the second panel.

In some implementations, the flexible connector includes a cell-growth-inhibiting substance. In some implementations, the flexible connector includes a cell-growth-promoting substance. In some implementations, the flexible third panel connects the lip of the first panel to the lip of the second panel.

In some implementations, the third panel includes a fabric. In some implementations, the third panel includes a net.

In some implementations, the anchor is configured to anchor the first panel to the first leaflet such that the third panel coapts with the second leaflet during systole.

In some implementations, the anchor is configured to anchor the first panel to the first leaflet such that the third panel faces the ventricle during systole.

In some implementations, the lateral edge of the first panel is a first lateral edge of the first panel, the lateral edge of the second panel is a first lateral edge of the second panel, substantially aligned with the first lateral edge of the first panel, the first panel has a second lateral edge, substantially opposite the first lateral edge of the first panel, and extending between the articulatable coupling and the lip of the first panel. In some implementations, the second panel has a second lateral edge substantially opposite the first lateral edge of the second panel, extending between the articulatable coupling and the lip of the second panel, and substantially aligned with the second lateral edge of the first panel, and the third panel extends between the first lateral edge of the first panel and the first lateral edge of the second panel. In some implementations, the augmenter further includes a flexible fourth panel, extending between the second lateral edge of the first panel and the second lateral edge of the second panel.

In some implementations, the third panel and the fourth panel include folds configured to collapse inward when the opening collapses.

In some implementations, the flexible connector includes at least one connector element selected from: a wire, and a thread. In some implementations, the flexible connector includes exactly one connector element.

In some implementations, the anchor is coupled to the first panel closer to the articulatable coupling than to the lip of the first panel.

In some implementations, the anchor includes a toggle anchor. In some implementations, the toggle anchor has a blunt end and a sharp end and is configured to be advanced through the first leaflet sharp-end-first. In some implementations, the anchor is coupled to the first panel by a flexible thread.

In some implementations, the anchor is a first anchor, and the augmenter further includes a second anchor that is coupled to the first panel, and that is configured to anchor the first panel to the first leaflet.

In some implementations, the first anchor includes a first toggle anchor, and the second anchor includes a second toggle anchor.

In some implementations, the augmenter is configured such that, while (i) the first panel is anchored to the first leaflet, and (ii) the articulatable coupling is disposed closer to the root, than the lip and the opening, blood flow in the heart transitions the augmenter toward its expanded configuration during ventricular systole, and toward its collapsed configuration during ventricular diastole.

In some implementations, the system further includes a flexible connector connected to the first panel and to the second panel in a manner that limits expansion of the augmenter during ventricular systole.

In accordance with some implementations, a system and/or an apparatus includes an augmenter (which can be the same as or similar to other augmenters herein) useable and/or for use with a first leaflet of a valve of a heart of a subject (e.g., a living subject, a simulation, etc.). The first leaflet can have a lip, the valve being disposed between an atrium and a ventricle of the heart and having a second leaflet. In some implementations, the system/apparatus includes: an anchor, a first panel, a second panel, and/or a third panel.

In some implementations, each of the panels has a respective outer edge, and extends inwardly from the outer edge, such that the first, second, and third panels converge at an axis of the augmenter. In some implementations, the augmenter defines a panel plane that is substantially parallel with the axis, and/or the anchor is configured to anchor the first panel to the first leaflet.

In some implementations, the augmenter is sterile.

In some implementations, the augmenter includes a polymer that is shaped to define the first panel, the second panel, and the third panel.

In some implementations, the augmenter includes a silicone that is shaped to define the first panel, the second panel, and the third panel.

In some implementations, the anchor is configured to attach the first panel to the first leaflet such that the second panel extends away from the first panel toward the second leaflet.

In some implementations, the anchor is configured to anchor the first panel to the first leaflet such that the third panel extends away from the first panel toward the ventricle.

In some implementations, the anchor is coupled to the first panel closer to the outer edge of the first panel than to the axis of the augmenter.

In some implementations, each of the first panel, the second panel, and the third panel is substantially circular or elliptical in shape.

In some implementations, each of the first panel, the second panel, and the third panel is substantially rectangular in shape.

In some implementations, the first panel is sufficiently flexible to conform to a shape of the first leaflet.

In some implementations, the first panel is dimensioned such that the anchoring, by the anchor, of the first panel to the first leaflet is such that the third panel overhangs beyond the lip of the first leaflet. In some implementations, the second panel and the third panel collectively define a face. In some implementations, the anchor is configured to anchor the first panel to the first leaflet such that the first panel is disposed against the first leaflet. In some implementations, the face is presented as a coaptation surface to the second leaflet.

In some implementations, the anchor is configured to anchor the first panel to the first leaflet such that the third panel is disposed in the ventricle.

In some implementations, the anchor is configured to anchor the first panel to the first leaflet such that, during ventricular systole, the second panel extends, within the atrium, away from the first panel and across an upstream surface of the second leaflet.

In some implementations, the anchor is configured to anchor the first panel to the first leaflet such that, during ventricular systole, the second panel is disposed against an upstream surface of the second leaflet.

In some implementations, the anchor is configured to anchor the first panel to the first leaflet such that, during ventricular systole, a lip of the second leaflet contacts the third panel.

In some implementations, the system further includes a tether coupled to and extending from the first panel and configured to tether the first panel to ventricular tissue of the heart.

In some implementations, the anchor is a leaflet anchor, and the system/apparatus further includes a ventricular anchor, coupled to the tether, and configured to anchor the tether to the ventricular tissue while the leaflet anchor anchors the first panel to the first leaflet.

In some implementations, the ventricular tissue is tissue of a wall of the ventricle.

In some implementations, the ventricular tissue is tissue of a papillary muscle.

In some implementations, the ventricular anchor is coupled to the tether in a manner that facilitates adjustment of tension on the tether.

In some implementations, the system further includes a winch coupled to the ventricular anchor, the anchor being coupled to the tether via the winch such that the tension on the tether is adjustable by actuation of the winch.

In some implementations, at least the first panel includes a cell-growth-inducing material.

In some implementations, the second panel includes a cell-growth-inducing material.

In some implementations, the third panel includes a cell growth-inducing material.

In some implementations, the second panel includes a cell-growth-inhibiting material.

In some implementations, the third panel includes a cell growth-inhibiting material.

In some implementations, each of the first panel, the second panel, and the third panel includes a flexible fabric.

In some implementations, the augmenter further includes a frame.

In some implementations, the frame serves to reinforce the fabric.

In some implementations, the fabric of each of the first panel, the second panel, and the third panel is disposed over the frame such that the frame supports the fabric.

In some implementations, for each of the first, second, and third panels, the frame extends along the outer edge of the panel, substantially parallel with the axis.

In some implementations, the frame includes a single integrated frame defining an outline of the first panel, the second panel, and the third panel.

In some implementations, the integrated frame includes an elastic material that biases the first panel, the second panel, and the third panel toward assuming a predetermined angular disposition with respect to each other.

In some implementations, the integrated frame includes an elastic material.

In some implementations, the frame is stitched to the fabric.

In some implementations, for each of the first panel, the second panel, and the third panel, the flexible fabric defines a first fabric layer, disposed over a first side of the frame, and the panel further includes a second fabric layer, disposed over a second side of the frame, such that the frame is sandwiched between the first fabric layer and the second fabric layer.

In some implementations, the frame and the fabric are sized with respect to each other such that the frame and the fabric are pretensioned. In some implementations, when the fabric is disposed over the frame, the frame provides a specific form and shape to the first panel, the second panel, and the third panel.

In accordance with some implementations, a method (e.g., a method useable and/or for use with a real or simulated valve of a real or simulated heart of a real or simulated subject, the valve having a first leaflet and a second leaflet, and being disposed between an atrium and a ventricle of the heart) includes transluminally advancing an augmenter (which can be the same as or similar to other augmenters herein) to the heart.

In some implementations, the augmenter includes a first panel, a second panel articulatably coupled to the first panel, and/or an anchor. In some implementations, the method includes anchoring the first panel to the first leaflet using the anchor, such that the first panel of the augmenter is disposed against the first leaflet, and/or the second panel, in response to blood flow in the heart deflects away from the first panel and toward the second leaflet during ventricular systole. In some implementations, the second panel deflects away from the second leaflet and toward the first panel during ventricular diastole.

In some implementations, the method further includes sterilizing the augmenter.

In some implementations, anchoring the first panel to the first leaflet includes anchoring the first panel to the first leaflet such that the second panel, in response to blood flow in the heart, coapts with the second leaflet during ventricular systole, thereby reducing regurgitation through the valve.

In some implementations, deflection of the second panel during ventricular systole leads to the second panel coapting with the second leaflet.

In some implementations, the method further includes tethering the first panel to ventricular tissue using a tether connected to the first panel. In some implementations, the method further includes adjusting tension on the tether.

In some implementations, tethering the first panel to the ventricular tissue includes anchoring the tether to the ventricular tissue using a tissue anchor attached to the tether.

In some implementations, the method further includes coupling the tissue anchor to the tether via a winch. In some implementations, the method further includes adjusting tension on the tether by actuating the winch.

In some implementations, the method further includes attaching a flexible third panel between the first panel and the second panel.

In some implementations, deflection of the second panel during ventricular systole is accompanied by the flexible third panel coapting with the second leaflet.

In accordance with some implementations, a method (e.g., useable and/or for use with a real or simulated valve of a real or simulated heart of a real or simulated subject, the valve having a first leaflet and a second leaflet, and being disposed between an atrium and a ventricle of the heart) includes: transluminally advancing an augmenter (which can be the same as or similar to other augmenters herein) to the heart.

In some implementations, the augmenter includes: a first panel, a second panel, a third panel, and/or an anchor; anchoring the first panel to the first leaflet, wherein each of the panels has a respective outer edge, extends inwardly from the outer edge, such that the first, second, and third panels converge at an axis of the augmenter.

In some implementations, the augmenter defines a respective panel plane that is substantially parallel with the axis.

In some implementations, the method further includes sterilizing the augmenter.

In some implementations, anchoring the first panel to the first leaflet includes anchoring the first panel to the first leaflet while the second panel and the third panel are disposed within a delivery tool. In some implementations, the method further includes subsequently deploying the second panel and the third panel from the delivery tool.

In some implementations, deploying the second panel and the third panel from the delivery tool includes deploying the second panel and the third panel from the delivery tool such that the second panel and the third panel deflect away from each other.

In accordance with some implementations, a system and/or an apparatus includes a leaflet augmenter useable and/or for use at a valve of a heart of a subject (e.g., a living subject, a simulation, etc.), the valve having at least a first leaflet and a second leaflet. In some implementations, the system/apparatus and/or augmenter includes a first panel, and a second panel articulatably connected to the first panel.

In some implementations, a tissue-piercing element is coupled to the first panel and configured to anchor the first panel to the first leaflet such that, during ventricular systole, closure of the valve is accompanied by articulation of the second panel away from the first panel and toward the second leaflet.

In accordance with some implementations, a method for manufacturing an augmenter, the augmenter being usable and/or for use with a real or simulated valve of a real or simulated heart, the valve having a first leaflet and a second leaflet, includes cutting, from a shape memory alloy, a unitary skeleton that includes a frame-region and an anchor-region. In some implementations, the method includes in a first heat-setting procedure, heat-setting the anchor-region to define an anchor; and/or in a second heat-setting procedure, heat-setting the frame-region to define a frame.

In some implementations, the method includes forming a panel by attaching a sheet to the frame, the anchor being configured to anchor the augmenter to the first leaflet such that the panel coapts with the second leaflet during systole. The anchor can be the same as or similar to any of the anchors described here or elsewhere herein.

In some implementations, the method further includes sterilizing the augmenter.

In some implementations, heat-setting the anchor-region includes heat-setting the anchor-region using a first mold, and heat-setting the frame-region includes heat-setting the frame-region using a second mold.

In some implementations, the anchor includes a clip, and heat-setting the anchor-region to define the anchor includes heat-setting the anchor-region to define the clip.

In some implementations, the method includes performing the second heat-setting procedure subsequently to performing the first heat-setting procedure.

In some implementations, the method includes constraining the anchor-region in a heat-set shape during the second heat-setting procedure.

In some implementations, the cutting is performed by a laser.

In some implementations, the method further includes anchoring the augmenter to the valve by clamping the anchor to the first leaflet of the valve.

In some implementations, a first heat-setting mold is configured to mold the anchor-region, such that providing the frame and anchor with a first shape memory includes using the first heat-setting mold to mold the anchor-region of the shape memory alloy to a clamp shape.

In some implementations, a second heat-setting mold is configured to provide the frame with a curvature, such that that providing the frame with a second shape memory includes using the second heat-setting mold to provide the frame with a curvature that approximates that of the second leaflet.

In accordance with some implementations, a system and/or an apparatus includes an augmenter useable and/or for use with a valve of a heart, the valve having a first leaflet and a second leaflet. In some implementations, the augmenter includes a unitary skeleton, cut from a shape-memory alloy to define a frame-region and an anchor-region. In some implementations, the frame-region has been heat-set to define a frame, and/or the anchor-region has been heat-set to define an anchor.

In some implementations, the augmenter includes a sheet, attached to the frame in a manner that defines a panel. In some implementations, the anchor is configured to anchor the augmenter to the first leaflet such that the panel coapts with the second leaflet during systole.

In accordance with some implementations, a method (e.g., a method useable and/or for use with a real or simulated valve of a real or simulated heart of a real or simulated subject, the valve having a first leaflet and a second leaflet, and being disposed between an atrium and a ventricle of the heart) includes transluminally advancing an augmenter to the heart. In some implementations, the method includes a flexible pouch, having a downstream opening and an external surface, the pouch coupled to an anchor.

In some implementations, the method includes anchoring the anchor to the first leaflet, such that the pouch is anchored to the lip of the first leaflet by the anchor being anchored to the first leaflet, and responsively to ventricular systole, inflates via the downstream opening such that the external surface coapts against the opposing leaflet.

In some implementations, the method further includes using an imaging modality to view coaptation of the external surface against the opposing leaflet.

In some implementations, the method further includes sterilizing the augmenter.

In some implementations, the augmenter further includes an adjustable strap coupled to the pouch, and the method further includes adjusting a length of the strap to adjust an internal dimension of the pouch.

In some implementations, adjusting the length of the strap to adjust the internal dimension of the pouch includes shortening the strap to decrease the internal dimension of the pouch. In some implementations, adjusting the length of the strap to adjust the internal dimension of the pouch includes lengthening the strap to increase the internal dimension of the pouch. In some implementations, adjusting the length of the strap includes adjusting the length of the strap while viewing the pouch in the heart using an imaging modality.

In some implementations, the adjustable strap is a first adjustable strap, and adjusting the length of the adjustable strap includes adjusting the length of the first adjustable strap. In some implementations, the augmenter further includes a second adjustable strap coupled to the pouch, and/or the method further includes adjusting a length of the second adjustable strap to adjust the internal dimension of the pouch.

In some implementations, the opposing leaflet is a first opposing leaflet, the valve is a tricuspid valve having a second opposing leaflet, and/or anchoring the anchor to the first leaflet includes anchoring the anchor to the first leaflet such that, responsively to ventricular systole, the pouch inflates via the downstream opening, such that the external surface coapts against both the first opposing leaflet and the second opposing leaflet.

In some implementations, anchoring the anchor to the first leaflet includes anchoring the anchor to the first leaflet such that, responsively to ventricular systole, at least part of the pouch interposes between the first opposing leaflet and the second opposing leaflet.

In some implementations, the augmenter further includes an adjustable strap coupled to the pouch, and the method further includes adjusting a length of the adjustable strap to adjust an internal dimension of the pouch. In some implementations, adjusting the length of the adjustable strap to adjust the internal dimension of the pouch includes shortening the adjustable strap to decrease the internal dimension of the pouch. In some implementations, adjusting the length of the adjustable strap to adjust the internal dimension of the pouch includes lengthening the adjustable strap to increase the internal dimension of the pouch. In some implementations, adjusting the length of the strap includes adjusting the length of the adjustable strap while viewing the pouch in the heart using an imaging modality.

In some implementations, the adjustable strap having a length is a first adjustable strap having a first length, and/or adjusting the length of the adjustable strap includes adjusting the first length. In some implementations, the augmenter further includes a second adjustable strap coupled to the pouch and having a second length, and/or the method further includes adjusting the second length to adjust the internal dimension of the pouch.

In some implementations, adjusting the first length and the second length is performed in a manner that, responsively to ventricular systole, at least part of the pouch interposes between the first opposing leaflet and the second opposing leaflet.

In some implementations, the method further includes using an imaging modality to view coaptation of the external surface against the first opposing leaflet and the second opposing leaflet.

In some implementations, using the imaging modality to view coaptation of the external surface against the first opposing leaflet and the second opposing leaflet is performed while adjusting the first adjustable strap and the second adjustable strap.

Any of the above method(s) and any methods of using the systems, assemblies, apparatuses, devices, etc. herein can be performed on a living subject (e.g., human, or other animal) or on a simulation (e.g., a cadaver, cadaver heart, imaginary person, simulator, etc.). With a simulation, the body parts can optionally be referred to as “simulated” (e.g., simulated heart, simulated tissue, etc.) and can optionally comprise computerized and/or physical representations.

Any of the above systems, assemblies, devices, apparatuses, components, etc. can be sterilized (e.g., with heat, radiation, ethylene oxide, hydrogen peroxide, etc.) to ensure they are safe for use with patients, and the methods herein can comprise (or additional methods comprise or consist of) sterilization of one or more systems, devices, apparatuses, components, etc. herein (e.g., with heat, radiation, ethylene oxide, hydrogen peroxide, etc.).

The present disclosure will be more fully understood from the following detailed description of implementations thereof, taken together with the drawings, in which:

### BRIEF DESCRIPTION OF THE DRAWINGS

FIGS. 1A-C are schematic illustrations of a native heart valve of a subject;

FIGS. 2A-F, 3A-F, 4A-F, 5A-F, and 6A-C are schematic illustrations of example augmenters, in accordance with some implementations;

FIGS. 7-8 are schematic illustration of examples of systems comprising an augmenter implanted at a native valve of a heart, in accordance with some implementations;

FIGS. 9A-B are schematic illustrations showing an augmenter implanted at a native valve of a heart, in accordance with some implementations;

FIGS. 10A-C are schematic illustrations of an augmenter according to some implementations;

FIG. 11 is a schematic illustration showing an augmenter, e.g., from FIGS. 10A-B, implanted at a valve of a heart, in accordance with some implementations;

FIGS. 12A-C are schematic illustrations of an example augmenter, including aesthetic features thereof, according to some implementations;

FIG. 13 is schematic illustrations showing an example augmenter, e.g., from FIGS. 12A-C, implanted at a native valve of a heart, in accordance with some implementations;

FIGS. 14A-B and 15A-B are schematic illustrations of an example augmenter, including aesthetic features thereof, according to some implementations;

FIG. 16 is schematic illustration showing an example augmenter, e.g., from FIGS. 15A-B, implanted at a native valve of a heart, in accordance with some implementations;

FIG. 17 is a schematic illustration of an example augmenter, including aesthetic features thereof, according to some implementations;

FIG. 18 is schematic illustration showing an example augmenter, e.g., from FIG. 17, implanted at a native valve of a heart, in accordance with some implementations;

FIGS. 19A-D are schematic illustrations of an example augmenter, including aesthetic features thereof, according to some implementations;

FIGS. 20A-B are schematic illustrations showing an example augmenter, e.g., from FIGS. 19A-D, implanted at a native valve of a heart, in accordance with some implementations;

FIG. 21 is a schematic illustration of an example augmenter, including aesthetic features thereof, according to some implementations;

FIGS. 22, 23, 24, and 25 are schematic illustrations of an example augmenter, including aesthetic features thereof, according to some implementations;

FIGS. 26A-B are schematic illustrations showing an example augmenter, e.g., from FIGS. 22-25, implanted at a native valve of a heart, in accordance with some implementations;

FIGS. 27A-B, 28A-B, 29A-B, 30A-B, 31A-B, 32A-B, 33A-B, 34A-B, 35A-D, and 36 are schematic illustrations showing an example augmenter with a stabilizer, including aesthetic features thereof, implanted at a native valve of a heart, in accordance with some implementations;

FIGS. 37A-C and 38A-B are schematic illustrations of an example augmenter, including aesthetic features thereof, according to some implementations;

FIGS. 39A-B and 40A-B are schematic illustrations showing an example augmenter, including aesthetic features thereof, comprising a frame and implanted at a native valve of a heart, in accordance with some implementations;

FIGS. 41, 42A-D, and 43A-C are schematic illustrations showing an example augmenter, including aesthetic features thereof, comprising an expandable clasp element, and its implantation at a native valve of a heart, in accordance with some implementations;

FIGS. 44A-B are schematic illustrations showing an example augmenter having a cone shape and disposed between the leaflets of a valve of the heart;

FIGS. 45A-C are schematic illustrations showing an example augmenter having a cone shape and connected to an anchor via a rod, in accordance with some implementations;

FIGS. 46A-D, 47A-B, 48A-B, and 49A-C are schematic illustrations showing an example augmenter having a cone shape and connected to an anchor via a U-shaped handle, in accordance with some implementations;

FIGS. 50 and 51 are schematic illustrations showing example stabilizers for an augmenter;

FIGS. 52 and 53A-B are schematic illustrations showing an example augmenter having a cone shape with an apical vent, and/or with downstream circumferential support legs, in accordance with some implementations; and

FIGS. 54A-C and 55A-C are schematic illustrations showing an example augmenter having a teepee-shaped frame, in accordance with some implementations.

### DETAILED DESCRIPTION

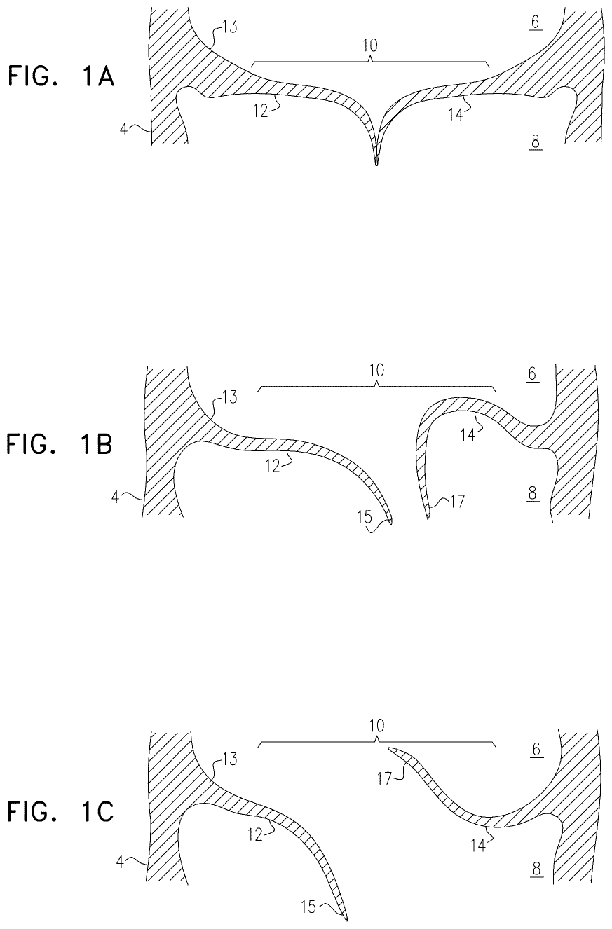

Reference is now made to FIGS. 1A-1C, which are schematic illustrations of part of a native valve 10 of a heart 4 of a subject, in various conditions. FIG. 1A represents a heart having normal and/or healthy anatomy and function. Valve 10 includes a first leaflet 12 and at least a second leaflet 14, each of which has a root 13 at which it is attached, e.g., to an annulus of the valve. For simplicity, only root 13 of first leaflet 12 has been labeled. Valve 10 can be located between an atrium 6 and a ventricle 8 of the heart—e.g., can be an atrioventricular valve such as a mitral valve or a tricuspid valve. In some implementations, valve 10 can be a valve between a chamber of a heart and a major blood vessel leading into or out of the chamber. FIG. 1A shows heart 4 in ventricular systole, during which leaflets 12 and 14 coapt, thereby preventing backflow of blood from ventricle 8 into atrium 6.

FIGS. 1B and 1C show heart 4 having a valve 10 in which leaflets 12 and 14 fail to close completely during ventricular systole—e.g., the leaflets fail to completely coapt with each other. The cause of inadequate closure may be valve prolapse or other damage or structural change to the leaflets, roots, chorda tendinea, or annulus of the valve. As a result, regurgitation through valve 10 occurs during ventricular systole. FIG. 1B shows leaflet 14 not reaching leaflet 12, whereas FIG. 1C shows lip 17 of leaflet 14 flailing upward into atrium 6. Failure of leaflets 12 and 14 to coapt renders valve 10 ineffective at preventing regurgitation of blood from ventricle 8 to atrium 6 during ventricular systole.

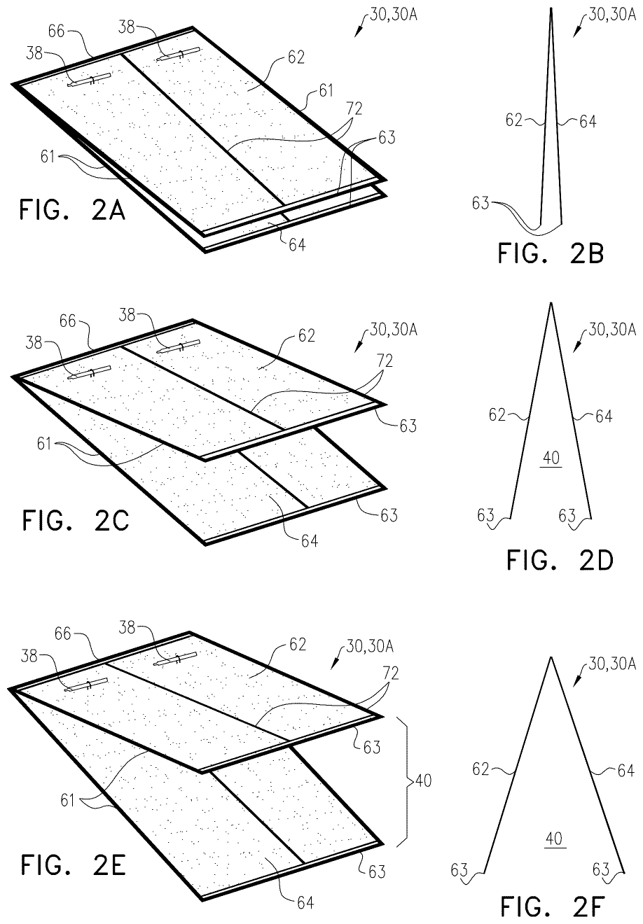

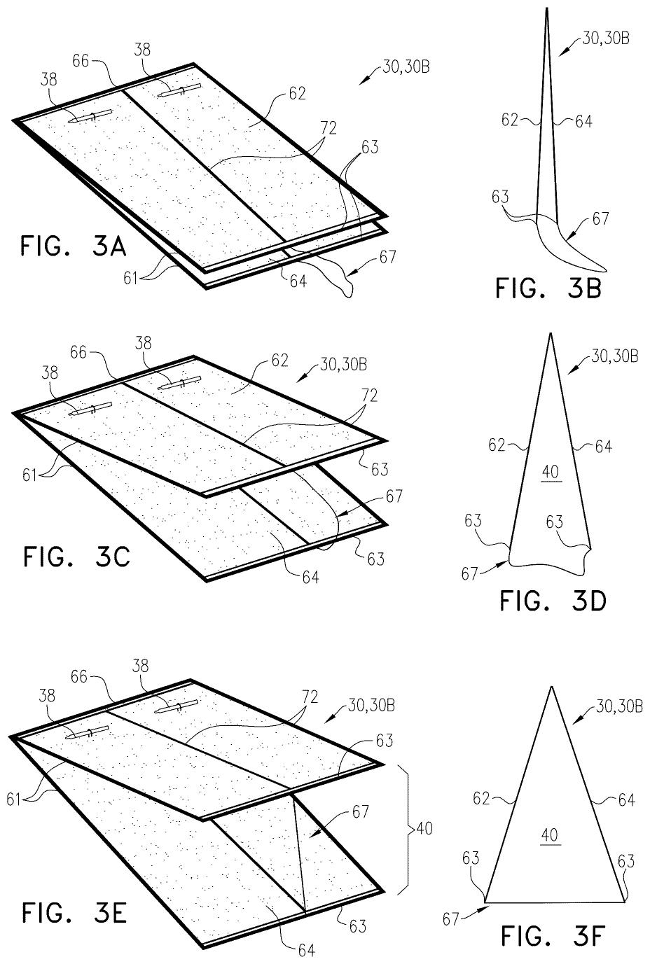

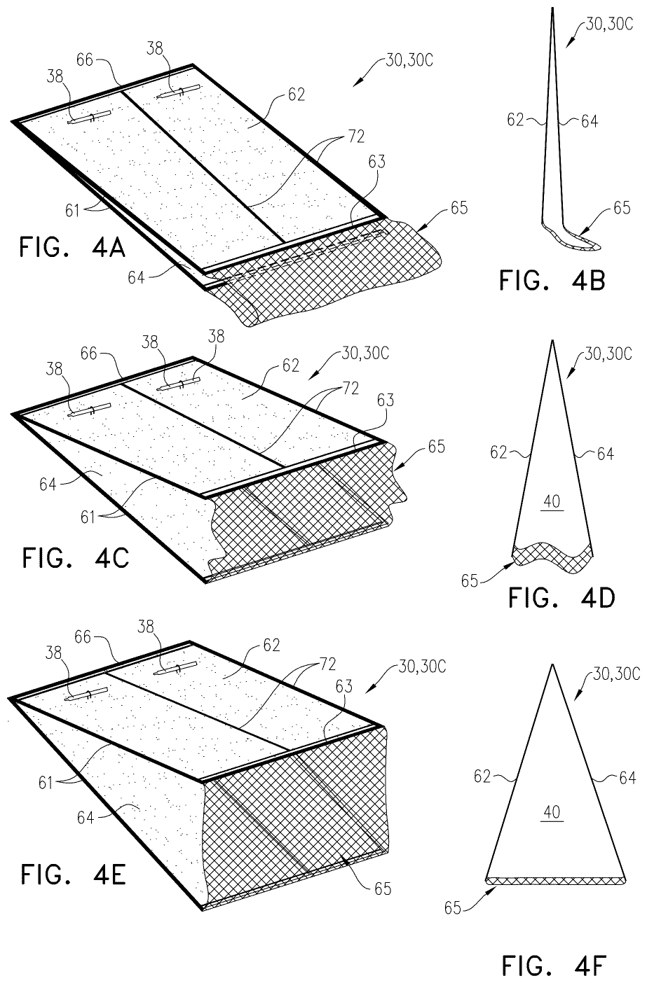

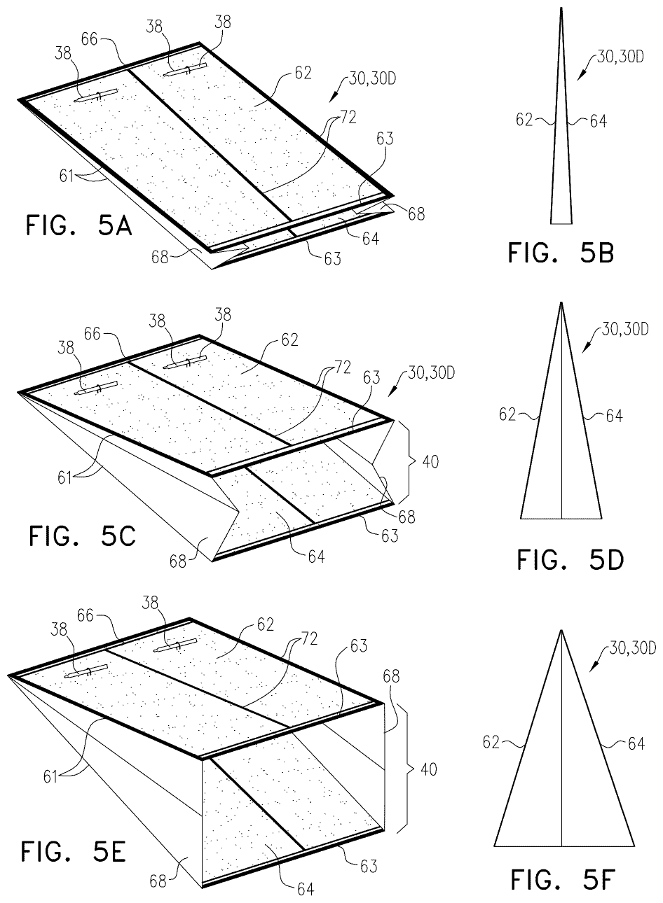

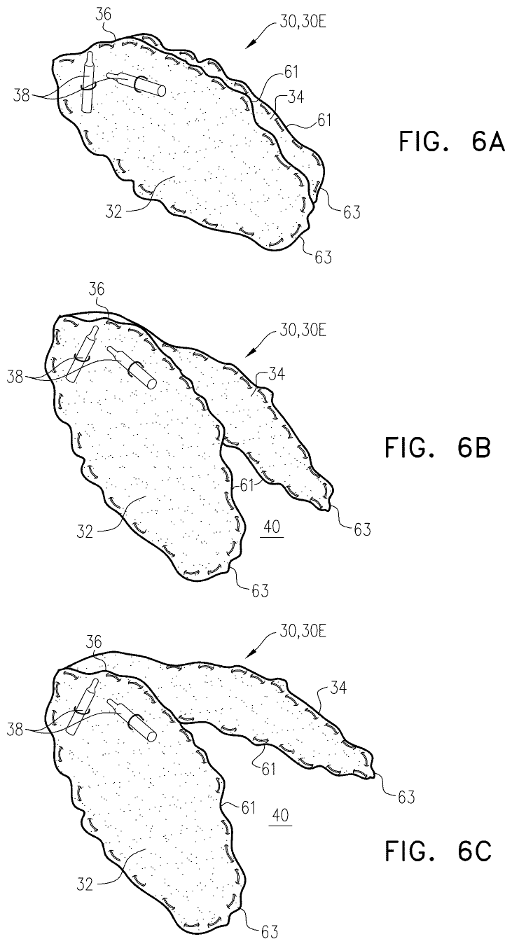

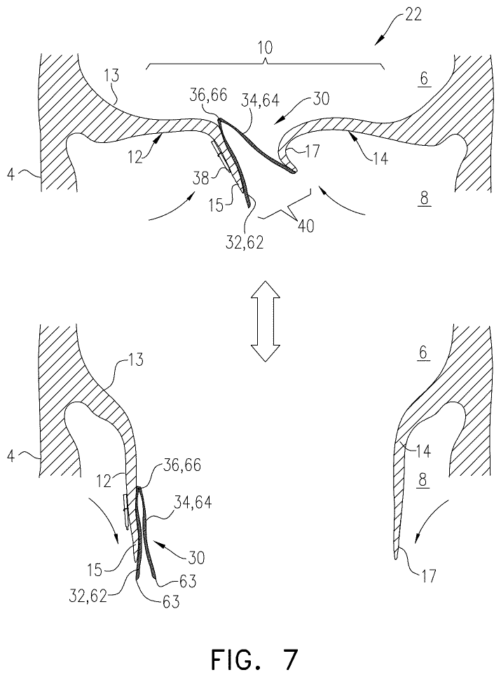

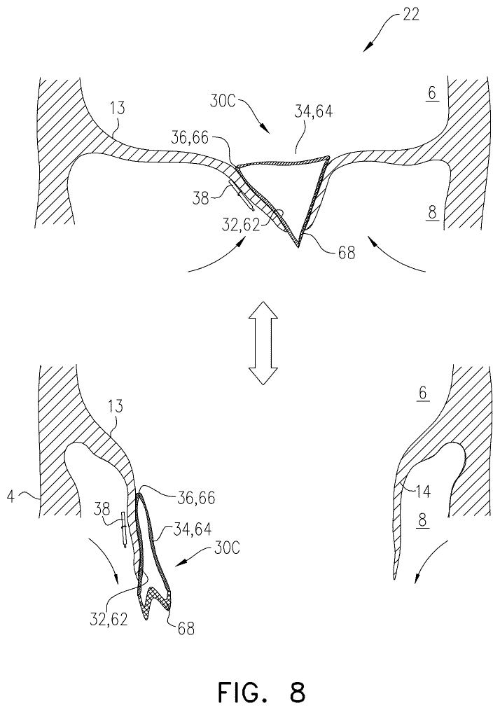

Reference is made to FIGS. 2A-F, 3A-F, 4A-F, 5A-F, and 6A-C, which are schematic illustrations of various variants of an augmenter 30 (e.g., a leaflet augmenter), in accordance with some implementations. FIGS. 2A-F show an augmenter 30A, FIGS. 3A-F show an augmenter 30B, FIGS. 4A-F show an augmenter 30C, FIGS. 5A-F show an augmenter 30D, and FIGS. 6A-C show an augmenter 30E, in accordance with some implementations. Example variants of augmenter 30, e.g., as implanted into the heart, are subsequently shown in FIGS. 7, 8, and 9A-B. It is to be understood that the augmenter 30 in FIGS. 7, 8, and/or 9-B can represent any one of the variants (30, 30A, 30B, 30C, 30D, or 30E) shown in FIGS. 2A-6C.

Each of augmenters 30A, 30B, 30C, and 30D comprises panels 62 and 64, and augmenter 30E comprises panels 32 and 34. In each variant, panels 62 and 64, or panels 32 and 34, are coupled at an articulatable coupling 66 along one adjacent/parallel edge. In the examples shown, each of panels 62 and 64 has a substantially rectangular shape, and each of panels 32 and 34 has a substantially elliptical shape. In the following descriptions, reference to panels 62 and 64 can be understood to apply also to panels 32 and 34.

Each of panels 62 and 64 can comprise a flexible material. In some implementations, each panel can have a single layer of the flexible material. In some implementations, the panels comprise a double layer of a biocompatible fabric or mesh webbing. The dimensions of panels 62 and 64 can be provided according to the size of the valve and/or of a regurgitating part of the valve that the augmenter 30 is intended to repair.

The articulatable coupling 66 between panels 62 and 64 is such that augmenter 30 is expandable by increasing an angular disposition between the panels, and collapsible by reducing the angular disposition. Expansion of the augmenter increases a dimension of an opening 40 defined between the panels, opposite the articulatable coupling, and collapsing of the augmenter decreases the dimension of the opening. In some implementations, this behavior may be described as panel 64 billowing away from panel 62 (e.g., the frame of panel 62) responsively to fluid pressure during ventricular systole.

Each panel (e.g., the flexible material thereof) can be endowed with cell growth-modulating properties. In some implementations, the fabric can have tissue growth-promoting or tissue growth-inhibiting properties. In some implementations, one side of each panel can comprise fabric having different properties than the fabric on the opposite side of the panel, and/or the fabric of one panel can have different properties than the fabric of the other panel.

FIGS. 2A-B, 3A-B, 4A-B, 5A-B, and 6A show augmenter 30 (e.g., various examples thereof) in a state in which panels 62 and 64 have a minimal angular separation between them, e.g., a collapsed state. In the collapsed state, lips 63 can be adjacent to each other (and even in contact with each other) and augmenter 30 can appear substantially as a single panel, e.g., panels 62 and 64 can behave substantially as a single panel.

FIGS. 2E-F, 3E-F, 4E-F, 5E-F, and 6C show augmenter 30, e.g., various examples thereof, in a state in which panels 62 and 64 have a maximal angular separation between them, e.g., the largest angular separation allowed by the configuration of the augmenter. Expansion of the augmenter can be limited by articulatable coupling 66 or can be limited by a flexible connector (e.g., as described hereinbelow).

FIGS. 2C-D, 3C-D, 4C-D, 5C-D, and 6B show augmenter 30 (e.g., various examples thereof) in an intermediate state, e.g., via which the augmenter can transition between the collapsed and expanded states.

Coupling 66 between panels 62 and 64 can comprise, for example, a hinge, stitching, a flexure, metal formed in a specific configuration, e.g., nitinol having a specific shape and memory, or other flexible closure, e.g., another means of articulatably coupling the two panels along one edge. The articulatable coupling 66 can connect panels 62 and 64 along the entire length of an edge of each panel. In some cases, the panels are articulatably coupled along a partial length of an edge of each panel.

In some implementations, the articulation is biased such that panel 62 and panel 64 have a predetermined angular disposition therebetween, e.g., the coupling can be designed with a stiffness that tends to hold the two panels a predetermined distance apart under conditions of no pressure. The augmenter 30 can be biased to collapse by reducing the angular disposition between the second panel and the first panel. The augmenter 30 can be biased to expand by increasing the angular disposition between the second panel and the first panel. In some cases, the predetermined angular disposition can be greater than 10 degrees and/or less than 120 degrees, e.g., 10-120 degrees (e.g., 20-90 degrees, such as 20-60 degrees)—e.g., such that the augmenter is biased to expand or open.

Bias towards collapse may be desirable in cases in which, e.g., the valve is stenotic, or the ventricle is small e.g., so as to maintain the effective valve orifice as open as possible during diastole (e.g., such that the augmenter does not substantially inhibit blood flow during diastole). Bias toward expansion may be advantageous in cases in which it is particularly challenging to achieve coaptation during diastole, e.g., a large gap exists between the leaflets of the valve, and/or hemodynamics of the heart of the subject are not optimal for blood-flow-induced opening of the augmenter. Thus, the advantage of using an augmenter that is biased toward either collapse or expansion may depend on factors such as the anatomical features and pathology of the valve and heart under treatment.

In some implementations, the first and second panels of augmenter 30 can be substantially circular or elliptical in shape, e.g., as shown for panels 32 and 34 of augmenter 30E. In some implementations, the panels can be substantially rectangular in shape, e.g., as shown for panels 62 and 64 of augmenters 30A, 30B, 30C, and 30D. It is to be noted that the scope of the present disclosure also includes panels of any other suitable shape.

Panels of any shape can comprise a flexible fabric, or can comprise a plastic or flexible substance, e.g., a silicone or a polymer, which is shaped to define the first panel and the second panel. Panels 62 and 64 can be sufficiently flexible to conform to the shape of the leaflet, e.g., the two panels can be shaped to conform to the outline of a valve leaflet, or they can have a different shape. Panel 62 can be dimensioned to overhang beyond lip 15 of leaflet 12, e.g., as further described in FIGS. 7, 8, and 9A-B hereinbelow.

Panels 62 and 64 can be self-supporting, such that a flexible connector to limit opening 40 is not necessary. Panels 62 and 64 can be reinforced with a frame 72 (or a pair of frames), e.g., with metal inserts, strips of metal such as nitinol, or metal wire. The frame can be woven onto or into the fabric of the panels. In some implementations, a stiffening mechanism can be metal threads or fine strips woven into the fabric.

In some implementations, and as shown, frame 72 extends along one or more edges and/or lips of the panels. In some implementations, and as shown, frame 72 can span across a central area of each panel, between coupling 66 and lip 63, e.g., such that the frame defines an H shape. Each panel of augmenter 30 can comprise a biocompatible fabric in two layers, between or within which a wire loop is fastened, the wire loop providing flexible stiffness to the panel.

The flexible fabric can define a first fabric layer, disposed over a first side of frame 72. The panel can further comprise a second fabric layer, disposed over a second side of frame 72, such that the frame is sandwiched between the first fabric layer and the second fabric layer. The fabric layers on each side of each panel can comprise the same or different types of fabric. The different types of fabric can differ, e.g., in their tissue growth-promoting properties.

Augmenter 30 can comprise one or more anchors 38, configured to anchor panel 62 (and thereby the augmenter as a whole), to a first leaflet 12 of a valve, as demonstrated in FIGS. 7-9. For example, and as described in more detail hereinbelow, anchors 38 can be configured (e.g., positioned with respect to panel 62) to anchor panel 62 to leaflet 12 such that articulatable coupling 66 is disposed closer to root 13 than is either lip 63 or opening 40.

Anchors 38 can comprise, for example, pins, tissue-piercing elements, T-bars, threads, clips, sutures, or locking elements.

In some implementations, anchors 38 can be provided and/or introduced separately, and subsequently attached to panel 62 (e.g., during implantation of augmenter 30). In some implementations, augmenter 30 can be provided with anchors 38 pre-attached to panel 62.

The location at which anchor 38 is attached to panel 62 can be near coupling 66, e.g., nearer to the coupling than to lip 63, such as being substantially at coupling 66. Anchors 38 can be flexibly connected to panel 62 such that the anchors are movable and can be directed to penetrate a valve leaflet. In some cases, the anchors are toggle anchors having one end sharper than the other, e.g., a sharp end used to pierce leaflet 12, and a blunt end. The anchors can be attached to the augmenter, e.g., to panel 62, via a flexible thread, string, or wire woven into panel 62.

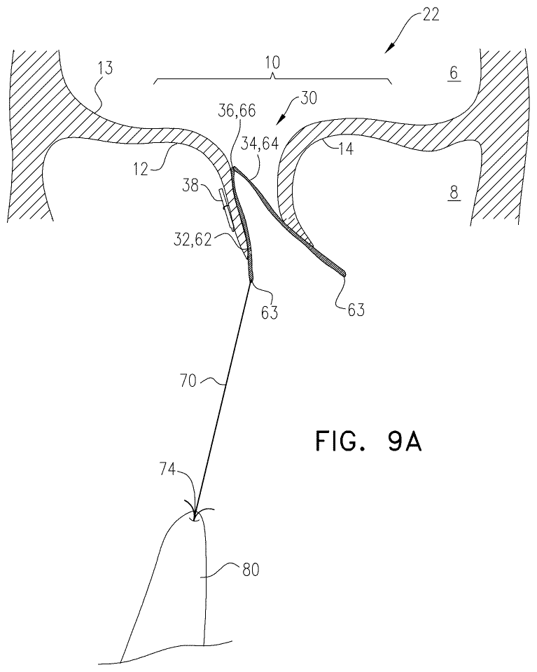

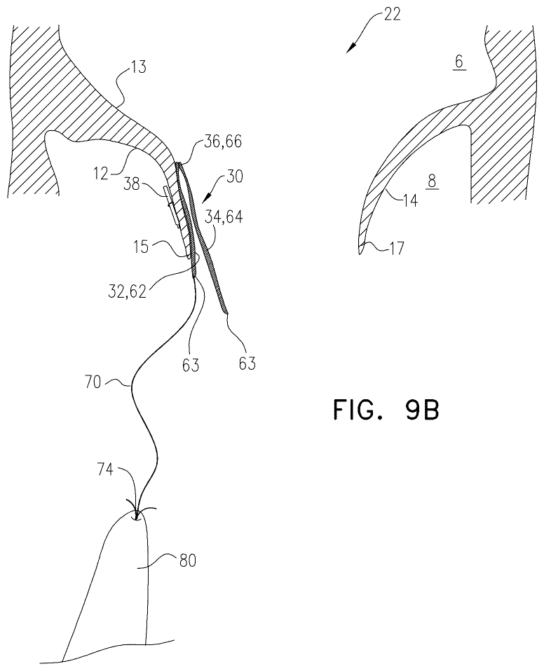

In some implementations, panels 62 and 64 are substantially the same size and/or shape. In some implementations, panel 64 is a different size (e.g., a different length) than panel 62. For example, panel 64 can be larger (e.g., longer) than panel 64, such that panel 64 extends beyond the lip 63 of panel 62, e.g., as described hereinbelow with reference to FIG. 9A. In some implementations, panel 62 can extend beyond lip 63 of panel 64.

FIGS. 2A to 2F show an augmenter 30A, which is a variant of augmenter 30, in accordance with some implementations. FIGS. 2A, 2C, and 2E show augmenter 30A in a sequence of conformations from closed (FIG. 2A) to partially open (FIG. 2C) to maximally open (FIG. 2E), as viewed isometrically. FIGS. 2B, 2D, and 2F show the same sequence of conformations from closed to maximally open, as viewed in two dimensions from one side of the augmenter. In general, FIGS. 2A and 2B can be considered to represent the conformation of augmenter 30A during diastole (after the augmenter has been implanted), and FIGS. 2E and 2F can be considered to represent its conformation during systole.

In FIGS. 2A-F, frame 72 serves to support and/or strengthen panels 62 and 64, e.g., providing shape and strength. Frame 72 can be a single integrated frame, defining articulatable coupling 66; in general, frame 72 is referred to herein in the singular. However, in some implementations, discrete frames can be provided for each panel of augmenter 30—and these discrete frames can be articulatably coupled to each other at coupling 66. It is to be noted that in implementations in which a discrete frame 72 is provided for each panel of augmenter 30A, the frames are coupled only at articulatable coupling 66.

In contrast to implementations 30B, 30C, and 30D shown in FIGS. 3A-F to 5A-F, respectively, augmenter 30A lacks a discrete flexible connector between panels 62 and 64 to limit the angular expansion. Rather, frame 72 itself can provide limiting functionality with regard to angular expansion of augmenter 30A. In FIGS. 2A-2F, frames 72 are coupled only at articulatable coupling 66.

FIGS. 3A to 3F show an augmenter 30B, which is a variant of augmenter 30, in accordance with some implementations. FIGS. 3A, 3C, and 3E show augmenter 30B in a sequence of conformations from closed (FIG. 3A) to partially open (FIG. 3C) to maximally open (FIG. 3E), as viewed isometrically. FIGS. 3B, 3D, and 3F show the same sequence of conformations from closed to maximally open, as viewed in two dimensions from one side of the augmenter. In general, FIGS. 3A and 3B can be considered to represent the conformation of augmenter 30B during diastole (after the augmenter has been implanted), and FIGS. 3E and 3F can be considered to represent its conformation during systole.

Augmenter 30B further comprises a flexible connector 67 such as a string, thread, or wire. Connector 67 is attached to, and between, lip 63 of panel 62 and lip 63 of panel 64, opposite the articulatable coupling 66. Connector 67 constrains the angular disposition between panels 62 and 64 to the extent of opening 40. Connector 67 can be sized as required according to a desired maximal angular disposition between panels 62 and 64, defining the extent of opening 40 when the device is in an open configuration.

During systole, connector 67 prevents panel 64 from flapping wider than the predetermined or desired extent of opening 40. During diastole, when panels 62 and 64 tend to collapse toward each other, connector 67 is not under tension and can hang from the lips of panels 62 and 64. In some implementations, more than one connector can be attached between panels 62 and 64, e.g., a connector on each corner framing opening 40, or several connectors along lips 63 of panels 62 and 64. Similarly, In some implementations one or more connectors 67 can connect lateral sides 61 of panels 62 and 64. Furthermore, In some implementations one or more connectors 67 can connect other regions of the panels, e.g., the middle of each panel (e.g., compared to the position of connector 67 in FIGS. 3A-F, closer to articulatable coupling 66). The number and material composition of the connectors may depend on the desired strength of the connector. It may also depend on the size and shape of the panels, and in some cases, on the type of articulation.

FIGS. 4A to 4F show an augmenter 30C, which is a variant of augmenter 30, in accordance with some implementations. As in FIGS. 2 and 3, FIGS. 4A, 4C, and 4E show augmenter 30C in a sequence of conformations from closed (FIGS. 4A, 4B) to partially open (FIGS. 4C, 4D) to maximally open (FIGS. 4E, 4F), as viewed isometrically. FIGS. 4B, 4D, and 4F show the same sequence of conformations from closed to maximally open, as viewed in two dimensions from one side of the augmenter. In general, FIGS. 4A and 4B can be considered to represent the conformation of augmenter 30B during diastole (after the augmenter has been implanted), and FIGS. 4E and 4F can be considered to represent its conformation during systole.

In FIGS. 4A-F is shown that the augmenter can further comprise a third panel 65 of a flexible material. As well as being flexible, panel 65 can be highly permeable—e.g., allows blood to flow therethrough. Third panel 65 can comprise a variety of flexible materials having openings or holes of a predetermined size, e.g., a mesh fabric or net of metal wire or interwoven threads. In some implementations, the holes are of such size as to allow free passage of blood therethrough. Panel 65 can comprise a tissue growth-inhibiting substance.

The third panel 65 is disposed along and between lips 63 of panel 62 and panel 64, connecting the edges of panel 62 and panel 64 opposite the articulation, typically spanning opening 40. Panel 65 attaches to lips 63 of panels 62 and 64 opposite the hinge, providing a means of limiting the flap of the augmenter to a predetermined angular opening. Therefore panel 65 can be considered to be a flexible connector, e.g., functioning similarly to flexible connector 67 described hereinabove, mutatis mutandis. Panel 65 can be sized as required according to a desired maximal angular disposition between panels 62 and 64, defining opening 40 when the device is in an open configuration.

Alternatively, or additionally, panel 65 can be designed to coapt with the second leaflet 14, providing support for the second leaflet and preventing it from flailing into the atrium during ventricular systole (e.g., as described with reference to FIG. 8).

FIGS. 5A to 5F show an augmenter 30D, which is a variant of augmenter 30, in accordance with some implementations. FIGS. 5A, 5C, and 5E show augmenter 30D in a sequence of conformations from closed (FIGS. 5A, 5B) to partially open (FIGS. 5C, 5D) to maximally open (FIGS. 5E, 5F), as viewed isometrically. FIGS. 5B, 5D, and 5F show the same sequence of conformations from closed to maximally open, as viewed in two dimensions from one side of the augmenter. In general, FIGS. 5A and 5B can be considered to represent the conformation of augmenter 30D during diastole (after the augmenter has been implanted), and FIGS. 5E and 5F can be considered to represent its conformation during systole.

Augmenter 30D comprises panels 62 and 64, and articulatable coupling 66. In some implementations, panels 62 and 64 are supported, or their angular separation is constrained, by one or more third panels 68, e.g., accordion folds, flexible fabric, net, or other flexible sheet, connecting the lateral sides of panels 62 and 64. Panels 68 can be disposed perpendicular to articulatable coupling 66. Panels or folds 68 provide stability to the augmenter and frame opening 40 opposite the articulation. When completely closed, as in FIGS. 5A-5B, panels 68 are fully collapsed or folded (e.g., between panels 62 and 64), such that panels 62 and 64 are nearly adjacent. When fully open, as in FIGS. 5E-5F, the extent of opening 40 can be limited by the size of the folds or additional panels 68 on either side. Therefore panels 68 can be considered to be flexible connectors, e.g., functioning similarly to flexible connector 67 described hereinabove, mutatis mutandis. Panels 68 can be sized as required according to a desired maximal angular disposition between panels 62 and 64, creating opening 40 when the device is in an open configuration. In some implementations, the folds comprise flexible fabric that can crumple or balloon outward upon collapse or closure of augmenter 30D.

FIGS. 6A to 6C show an augmenter 30E, which is a variant of augmenter 30, in accordance with some implementations. Augmenter 30E is shown in a sequence of conformations from closed (FIG. 6A) to partially open (FIG. 6B) to maximally open (FIG. 6C), as viewed isometrically. In general, FIG. 6A can be considered to represent the conformation of augmenter 30E during diastole (after the augmenter has been implanted), and FIG. 6C can be considered to represent its conformation during systole.

Augmenter 30E has a shape in which panels 32 and 34, corresponding to panels 62 and 64 in implementations 30A to 30D, have an elliptical (e.g., circular) outline. The panels are stitched together along one region, creating an articulation 36. As in the implementations shown in FIGS. 2A-F to 5A-F, opening 40 can be a free space or opening at the lips 63 of panels 32 and 34. Augmenter 30E can be self-supporting, or can be collapsed or restrained in dimension of angular opening by a string, wire, net, or lateral accordion folds, or any of the other mechanisms for the flexible connector mentioned in the descriptions of FIGS. 2 to 5. That is, descriptions of panels 62 and 64 apply to panels 32 and 34 as well. In implementations in which a flexible connector is included, the connector can be sized as required according to a desired maximal angular disposition between panels 32 and 34, creating opening 40 when the device is in an open configuration.

Panels 32 and 34 can comprise a flexible fabric. The fabric can be disposed over and supported by a frame (not shown) comprising metal, plastic, or other material with bendable stiffness, such that the frame supports the fabric. In some implementations, each panel can have its own frame, the individual frames articulatably coupled to each other. In some implementations, a single integrated frame that extends between both panels and has an articulation site, can be used. Each panel can comprise two layers of the fabric, sandwiching the frame there between. In some implementations, the frame can be held within the two layers of fabric without being directly stitched to the panel itself (e.g., within each panel, the frame is constrained within a pocket defined by the two layers of fabric). In some implementations, the frame can be stitched directly to the fabric.

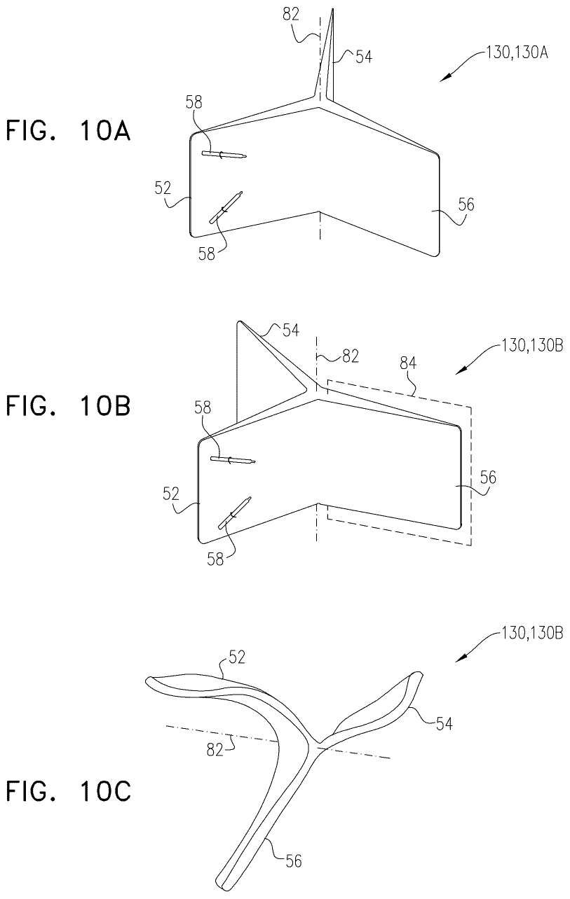

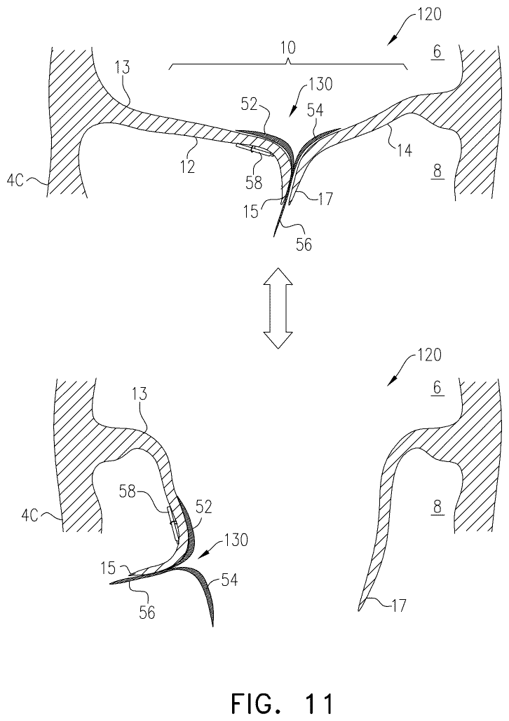

Reference is made to FIGS. 7, 8, and 9A-B, which are schematic illustrations of a system 22 in which augmenter 30 (e.g., a variant thereof) is implanted at valve 10, in accordance with some implementations. Implementations of the system can be used to treat cases of valve dysfunction, e.g., prolapse, regurgitation, or leaflet flail. Augmenter 30 can be anchored to leaflet 12, or in some implementations, to leaflet 14. In some implementations, augmenter 30 is anchored to a dysfunctional leaflet. In some implementations, augmenter 30 is anchored to a healthy leaflet (e.g., opposite a dysfunctional leaflet). In some implementations, a respective augmenter 30 is anchored to each of the leaflets. In each figure, the upper image represents valve 10 of heart 4 during systole, and the lower image represents the valve during diastole. These conventions apply also to FIG. 11, described in more detail below, for augmenter 130.

Although the following description refers to panels 62 and 64, the description is equally applicable to variants of augmenter 30 that comprise panels 32 and 34, mutatis mutandis.

FIG. 7 shows system 22 after augmenter 30 has been transluminally advanced to heart 4, and panel 62 has been anchored to leaflet 12 using anchor 38. Augmenter 30 can be any one of augmenter 30A, 30B, 30C, 30D, or 30E, from FIGS. 2A-F to 6A-C, or a combination of these variants.

Augmenter 30 is configured to be secured to leaflet 12, such as via an anchor 38, e.g., as shown in FIGS. 7, 8, and 9A-B. Augmenter 30 can be secured to either a healthy leaflet (e.g., as shown in FIG. 7), or to a dysfunctional leaflet (e.g., as shown in FIG. 8). Whereas augmenter 30 is shown secured to leaflet 12 in each of FIGS. 7, 8, and 9A-B, different clinical scenarios are illustrated and described in these figures. That is, in some implementations, the augmenter can be secured to the more or most dysfunctional leaflet, and in some cases, it can be secured to the leaflet opposite a leaflet having dysfunctional performance. To which leaflet the augmenter is secured depends on the performance of the valve during systole, and which leaflet, or which scallop or segment of that leaflet, can have the greatest need of reinforcement. In some implementations, more than one augmenter can be employed to treat a given valve.

Anchor 38 is used to connect panel 62 to leaflet 12. In some implementations, and as shown, this anchoring is such that articulatable coupling 66 is disposed between the root 13 and lip 15 of the leaflet—e.g., closer than the lip to the root. In some implementations, augmenter 30 can be anchored such that articulatable coupling 66 is disposed at an annulus of valve 10. In some implementations, the augmenter 30 can be of a size such that panel 62 covers most of leaflet 12. In some implementations, augmenter 30 can cover only a region close to lip 15 of leaflet 12, and/or can cover only one scallop of the leaflet.

In some implementations, the first and second panels are the same size and shape (e.g., as shown herein above). In some implementations, either panel can be larger than the other in at least one dimension, e.g., such that a lip 63 of panel 64 opposite the hinge extends beyond the opposing lip 63 of panel 62, as shown in FIGS. 9A-B.

Panel 62 can be flush with the surface of first leaflet 12, and can comprise a material configured (e.g., sufficiently flexible) to follow the contours of the first leaflet. In some instances, the first panel conforms to the changing form of the first leaflet as it opens and closes during diastole and systole.

In some implementations, augmenter 30 is provided with anchor(s) 38 pre-attached to panel 62, e.g., as shown.

In some implementations, anchor(s) 38 are provided as discrete elements to be introduced (e.g., coupled to panel 64) during implantation. The anchor is designed to attach or to be coupled to the first panel, generally near articulatable coupling 66, or at least closer to the articulatable coupling than to lip 63.

In FIG. 7, system 22 is shown treating a valve 10 of heart 4 of a subject, the valve having a dysfunction, e.g., such as that shown in FIG. 1B in which leaflet 14 fails to coapt normally with leaflet 12. During systole, augmenter 30 bridges a potential gap between leaflets 12 and 14, such that leaflet 14 contacts panel 64 of augmenter 30. During systole, augmenter 30 is in an open or expanded configuration, such that opening 40 faces ventricle 8, panel 62 is attached to leaflet 12, and panel 64 coapts with leaflet 14. During ventricular diastole, shown in the lower image of FIG. 7, as leaflets 12 and 14 separate (allowing blood into the ventricle 8), panels 62 and 64 of augmenter 30 collapse toward each other, minimizing the opening between them, and thereby reducing obstruction of blood flow through valve 10 (e.g., compared to if the augmenter were to remain in its expanded state).

FIG. 8 shows augmenter 30C having been implanted such that, at least during ventricular systole, third panel 65 becomes disposed against leaflet 14. Opening 40 faces ventricle 8, e.g., both during ventricular systole and during ventricular diastole. During ventricular systole, represented in the upper image of FIG. 8, panel 64 of augmenter 30 coapts with the upstream surface of leaflet 14. Coaptation closes the opening between leaflets 12 and 14, preventing backflow of blood from ventricle 8 into atrium 6. During ventricular diastole, shown in the lower image of FIG. 8, as leaflets 12 and 14 separate (allowing blood into the ventricle 8), panels 62 and 64 of augmenter 30 collapse toward each other, minimizing the opening between them, and thereby reducing obstruction of blood flow through valve 10 (e.g., compared to if the augmenter were to remain in its expanded state).

Reference is now made to FIGS. 9A and 9B, showing additional and separate features present in some implementations. Augmenter 30 can further comprise a tether 70 made of a metal wire, suture, fabric thread, or other connecting filament configured to tether panel 62 to ventricular tissue 80. The tether 70 is coupled to and extends from panel 62 and is attached to ventricular tissue 80 via a ventricular anchor 74, while anchor 38 anchors panel 62 to leaflet 12. Anchor 74 can anchor tether 70 to a papillary muscle, ventricular wall, interventricular septum, or other tissue of the ventricle. Tether 70 can be attached to lip 63 of panel 62, or elsewhere on panel 62.

In some implementations, the tether may advantageously stabilize the augmenter by regulating the movement of the first panel 32 with respect to the first leaflet 12, providing additional support to the augmenter. The tether can be used to stabilize or further anchor the augmenter in place, e.g., in the case of a torn chordae tendinea. Tether 70 is configured to increase the efficiency of the valve leaflet augmenter in preventing regurgitation through the valve.

Reference is now made to FIGS. 10A-C and FIG. 11, which are schematic illustrations of an augmenter 130 (e.g., a leaflet augmenter, leaflet augmenter, etc.) in accordance with some implementations. FIG. 10A shows a variant 130A of augmenter 130, and FIGS. 10B-C show a variant 130B of augmenter 130.

Augmenter 130 comprises three panels, 52, 54, and 56, arranged around an axis 82 (e.g., a central axis) of the augmenter. In some implementations (e.g., as shown in FIG. 10B), each panel substantially defines a respective plane 84 that is parallel to axis 82. In some implementations, each panel can be somewhat curved (e.g., as shown in FIG. 10C). Each panel can be substantially circular or elliptical in shape. In some implementations, each panel can be substantially rectangular in shape. The panels can include a cell growth-inducing substance. The panels can include a cell growth-inhibiting substance. Anchor(s) 58 are used to anchor panel 52 to a valve leaflet. The anchors 58 can be coupled to panel 52 closer to the edge of the panel than to axis 82 of the augmenter.

Variant 130A illustrates that, in some implementations, panels 52, 54, and 56 can be distributed at 120-degree angles around axis 82, and/or can diverge at 120 degree-angles from each other. Variant 130B illustrates that, in some implementations, two of the panels are oriented closer to each other, e.g., such that the angle between panels 54 and 56 is greater than 120 degrees. In FIG. 10C, augmenter 130B is shown substantially enface, such that axis 82 extends through the two-dimensional surface of the image, e.g., into and out of the plane of the page or surface on which the image is drawn.

In some implementations, aspects of one or more of the variants of augmenter 30 (e.g., panel composition, panel size, panel shape, panel reinforcement, and panel anchoring) can be applied to augmenter 130. For example, the composition, support, and stiffness of panels 52, 54, and 56 can be similar to that of panels 62/32, and 64/34, mutatis mutandis. In some implementations, augmenter 130 can comprise a plastic or flexible substance, e.g., a silicone or a polymer, which is shaped to define the first panel, the second panel, and the third panel. The material properties of the substance can be such that the panels can be folded or pushed together for delivery.

In some implementations, augmenter 130 lacks a hinge mechanism, such that the augmenter essentially retains its three-dimensional shape after implantation during both diastole and systole. Nonetheless, augmenter 130 can be compressed for transluminal delivery, e.g., with two or more of its panels closer to each other (e.g., in contact with each other), and configured to automatically assume the configuration shown upon deployment in the heart.

In FIG. 11 is shown schematic illustrations of a system 120, in accordance with some implementations, showing augmenter 130 implanted in the heart. First panel 52 is anchored to first leaflet 12 by at least one anchor 58. Anchor 58 can be as described for anchor 38, mutatis mutandis. Anchor 58 can be coupled to panel 52 closer to the edge of the panel than to axis 82 of the augmenter. When first panel 52 is anchored to first leaflet 12, it may or may not conform to the shape of the leaflet. In some implementations, panel 52 can be anchored closer to root 13 of leaflet 12. When panel 52 is anchored to leaflet 12, panels 54 and 56 retain their substantially parallel relationship to axis 82.

Panel 52 can be anchored to leaflet 12 such that panel 56 extends toward ventricle 8, e.g., overhanging leaflet 12. In some cases, part of panel 56 is in contact with leaflet 12.

The upper image of FIG. 11 represents valve 10 of heart 4 during systole, and the lower image represents the valve during diastole. Features described in FIGS. 7, 8, and 9A-B can be incorporated into implementations shown in FIG. 11. Augmenter 130 can be attached to leaflet 12, or in some implementations, to leaflet 14. Either leaflet 12 or leaflet 14 could be a healthy leaflet or a dysfunctional leaflet. During systole, augmenter 130 bridges a potential gap between leaflets 12 and 14, such that panel 54 and/or panel 56 coapts with leaflet 14.

In some implementations, panel 54 and panel 56 collectively define a face, such that after anchoring of panel 52 to leaflet 12, the face is presented as a coaptation surface to leaflet 14 during ventricular systole.

Anchor 58 can be configured to anchor panel 52 to leaflet 12 such that, during ventricular systole, panel 56 is disposed in ventricle 8 and panel 54 is disposed in atrium 6, e.g., panel 54 extends, within atrium 6, away from leaflet 12 and across an upstream surface of leaflet 14. Thus, during ventricular systole, panel 54 can be disposed against an upstream surface of leaflet 14. In some cases, a lip 17 of leaflet 14 contacts panel 56 during ventricular systole. During ventricular diastole, both panel 56 and panel 54 are typically disposed in ventricle 8.

System 120 can further comprise a tether (not shown; as in FIGS. 9A-B) coupled to and extending from, e.g., panel 52 or panel 56, and configured to tether the panel to ventricular tissue of the heart. The system/apparatus can further comprise a ventricular anchor, coupled to the tether, and configured to anchor the tether to the ventricular tissue. The ventricular tissue can be, e.g., tissue of a wall of the ventricle, or tissue of a papillary muscle. The ventricular anchor can be coupled to the tether in a manner that facilitates adjustment of tension on the tether. A winch can be used to couple the ventricular anchor to the tether, such that the tension on the tether is adjustable by actuation of the winch. The tether is configured to increase the efficiency of the valve leaflet augmenter in preventing regurgitation through the valve.

System 120 can further comprise a delivery tool for delivering the augmenter 130 to the heart 4. The delivery tool can anchor the first panel to the first leaflet while the second panel and the third panel are disposed within the delivery tool. Subsequently, the second panel and the third panel can be deployed from the delivery tool.

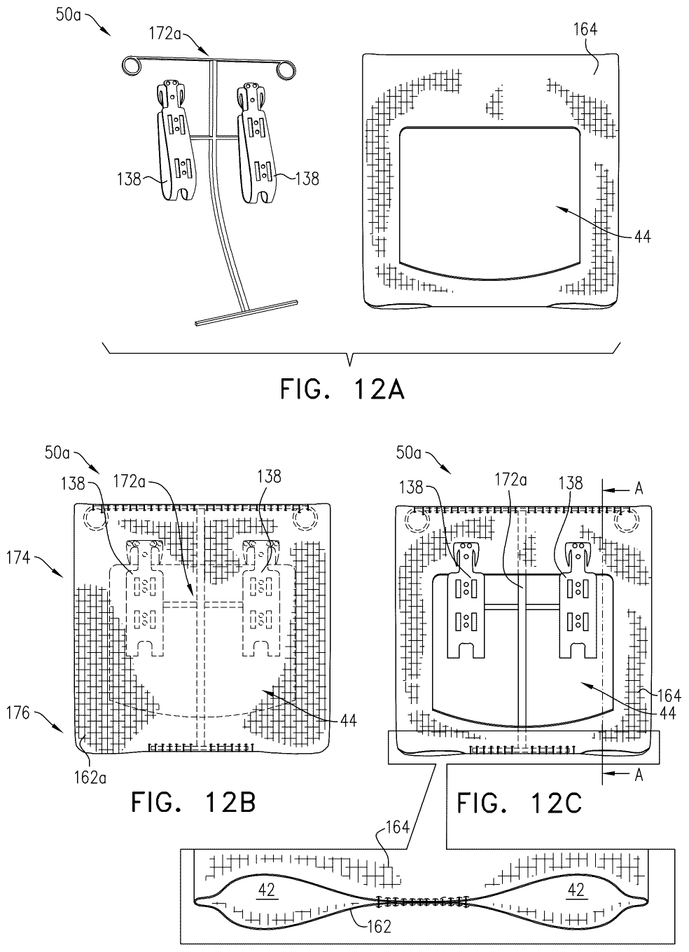

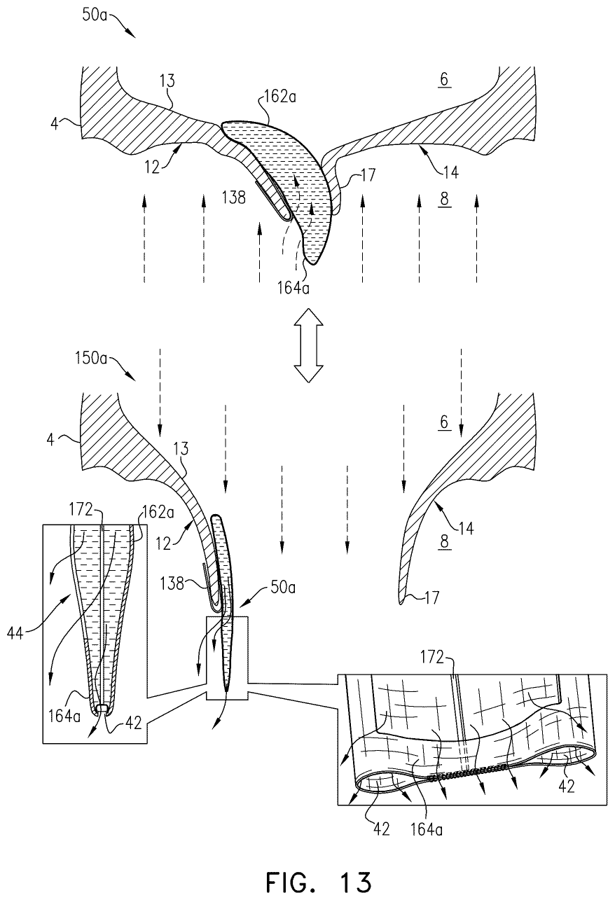

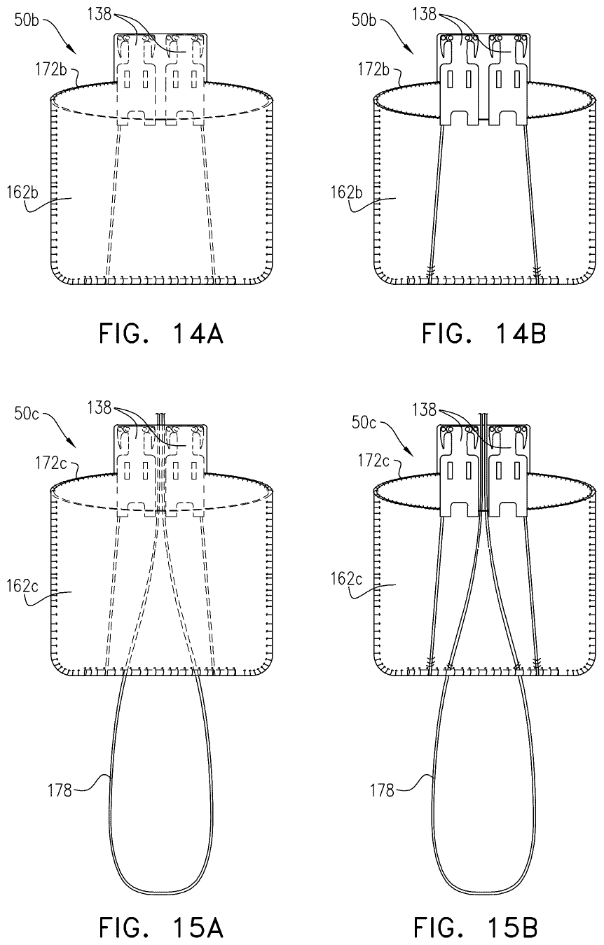

Reference is now made to FIGS. 12A-21, which are schematic illustrations of various examples of an augmenter 50 (e.g., leaflet augmenter, leaflet augmenter, etc.) and system 150, in accordance with some implementations. FIGS. 12A-C, 14A-B, 15A-B, 17 and 19A-D show leaflet augmenter 50 (variants 50a, 50b, 50c, 50d, 50e thereof), and FIGS. 13, 16, 18, and 20 show variants of system 150 (150a, 150c, 150d, 150e thereof) in which augmenter 50 (e.g., a variant thereof) is implanted at valve 10, in accordance with some implementations.

In some implementations, augmenter 50 is configured to be attached to a first leaflet 12 of valve 10 of a heart 4.

In some implementations, the augmenter comprises a frame 172 (172a, 172b, 172c, 172d, 172e), a flexible panel 162 (162a, 162b, 162c, 162d, 162e) attachable to frame 172, and an anchor 138 (138e) coupled to the frame at a coupling point. Augmenter 50 can be configured to anchor the frame to leaflet 12 such that, during ventricular systole, closure of valve 10 is accompanied by coaptation of panel 162 with second leaflet 14.

As mentioned hereinabove, first leaflet 12 of valve 10 can have a root 13, root 13 being attached to an annulus of the valve. In some implementations, anchor 138 is configured to anchor panel 162 to first leaflet 12 closer to root 13 than to a lip 15 of the leaflet. In some implementations, anchor 138 is configured to anchor panel 162 to first leaflet 12 closer to lip 15 than to root 13. In some implementations, anchor 138 is configured to anchor panel 162 to first leaflet 12 equidistant between lip 15 and root 13. In some implementations, augmenter 50 can be secured to either a healthy leaflet, or to a dysfunctional leaflet.

In some implementations, panel 162 is a first panel extending over a first side of the frame, and the augmenter further comprises a second panel 164. An example of this is augmenter 50a, shown in FIGS. 12A-C and 13. In some implementations, panel 164 can extend over an opposite side of frame 172 from panel 162. Thus, the augmenter can be configured to inflate like a pillow, and to act as a coaptation element, such as a spacer, gap filler, etc., that can coapt and/or press against one or both leaflets.

Panels 162 and 164 can be manufactured in various ways. In some implementations, the panels are assembled. In some implementations, the panels are molded. In some implementations, the panels are cut (e.g., laser cut). In some implementations, two sheets are attached at their edges, one being a complete sheet and the other having a central opening in it. In some implementations, a single sheet is folded around both sides of the augmenter (e.g., a frame or other component thereof), leaving an opening on the second side of the sheet.

Various shapes of the substantially central opening in panel 164 are possible (e.g., square, circle, rectangular, rounded, triangular, oval, etc.). In some implementations, a rounded square central opening can be used, which provides structural/functional benefits, e.g., the edges of the opening are somewhat self-supporting and can accommodate the force of inflation without flapping open. The shape of the opening may be of greater importance at a lip portion 176 of the augmenter and may be less important at a root portion 174 of the augmenter.

In some implementations, panels 162 and 164 can be attached to each other along at least one edge, e.g., so as to define a cavity that can accept blood during systole, e.g., that can billow. Second panel 164 can have a central opening 44. In some implementations, opening 44 is larger than a closed part of the second panel. That is, panel 164 can be more open than closed, e.g., such that the closed part resembles an open hem. Opening 44 can have straight edges, and/or rounded corners, e.g., can be in the shape of a rounded square or rounded rectangle.

As mentioned, in some implementations, augmenter 50 can be configured to have a root portion 174 at one end, and a lip portion 176 at an opposite end of the augmenter, such that anchor 138 is configured to anchor augmenter 50 to lip 15 of first leaflet 12 such that the lip portion is downstream of the root portion.

In some implementations, and as shown in FIGS. 12C and 13, at one edge of the lip portion, panels 162 and 164 are unattached such as to define one or more opening(s) 42 that, once augmenter 50a is implanted, face toward the ventricle and/or are disposed within the ventricle.

As shown in the left inset of FIG. 13, corresponding to line A in FIG. 12C, openings 42 can provide passage for blood, e.g., facilitating exiting of blood from between panels 162 and 164 during diastole.

In any variants of augmenter 50, the sheets comprising panels 164 and 162 can comprise a biocompatible material, e.g., a film, a fabric, a polymer, a metal, etc. The fabric can comprise, e.g., a woven polymer or knitted polymer.

In some implementations, and as shown in FIGS. 12A-18, panel 162 is attached to the frame in a manner that enables the panel (e.g., the sheet thereof) to billow in response to hemodynamic forces, such that billowing of the panel enhances coaptation of the augmenter with a second leaflet 14 of the valve during systole, e.g., the panel billows toward one or both leaflets.

In some implementations, rather than having the sheet of the panel 162 (164) tightly bound to frame 172, the sheet is attached only at specific points, allowing the panel(s) to billow like a sail, and thereby to provide better coaptation with the opposing leaflet. In some implementations, panel 162 is attached only at one or more edges of frame 172.

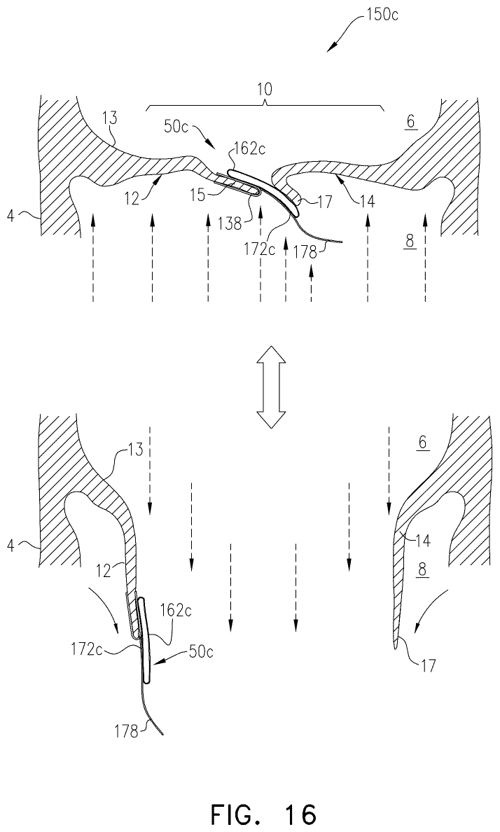

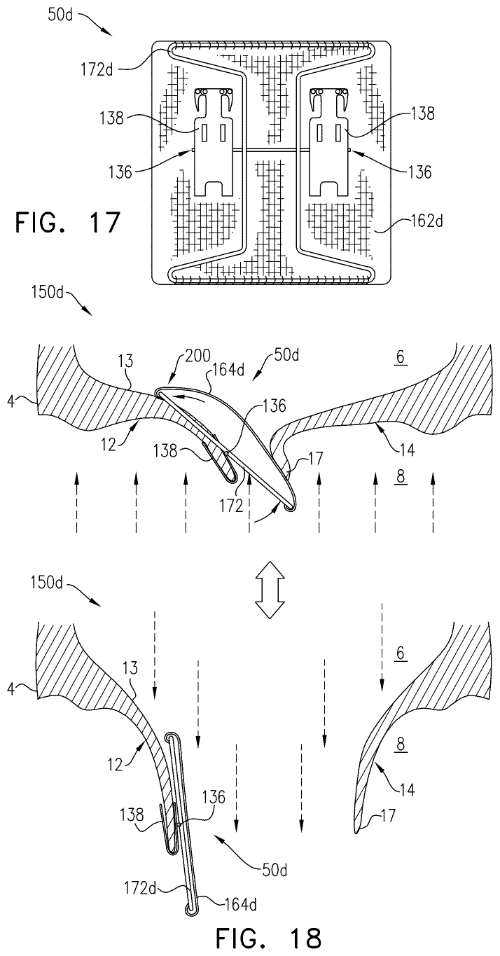

In some implementations, e.g., as shown in FIGS. 14A-16, the attachment may be all the way around a perimeter of frame 172. In some implementations, e.g., as shown in FIGS. 12A-13 and 17-18, the attachment may be only at the root and the lip of the augmenter.

In some implementations, anchor 138 is coupled to a central portion of frame 172. In some implementations, the anchor is coupled to a proximal portion of the frame. In any variants of augmenter 50, the anchor can be, e.g., a single clip, or a pair of clips.

In some implementations, anchor 138 is pivotably coupled to frame 172, as shown, e.g., in FIGS. 17-18, such that augmenter 50 is configured to pivot with respect to the first leaflet responsively to fluid pressure.

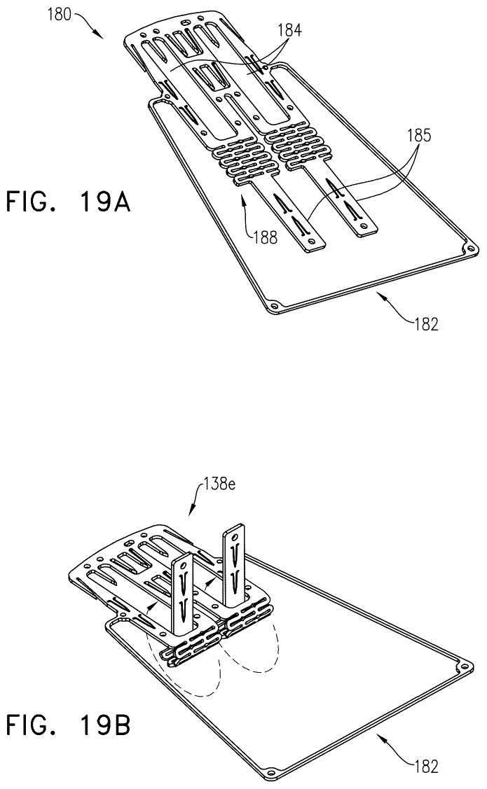

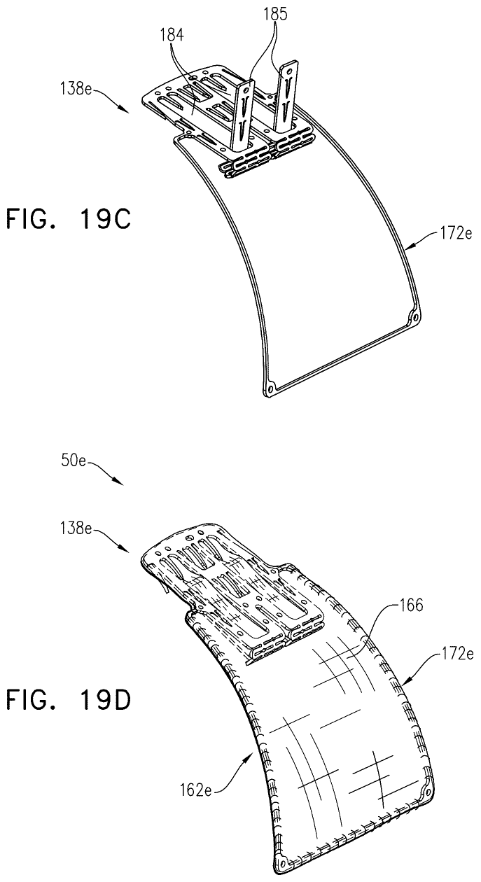

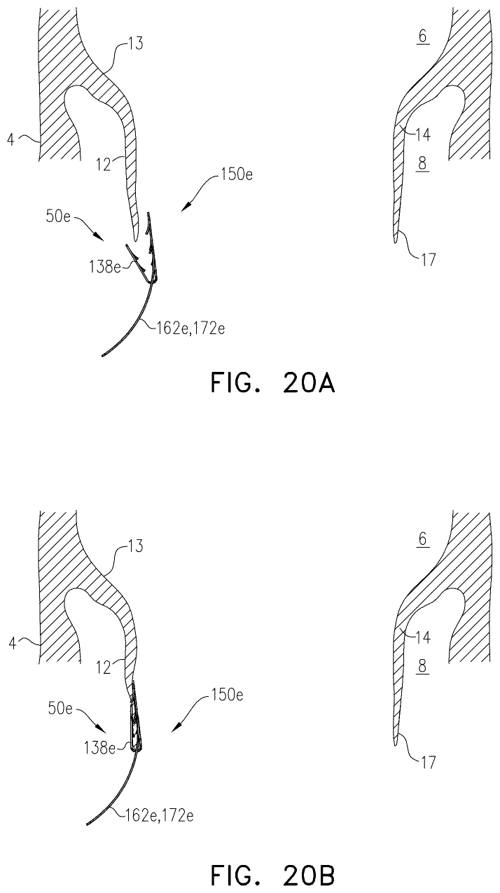

In some implementations, anchor 138 is fixedly coupled to frame 172, and can even be cut from the same piece of stock material as the frame, as shown in FIGS. 19A-21.

In any variants of augmenter 50, frame 172 can comprise a shape-memory metal.

In some implementations, and as shown in FIGS. 15A-B, frame 172c comprises a tongue 178, e.g., a loop of wire, extending distally beyond the panel, the tongue configured to stabilize coaptation of the augmenter with second leaflet 14.

In some implementations, the size of the tongue can be adjustable, e.g., via one or more free ends of the loop that are accessible proximally from the augmenter. As shown in FIG. 16, tongue 178 is positioned to tuck underneath the opposing leaflet during systole, advantageously providing additional stability and reducing the chance of the augmenter flailing into the atrium. The tongue may allow the augmenter itself to remain flexible and may also reduce the possibility of trauma to the opposing leaflet and/or its chordae.

In some implementations, and as shown in FIGS. 17-18, anchor 138 is pivotably coupled to a central region of frame 172, the pivotable coupling 136 enabling augmenter 50 (e.g., variant 50d) to exhibit a see-sawing motion with respect to first leaflet 12, such that the see-sawing motion enhances coaptation of the augmenter with lip 17 of second leaflet 14. In some implementations, the anchors 138 of the augmenter are disposed toward a central region of the augmenter rather than at a root region, and are hingedly coupled to the frame itself, thus allowing the augmenter to see-saw. This configuration advantageously enables the root region of the augmenter to be closer to the root of the leaflet to which the augmenter is anchored compared to implementations in which the anchors are at the root of the augmenter. This may advantageously reduce the chance of leakage between the augmenter and the leaflet to which it is anchored. The hinged coupling may advantageously augment this effect.

For example, when the lip of the augmenter is pushed in an upstream direction by ventricular blood during systole, the augmenter pivots about its anchors at pivotable coupling 136, pressing the root of the augmenter against the upstream surface of the leaflet to which the augmenter is anchored (indicated by reference numeral 200 in FIG. 18), and thereby reducing a likelihood of retrograde leakage therebetween.

In some implementations, e.g., as shown in FIGS. 19A-20, augmenter 50 (e.g., augmenter 50e) comprises a unitary skeleton 180, cut from a sheet of shape-memory alloy to define a frame-region 182 and an anchor-region 188 (FIG. 19A).

In some implementations, the frame-region can be heat-set to define a frame 172e, and the anchor-region can be heat-set to define an anchor 138e.

In some implementations, a sheet 166 can be attached to skeleton 180 (e.g., to frame 172e) in a manner that defines a panel 162e. In some implementations, anchor 138e can be configured to anchor augmenter 50e to first leaflet 12 such that panel 162e coapts with second leaflet 14 during systole.

In some implementations, sheet 166 can extend further along skeleton 180 to cover anchor-region 188 of the skeleton in which anchor 138e is disposed, so that the opposite leaflet 14 contacts only sheet 166 (e.g., rather than a metal component).

In some implementations, the augmenter 50e (e.g., as shown in FIGS. 19A-20) serves as an extension of a valve leaflet, to increase coaptation, being secured to leaflet 12 with anchor 138e that is defined by skeleton 180 of the augmenter.

In some implementations, anchor 138e comprises a set of clips, e.g., shaped as legs 185. In some implementations, the legs are heat-set to have a resting position that is displaced from that in which they resided when in the original plane of skeleton 180, e.g., displaced by at least 90 degrees (e.g., at least 180 degrees) and/or no greater than 360 degrees. In the example shown, the legs' resting position is displaced by approximately 270 degrees from the original plane of skeleton 180 (FIG. 19B). In the absence of sheet 166, and as shown in FIG. 19B, the legs of anchor 138e can extend through frame 172e, e.g., through openings 184 in the frame. Once sheet 166 has been attached, the sheet may inhibit the clip legs from extending through frame 172e, e.g., the legs may press against the sheet (FIG. 19D). Thus, in some implementations, the augmenter 50e can be configured to anchor to leaflet 12 by sandwiching the leaflet between sheet 166 of panel 162e and the legs 185.

As mentioned hereinabove, the frame 172e and anchor 138e of augmenter 50e can be configured with two shape-set regions. In some implementations, the method for manufacturing the augmenter comprises cutting, from a sheet of a shape memory alloy, a unitary skeleton 180 that includes a frame-region 182 and an anchor-region 188 (FIG. 19A). In a first heat-setting procedure, the anchor-region 188 is heat-set to define an anchor 138e (FIG. 19B). In a second heat-setting procedure, the frame-region 182 is heat-set to define a frame 172e, e.g., having a curvature to facilitate coaptation with the opposing leaflet (FIG. 19C). By attaching a sheet 166 to the frame 172e, a panel 162 is formed (FIG. 19D). Anchor 138e can be configured to anchor augmenter 50e to the first leaflet such that panel 162e coapts with second leaflet 14 during systole.

In some implementations, further details of the manufacturing process may be included. In some implementations, the first shape-setting step bends the legs of anchor-region 188, from being flat within the plane of skeleton 180, at least 180 degrees, and in some implementations to 270 degrees, to form the clip structure of anchor 138e. In some implementations, the frame-region 182 can be shaped to define openings 184, and the shape-setting of legs 185 can configure them to pass through the openings, beyond the plane of the skeleton in the region of the openings, in order to provide high clamping forces. As noted above, once openings 184 are covered by sheet 166, the sheet can limit the extent to which the legs can extend beyond the plane of the skeleton. In some implementations, and as shown, legs 185 are separate from each other. In some implementations, legs 185 can have connecting struts extending therebetween, which can inhibit the legs from passing through openings 184, e.g., alternatively to or additionally to the inhibition provided by sheet 166.

In some implementations, the heat-setting that defines anchor 138e is performed using a first mold or jig, and the heat-setting procedure that defines frame 172e is performed using a second mold or jig. An example manufacturing technique can include: (i) constraining anchor-region 188 in the first mold or jig, (ii) heat-setting the anchor-region while it remains thus constrained (e.g., by placing skeleton 180 and the first mold or jig in an oven), subsequently (iii) constraining frame-region 182 in the second mold or jig, and (iv) heat-setting the frame-region while it remains thus constrained, e.g., by placing skeleton 180 and the second mold or jig in the oven. In some implementations, anchor 138e, e.g., the heat-set anchor-region 188, is supported (e.g., restrained) in its heat-set shape during the heat-setting of frame-region 182 so that heat-set shape of the anchor region is not altered during this second heating. In some implementations, this support is provided by the second mold or jig, e.g., the heat-set anchor-region 188 can be removed from the first mold or jig and placed in the second mold or jig prior to the heat-setting of frame-region 182.

Reference is made to FIGS. 20A-B, illustrating possible steps in the implantation of augmenter 50e. Augmenter 50e can be delivered to the area of the valve to be augmented via any number of approaches, e.g., transcatheter via the vasculature, or transapical. System 150e can define a delivery state of augmenter 50e, in which anchor 138e is held in an open configuration, e.g., legs 185 are restrained to prevent their passage through openings 184, until the lip of leaflet 12 is disposed within the anchor (FIG. 20A). Legs 185 are then released from this restraint, such that anchor 138e transitions toward its heat-set shape (e.g., closes), thereby securing the augmenter to the lip of the leaflet (FIG. 20B).

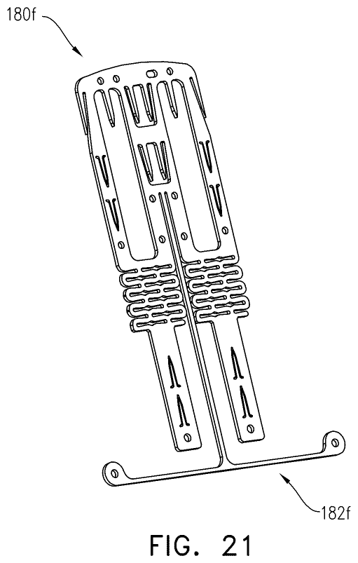

Frame 172e is shown as running along (e.g., defining) the perimeter of panel 162e. However, other frame shapes are possible. For example, FIG. 21 shows a skeleton 180f that has a frame-region 182f that, once heat-set, will define a T-shaped frame, e.g., including a spine that runs along a centerline of the panel.

Reference is again made to FIGS. 1A-21. Several possible advantages of the designs shown in the present application may exist. For example, augmenters 30, 130, and 50 require attachment to only a single valve leaflet. This may provide the possibility of adapting or scaling the size of the augmenter to an optimal size and shape for the valve being treated. In addition to being suitable for valves having incomplete coaptation, implementations of the present system may be applicable to treatment of annular calcification treatment, as no annular contraction is required. Some implementations of the augmenter may have advantages in transluminal insertion as the design allows the panels to be shorter or smaller than a comparable single-panel augmenter. Although an optional tether is described herein above, it is to be noted that the augmenters described herein advantageously do not require a ventricular tether.

Reference is again made to FIGS. 1A-21. It is therefore to be noted that, in some implementations, an augmenter (50) is provided that comprises a frame (172), an anchor (138), and a panel(s) (162, 164) that comprises a flexible sheet (166). The anchor is coupled to the frame and is configured to anchor the augmenter to the lip of a first leaflet of a valve of a heart. The sheet can have a first surface that faces the frame and a second surface that faces away from the frame. The sheet can be coupled to the frame in a manner that provides a billow zone at which the sheet is unattached to the frame, thereby facilitating the panel billowing away from the frame responsively to fluid pressure at the first surface.

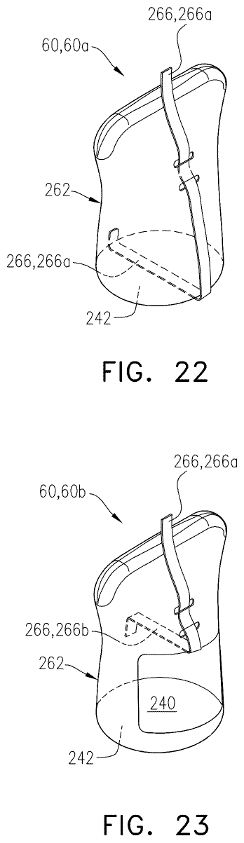

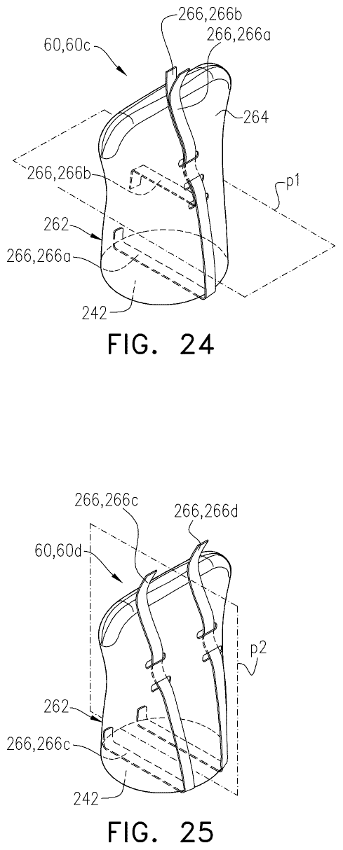

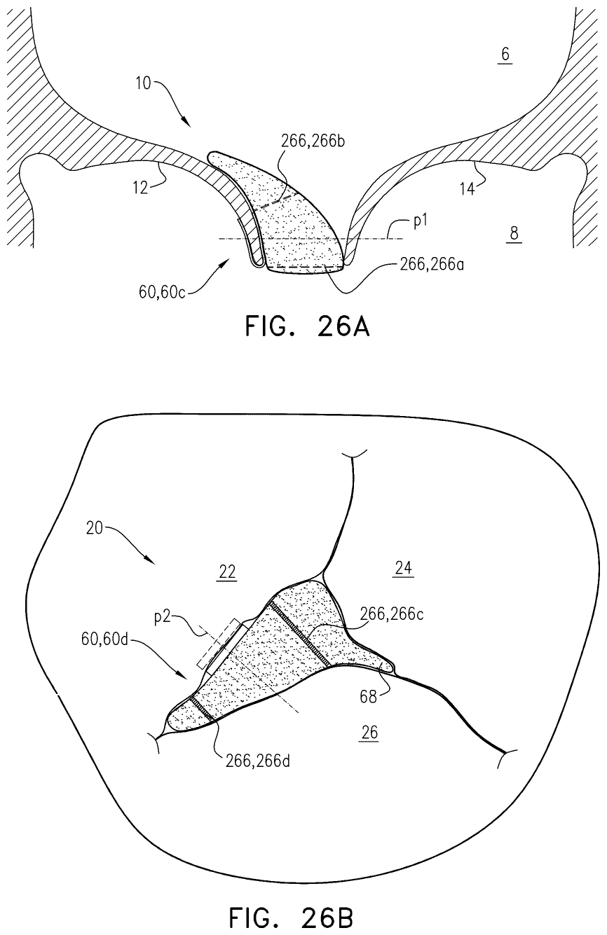

Reference is now made to FIGS. 22-25 and to FIGS. 26A-B, which are schematic illustrations of a leaflet augmenter 60 (e.g., variants thereof), in accordance with some implementations. FIG. 22 shows an augmenter 60a, FIG. 23 shows an augmenter 60b, FIG. 24 shows an augmenter 60c, FIG. 25 shows an augmenter 60d, the suffixes indicating that these are respective variants of augmenter 60. FIGS. 26A-B show augmenter 60 implanted at a valve 10 (e.g., a mitral valve) or a valve 20 (e.g., a tricuspid valve), in accordance with some implementations.

Augmenter 60 comprises a pouch 262 having an internal dimension and a downstream opening 242. Once anchored to a valve, downstream opening 242 of pouch 262 enables augmenter 60 to inflate during systole, such that an external surface of the pouch presses against leaflets of the valve, the pouch thereby closing gaps between the leaflets of the valve during systole, thus diminishing regurgitation in a valve having insufficiency.

Augmenter 60 comprises one or more anchor(s), coupled to the pouch, and configured to anchor the augmenter to a leaflet of the valve. The anchor can comprise, e.g., tissue-piercing elements, T-bars, threads, sutures, pins, clips, or locking elements. The anchor can be as described for any of the anchors disclosed herein. For the sake of clarity, the anchor is omitted from FIGS. 22-25.

In some implementations, augmenter 60 comprises at least one adjustable strap 266 (266a, 266b, 266c, 266d), coupled to pouch 262 such that adjustment of the strap (e.g., adjustment of its length) adjusts the internal dimension of the pouch. Strap 266 can be slidably coupled to part of the pouch (e.g., can be threaded through part of the pouch), and/or fixed to part of the pouch.

In some implementations, strap 266 spans downstream opening 242. An example of this is strap 266a of augmenter 60a, shown in FIG. 22.

In some implementations, strap 266 spans the interior of pouch 262. An example of this is strap 266b of augmenter 60b, shown in FIG. 23.

In some implementations, augmenter 60 comprises multiple straps, enabling finer control of the size of downstream opening 242. In some such implementations, variants of straps 266 can be disposed at different positions with respect to pouch 262. For example, augmenter 60c (FIG. 24) comprises both strap 266a and strap 266b. Another example is augmenter 60d (FIG. 25), which comprises straps 266c and 266d.

In some implementations, and as shown for augmenter 60c, the straps can be disposed on opposite sides of a transverse plane p1 of the pouch. The straps can lie parallel with plane p1 and/or with each other. Plane p1 can lie parallel to downstream opening 242. In some implementations, and as shown for augmenter 60d, the straps can be disposed on opposite sides of a longitudinal plane p2 of the pouch. The straps can lie parallel with plane p2 and/or with each other. Plane p2 may bisect downstream opening 242.

Although FIGS. 22-25 do not show the anchor of augmenter 60, the anchor can be disposed on plane p1 and/or on plane p2.

In any variants of augmenter 60, the specifications of pouch 262 and arrangement of the one or more straps 266 (e.g., 266a, 266b, 266c, and/or 266d) can be selected to conform as closely as possible to the requirements of the valve to be corrected.

Adjustment of the length of strap(s) 266 is configured to enable control of the effective size and shape of the internal dimension during implantation of augmenter 60, e.g., during closure of the valve during systole. For example, lengthening strap 266 increases the internal dimension, thus potentially enhancing coaptation between the augmenter and the second leaflet during systole. Using the straps to adjust the size and shape of augmenter 60 may advantageously enable the augmenter to better suit the anatomy of the valve at which it is implanted, thus improving performance of augmenter 60 in reducing regurgitation through the valve. In some implementations, an imaging modality is employed during implantation and/or adjustment of straps 266 (variants thereof) to facilitate coaptation of the external surface against the opposing leaflet.

In any variants of augmenter 60, pouch 262 can comprise a biocompatible material, e.g., a film, a fabric, a polymer, a metal, etc. The fabric can comprise, e.g., a woven polymer or knitted polymer. Pouch 262 can be shaped as and/or comprise a rectangle, cone, rounded square, globe, pyramid, or any other shape that suits the shape of the valve to be repaired.

Pouch 262 can be manufactured in various ways. In some implementations, the pouch is molded. In some implementations, the pouch is manufactured by stitching together individual sheets. In some implementations, the pouch is manufactured by three-dimensional weaving, e.g., without requirement for stitching together of sheets.

In some implementations, e.g., as shown in FIG. 23, one side of pouch 262 can have another opening, such as on the side of the pouch on which the anchor is disposed, e.g., opening 240 or similar. Opening 240 can be configured to provide hemodynamic balance during inflow of blood to the augmenter, e.g., to prevent excessive hemodynamic pressure on the augmenter.

In any variants of augmenter 60, adjustable strap 266 can comprise, e.g., a belt, cord, line, thread, braid, thong, tape, tie, cable, wire, elastic, or ligature. Adjustable straps 266 can have different lengths, widths, and/or can comprise materials, e.g., a fabric, of different strengths and flexibility. The length of adjustable straps 266 can be adjusted by pulling on a free end of the strap. The adjusted length can be maintained by a variety of mechanisms, e.g., a ratchet, friction, hook and loop self-fastener, or other passive mechanism. In some implementations, the adjusted length can be maintained by an active locking mechanism, e.g., a buckle, a locking bead, or clamp. In some implementations, augmenter 60 itself comprises an adjustment mechanism that can be actuated in a manner that adjusts the length of strap(s) 266.

In some implementations, augmenter 60 is configured to be attached to a first leaflet 12 of valve 10 having two leaflets (e.g., a mitral valve), as shown in a frontal section of the valve in FIG. 26A. Augmenter 60, e.g., an anchor(s) thereof, can be configured to anchor pouch 262 to leaflet 12 such that, during ventricular systole, closure of valve 10 is accompanied by coaptation of pouch 262 with the single opposing leaflet, e.g., second leaflet 14.

In some implementations, augmenter 60 is configured to be attached to a first leaflet 12 of a valve 20 having three leaflets (e.g., a tricuspid valve), as shown in a view from above, in FIG. 26B. Augmenter 60, e.g., an anchor(s) thereof, can be configured to anchor pouch 262 to leaflet 12 such that, during ventricular systole, closure of valve 20 is accompanied by coaptation of pouch 262 with one of the opposing leaflets or, as shown, with both of the opposing leaflets, e.g., second and third leaflets 24 and 26. In some implementations, augmenter 60 is configured such that, responsively to ventricular systole, at least part of the pouch at 69 interposes between the first opposing leaflet and the second opposing leaflet.

As mentioned hereinabove, first leaflet 12 of valve 10 can have a root 13, root 13 being attached to an annulus of the valve. In some implementations, the anchor is configured to anchor pouch 262 to first leaflet 12 closer to root 13 than to a lip 15 of the leaflet. In some implementations, the anchor is configured to anchor pouch 262 to first leaflet 12 closer to lip 15 than to root 13. In some implementations, the anchor is configured to anchor pouch 262 to first leaflet 12 equidistant between lip 15 and root 13. In some implementations, augmenter 60 can be secured to either a healthy leaflet, or to a dysfunctional leaflet.

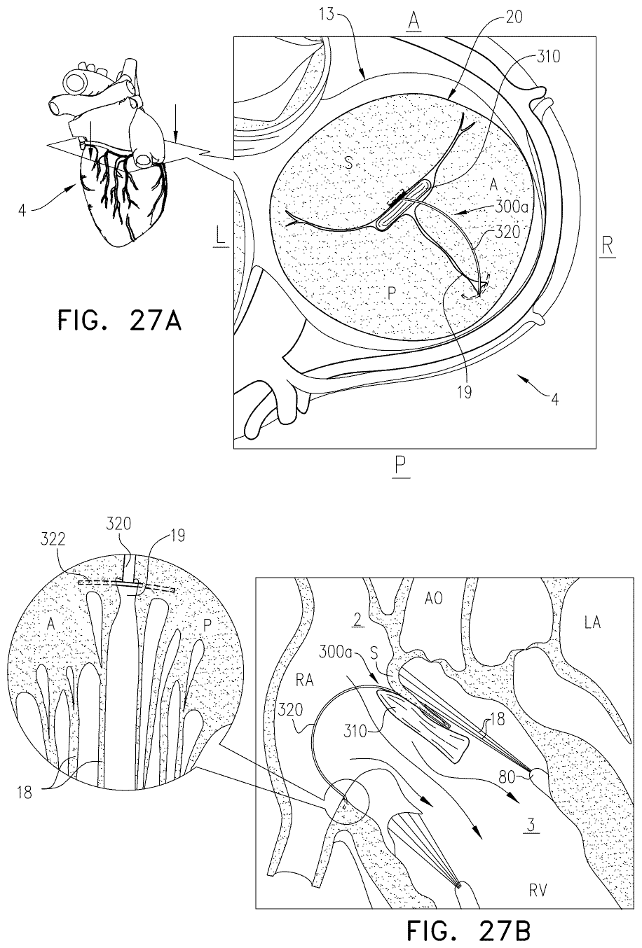

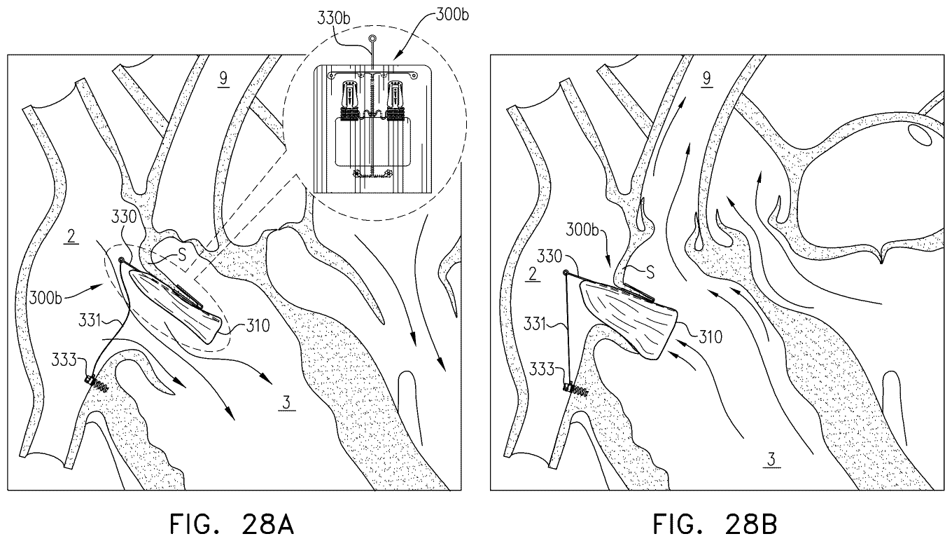

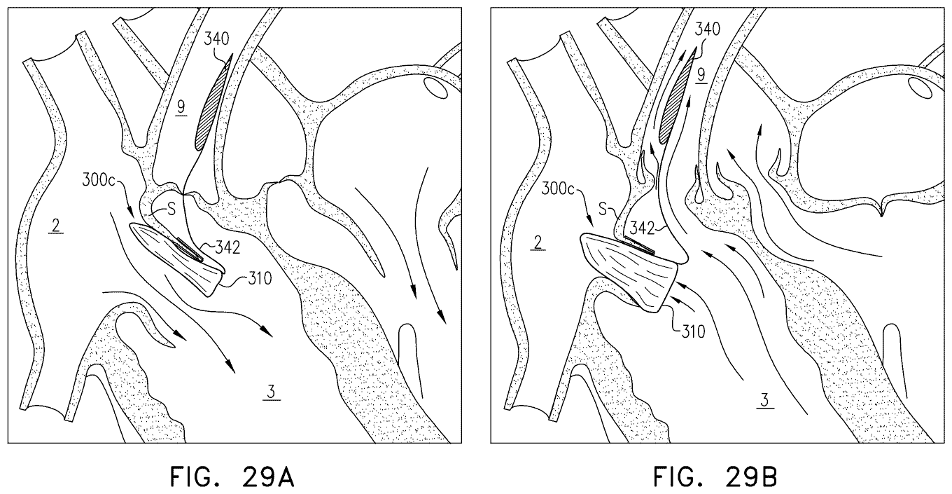

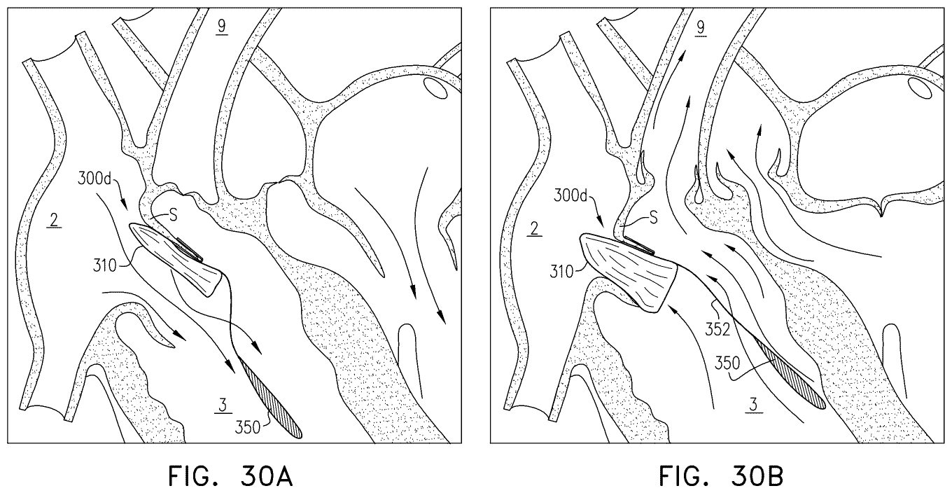

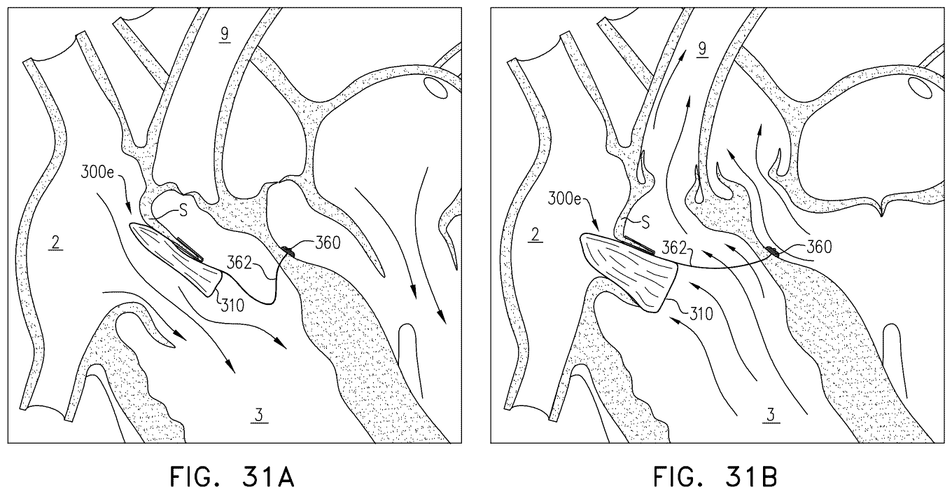

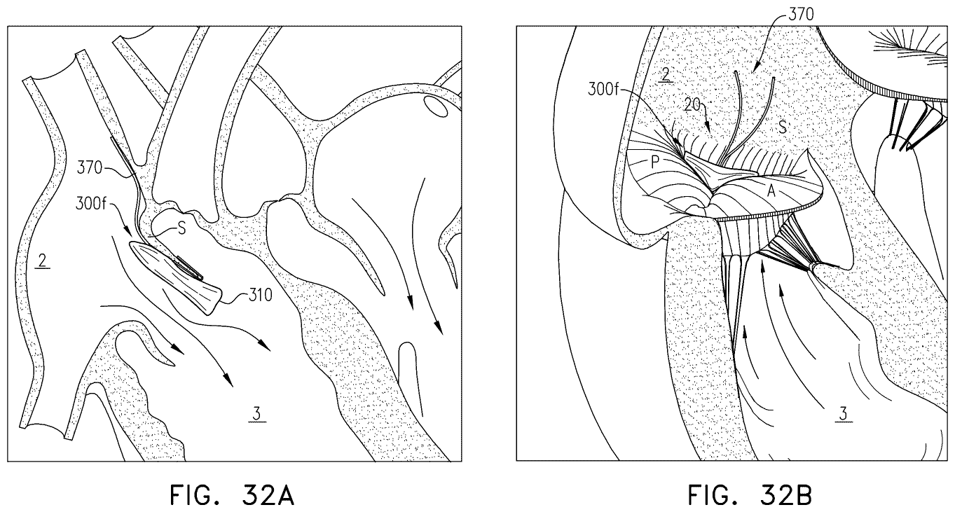

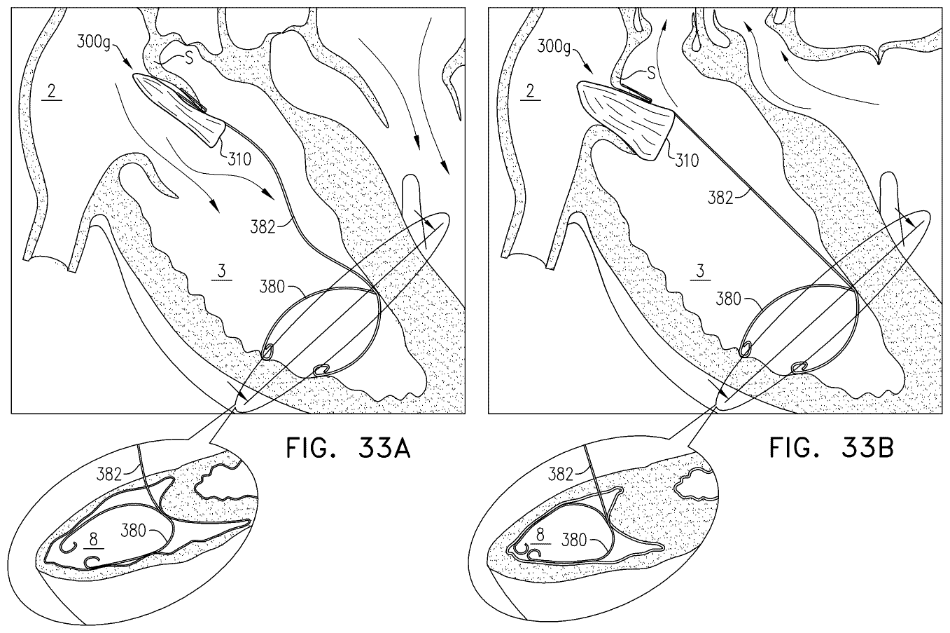

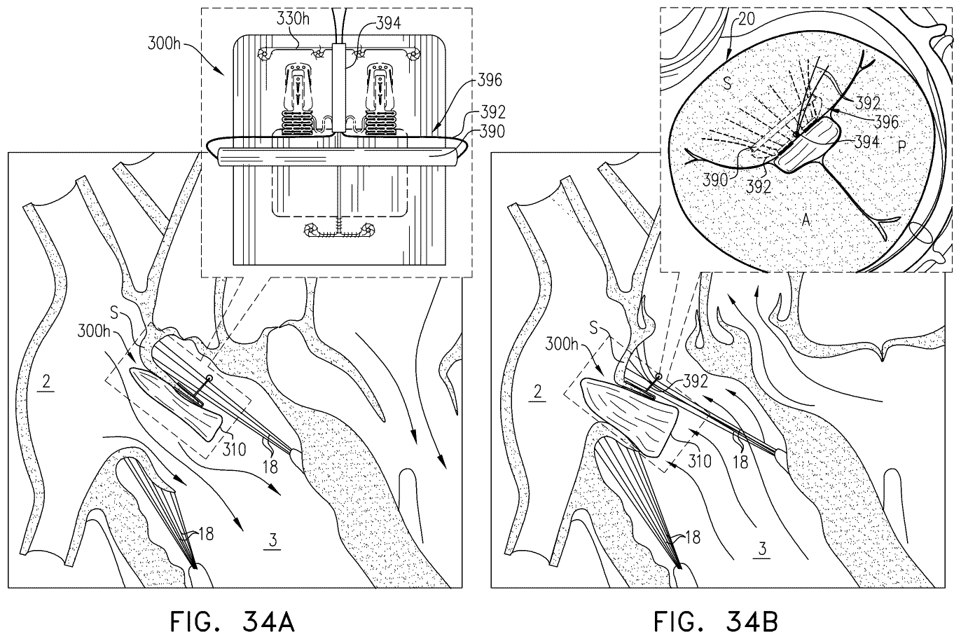

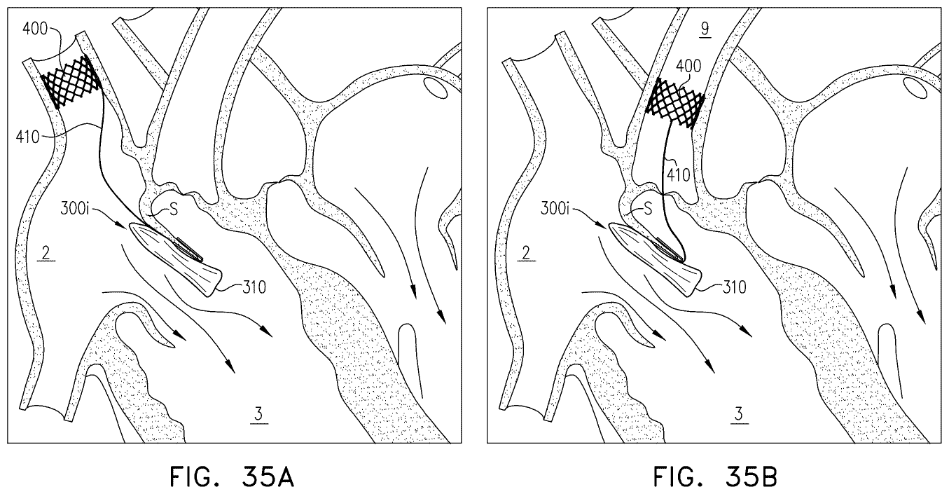

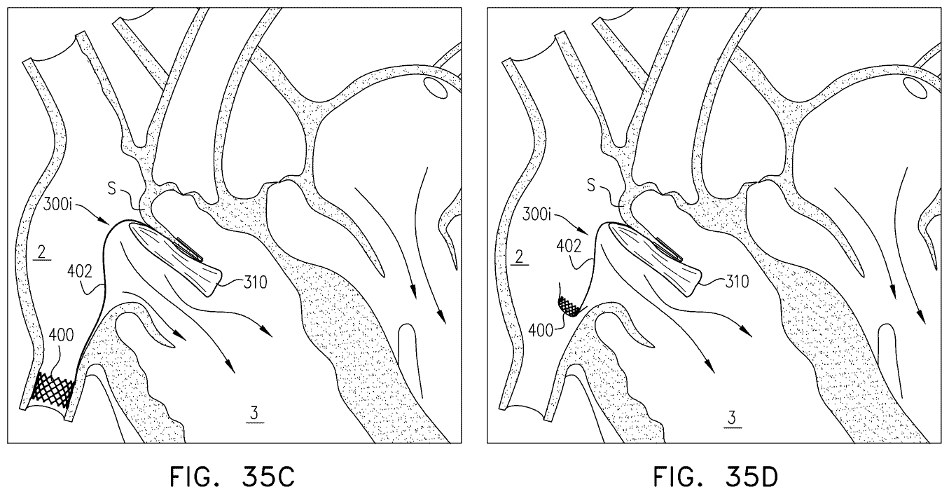

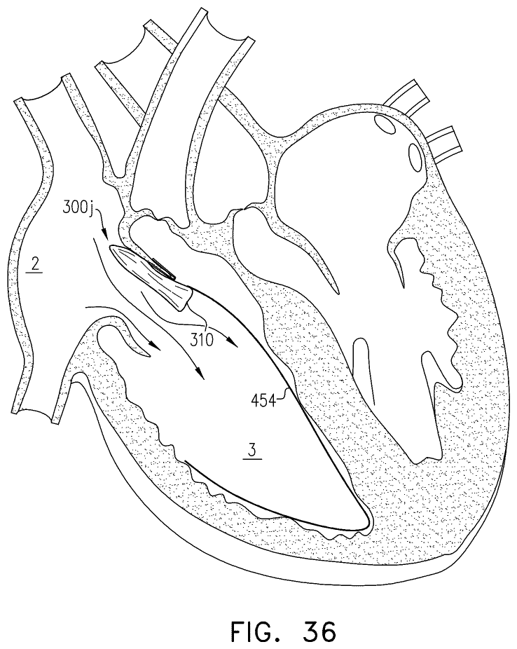

Reference is now made to FIGS. 27A-36, which are schematic illustrations of a leaflet augmenter 300 (e.g., variants thereof), implanted at a valve 20 of a heart 4, in accordance with some implementations. Augmenter 300 can share features of, or be essentially the same as, any of augmenters 30, 50, and 60, described herein above. Similarly, any of augmenters 30, 50, and 60 can be modified to include features of augmenter 300 (e.g., of any variant thereof), such as its stabilization features. In these figures, valve 20 is a valve with three leaflets, e.g., the tricuspid valve, disposed between the right atrium 2 and the right ventricle 3 of heart 4. In FIG. 27A, valve 20 is shown with the three leaflets designated as S for septal, P for posterior, and A for anterior, respectively. However, in some implementations, augmenter 300 (e.g., variants thereof) can be used at a bicuspid valve such as the mitral valve, mutatis mutandis.

In some implementations, augmenter 300 comprises a pouch 310. In some implementations, pouch 310 can be the same as or similar to pouch 262 shown in FIGS. 22-26B.

In some implementations, augmenter 300 comprises one or more anchor(s), coupled to the pouch, and configured to anchor the augmenter to a leaflet of the valve. The anchor can be as described for any of the augmenters disclosed elsewhere herein. In some implementations, the anchor comprises, e.g., tissue-piercing elements, T-bars, threads, sutures, pins, clips, clamps, or locking elements. For the sake of clarity, the anchor is unlabeled in FIGS. 27A-36.

In some implementations, augmenter 300, e.g., a variant thereof, comprises frame 330 although, for the sake of clarity, the frame is not explicitly shown in FIGS. 27A-B, 29A-33B, and 35A-36. Furthermore, augmenter 300 may not comprise frame 330, and in some implementations may not comprise a frame at all.

In some implementations, each variant of augmenter 300 comprises a respective stabilizer, e.g., an element configured to provide a stabilizing effect on the augmenter. The stabilizer can be coupled to the frame. Such a stabilizer may be particularly advantageous for implementations in which the augmenter is for treatment of the tricuspid valve. The leaflets of the tricuspid valve may provide less of a counter force, compared to those of the mitral valve, that inhibits the pouch of the augmenter from pivoting to prolapse into the atrium upstream of the valve. For example, the leaflets of the tricuspid valve may be more compliant than those of the mitral valve, and/or the coaptation line between the two leaflets to which the augmenter is not anchored may offer the augmenter a space at which to prolapse.

In some implementations, the stabilizer includes a projection, such that the stabilizer extends away from the pouch and/or the frame to the projection of the stabilizer. In some implementations, the stabilizer is configured to be positioned with the projection at a site in the heart in a manner that provides a counterforce, an anchoring effect, and/or a hemodynamic effect that resists upstream (e.g., atrial-ward) pivoting of the augmenter into the atrium during hemodynamic movements of valve 20 during the cardiac cycle.

As mentioned, FIGS. 27A-B through FIG. 36 show augmenter 300 implanted at valve 20. Once anchored to the valve, the downstream opening of pouch 310 enables augmenter 300 to inflate during systole, such that an external surface of the pouch presses against leaflets of the valve 20, the pouch thereby minimizing gaps between the leaflets of the valve during systole, thus diminishing regurgitation in a valve having insufficiency.

FIGS. 27A-B show an augmenter 300a, FIGS. 28A-B show an augmenter 300b, FIGS. 29A-B show an augmenter 300c, FIGS. 30A-B show an augmenter 300d, FIGS. 33A-B show an augmenter 300e, FIGS. 32A-B show an augmenter 300f, FIGS. 33A-B show an augmenter 300g, FIGS. 34A-B show an augmenter 300h, FIGS. 35A-D show an augmenter 300i, and FIG. 36 shows an augmenter 300j, the suffixes indicating that these are respective variants of augmenter 300.

In FIGS. 27A-B, augmenter 300a is shown implanted at valve 20, anchored to septal leaflet S. FIG. 27A shows a perspective view of heart 4, and an enlarged view of a transverse cross-section of the heart at the level of valve 20, e.g., at the annular plane.

In some implementations, augmenter 300a comprises pouch 310 and a stabilizer 320. In some implementations, stabilizer 320 comprises a bowed spring (e.g., a resilient wire) extending in an arc upstream of pouch 310. In some implementations, stabilizer 320 is configured to press (e.g., to apply a counterforce) against a commissure 19 on an opposite side of the valve from the anchor.

A projection 322 of stabilizer 320 can take various forms, such as a curved hook, T-bar, pin, clip, or clamp. In some implementations, projection 322 is configured to be placed against a downstream surface of commissure 19 between leaflets A and P of valve 20, and to maintain engagement between stabilizer 320 and the commissure. In some implementations, projection 322 can be disposed between chordae tendineae 18, as shown in magnified view in FIG. 27B.

During ventricular systole, the movement of the leaflets of the valve toward each other further bows the bowed spring, and the resistance of the bowed spring to this bowing inhibits atrial-ward deflection of the pouch.

In FIGS. 28A-B, augmenter 300b is shown implanted at valve 20, anchored to septal leaflet S. FIG. 28A represents the heart in ventricular diastole, and FIG. 28B represents the heart in ventricular systole. In some implementations, augmenter 300b comprises pouch 310, and frame 330 (e.g., variant 330b) defines a mast that can extend above the pouch.

In some implementations, stabilizer 331 comprises a tether coupled to the mast of frame 330.

In some implementations, a projection 333 of stabilizer 331 comprises an anchor, e.g., a tissue anchor, configured to be anchored into heart tissue upstream of the valve. Stabilizer 331 has a length (or can be given a length during implantation) that configures the tether to become tensioned during ventricular systole, thereby inhibiting atrial-ward deflection of the pouch.

FIGS. 29A-B and FIGS. 30A-B show augmenters 300c and 300d, respectively, implanted at valve 20, anchored to septal leaflet S. FIGS. 29A and 30A represent the heart in ventricular diastole, and FIGS. 29B and 30B represent the heart in ventricular systole. Augmenters 300c and 300d comprise stabilizer 340 and 350, respectively.

In some implementations, each of stabilizers 340 and 350 comprises a foil (e.g., a hydrofoil) that utilizes blood flow over its surface to stabilize pouch 310. In the examples shown, the stabilizer extends as a projection of the augmenter, e.g., connected to pouch 310 via an elongate connector 342. However, the stabilizers can be coupled or mounted by other means, e.g., can be more directly coupled to the pouch or to the frame of the augmenter.

In FIGS. 29A-B, stabilizer 340 (e.g., its connector 342) is configured to traverse the pulmonary valve into the right pulmonary artery 9, such that the foil is disposed within the pulmonary artery. In some implementations, a connector 342 having an adjustable or predetermined length, can be configured to provide a distance between pouch 310 and stabilizer 340.

In some implementations, the foil is configured to have a shape and an angle of attack that generates lift within pulmonary artery 9 in a manner that inhibits pouch 310 from pivoting upstream. In the example shown, this lift pushes the foil laterally across pulmonary artery 9, thereby applying a stabilizing force to pouch 310 via connector 342. For example, the lateral movement of the foil can apply a pivoting force to connector 342 and/or pouch 310 that opposes the pivoting force that can be applied to the pouch by ventricular pressure during ventricular systole.

In FIGS. 30A-B, stabilizer 350 comprises a foil configured to project into ventricle 8. In some implementations, the foil has a shape and an angle of attack that is configured to utilize hemodynamic forces within ventricle 8 in a manner that inhibits pouch 310 from pivoting upstream, in a similar manner to that described for stabilizer 340. For example, and as shown, the foil can be shaped and angled such that, during ventricular systole, the foil becomes pushed laterally toward the ventricular wall below the root of the leaflet to which augmenter 300d is anchored.

In FIGS. 31A-B, augmenter 300e is shown implanted at valve 20, e.g., anchored to the septal leaflet. FIG. 31A represents the heart in ventricular diastole, and FIG. 31B represents the heart in ventricular systole. In some implementations, augmenter 300e comprises pouch 310. In some implementations, a stabilizer 362 comprises a tether extending from augmenter 300e.

In some implementations, a projection 360 of stabilizer comprises an anchor, e.g., a tissue anchor, configured to be anchored into tissue of the interventricular septum. In some implementations, and as shown, the tissue anchor can be configured to traverse the septal wall. In some implementations, stabilizer 362 is of a length such that the tether becomes tensioned during ventricular systole in a manner that limits atrial pivoting of pouch 310 during ventricular contraction.

In FIGS. 32A-B, augmenter 300f is shown implanted at valve 20, e.g., anchored to the septal leaflet. FIG. 32A represents the heart in ventricular diastole, and FIG. 32B represents the heart in ventricular systole. In some implementations, augmenter 300f comprises pouch 310.

In some implementations, a stabilizer comprises a projection 370 comprising a resilient spring. Projection 370, e.g., the resilient spring, can comprise a memory-shape alloy and/or can have a curved V-shape. The shape of projection 370 can be configured to cause projection 370 to rest and/or press against a wall of atrium 6 when augmenter 300f is anchored to a leaflet of valve 20, e.g., to the septal leaflet as shown. The spring provides a counterforce that resists pivoting of pouch 310 into atrium 6 during ventricular systole.

In FIGS. 33A-B, augmenter 300g is shown implanted at valve 20, e.g., anchored to the septal leaflet. FIG. 33A represents the heart in ventricular diastole, and FIG. 33B represents the heart in ventricular systole. In some implementations, augmenter 300g comprises pouch 310.

In some implementations, a stabilizer 382 comprises a tether extending from augmenter 300g and coupled to a bowed spring that defines a projection 380. Projection 380, e.g., the bowed spring, is shaped to extend circumferentially around the ventricular wall. The bowed spring is configured to press radially outward against the wall of the ventricle, providing a counterforce against a tendency of pouch 310 to pivot atrial-ward during ventricular systole.

In FIGS. 34A-B, augmenter 300h is shown implanted at valve 20, e.g., anchored to septal leaflet S, which is associated with chordae tendineae 18. FIG. 34A represents the heart in ventricular diastole, and FIG. 34B represents the heart in ventricular systole. In some implementations, augmenter 300h comprises pouch 310 and an anchor. In some implementations, augmenter 300h comprises a frame 330h to which pouch 310 and the anchor are coupled. In some implementations, a stabilizer 390 is coupled to pouch 310 and configured to be looped behind chordae tendineae 18 of leaflet S. In some implementations, stabilizer 390 is coupled to pouch 310 by being coupled to frame 330h.

In some implementations, stabilizer 390 comprises a rod (which can be tubular).

In some implementations, pouch 310 defines a transverse plane disposed between a proximal end of the pouch and the downstream opening, such that stabilizer 390 (e.g., the rod thereof) is configured to be positioned under leaflet S behind the chordae tendineae, and parallel to the transverse plane. In some implementations, the rod can extend laterally beyond the pouch parallel to the transverse plane.

In some implementations, stabilizer 390 comprises a wire 392 that is coupled to (e.g., threaded through) the rod. Wire 392 has a length that is adjustable in a manner that resists the upstream pivoting of the pouch. In some implementations, a clasp 394, e.g., a ring or a tube, can be coupled to frame 330h at the anchor in a manner that both ends of wire 392 can be threaded through clasp 394 to form an adjustable loop 396.

In some implementations, pulling on both ends of wire 392 enables adjustment of a circumference of loop 396 in a manner that draws stabilizer 390 closer to frame 330h. In some implementations, when a desired proximity of stabilizer 390 to frame 330h has been achieved, the ends of wire 392 are configured to be secured such that loop 396 maintains its length.

In the absence of the rod of stabilizer 390, tightening of the stabilizer (e.g., wire 392) may draw chordae tendineae 18 toward each other (e.g., bunching the chords together), which may deleteriously deform leaflet S in a manner that creates gaps between leaflet S and one or more of leaflets A and P. The rod of the stabilizer maintains the lateral width of the stabilizer even when the stabilizer is tightened, thereby advantageously reducing the likelihood of this deleterious effect.

In FIGS. 35A-D, augmenter 300i is shown implanted at valve 20, e.g., anchored to the septal leaflet. Each of FIGS. 35A-D represents the heart in ventricular diastole. In some implementations, augmenter 300i comprises pouch 310. In some implementations, a stabilizer 410 has a projection 400 comprising a stent, configured to be secured within a cardiovascular lumen by being expanded therein.

In FIG. 35A, stabilizer 410 comprises a resilient wire extending superiorly from augmenter 300i into the superior vena cava. In some implementations, projection 400 is configured to be expanded and secured within the superior vena cava, such that stabilizer 410 provides a counterforce to the hemodynamic pressure on valve 20 during ventricular systole, which would tend to cause augmenter 300i to pivot into atrium 6.

In FIGS. 35B-D, stabilizer 410 comprises a tether that couples projection 400, e.g., the stent, to augmenter 300i. In FIG. 35B, the cardiovascular lumen in which the stent is secured is the pulmonary artery. Tension on stabilizer 410 limits the ability of augmenter 300i to pivot into atrium 6 by pulling ventricular-ward from the downstream aspect of leaflet S. In FIG. 35C, the cardiovascular lumen in which the stent is secured is the inferior vena cava. Tension on stabilizer 410 limits the ability of augmenter 300i to pivot into atrium 6 by applying a counterforce to the pouch from an upstream surface of pouch 310 within the atrium. In FIG. 35D, the cardiovascular lumen in which the stent is secured is the coronary sinus.

In some implementations, similar to the function of stabilizer 410 in FIG. 35C, tension on stabilizer 410 limits the ability of augmenter 300i to pivot into atrium 6 by applying a counterforce to the pouch from an upper surface of pouch 310 within the atrium. In any of implementations of FIGS. 35B-D, the length of the tether can be adjusted intracorporeally in order to optimize the effect of the stabilizer 410.

In FIGS. 36, augmenter 300j is shown implanted at valve 20, e.g., anchored to the septal leaflet. FIG. 36 represents the heart in ventricular diastole. In some implementations, augmenter 300j comprises pouch 310. In some implementations, a stabilizer 454 defines a projection configured as a resilient wire (e.g., a bowed spring) extending from augmenter 300g.

In some implementations, the augmenter is configured to be anchored to a leaflet of valve 20, e.g., to the septal leaflet, such that the resilient wire presses against a wall of ventricle 8. In some implementations, the resilient wire provides a counterforce against a tendency of pouch 310 to pivot atrial-ward through valve 20 during ventricular systole.

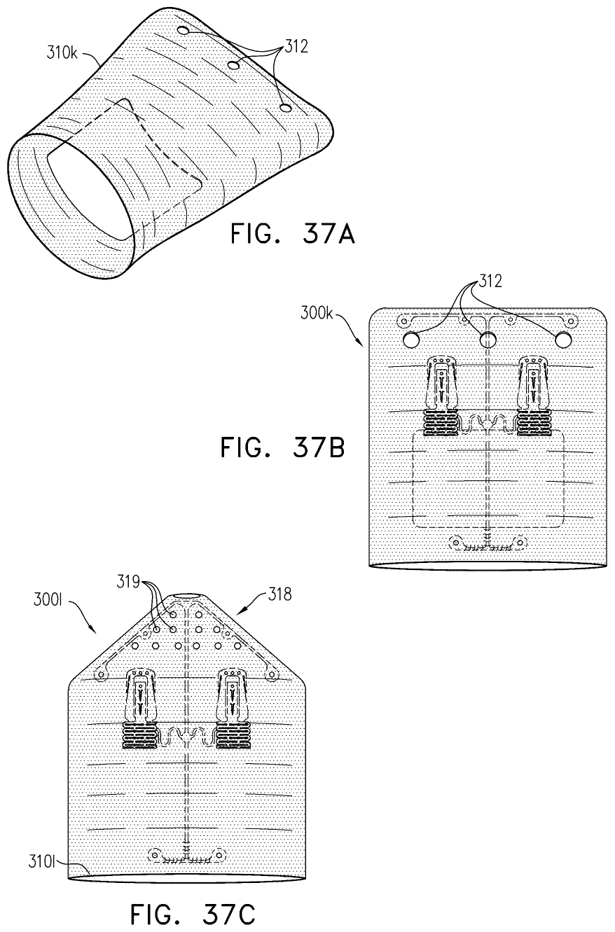

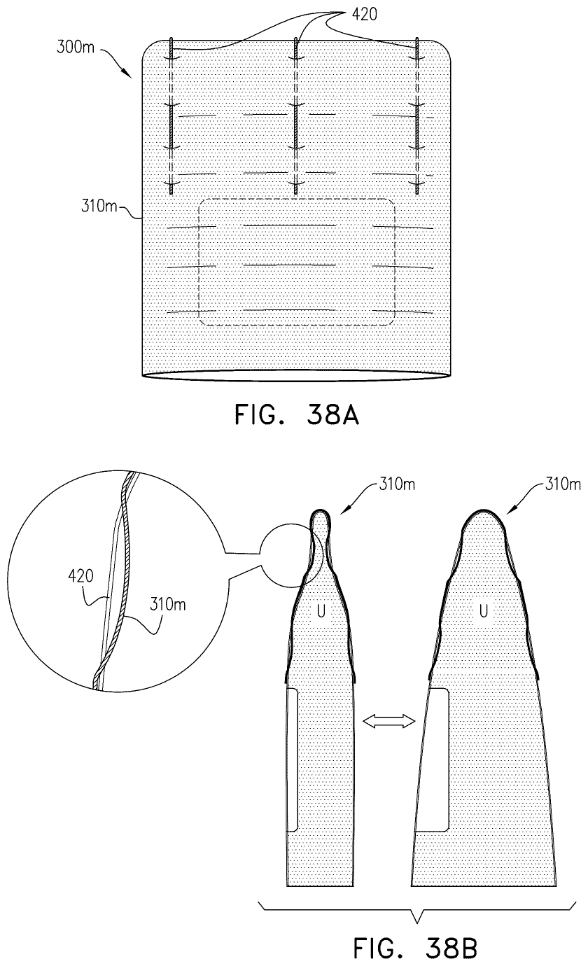

Reference is now made to FIGS. 37A-C and 38A-B. As described herein above, a leaflet augmenter (e.g., augmenter 300 and variants thereof, augmenter 60 and variants thereof) can comprise a compliant, flexible pouch, e.g., pouch 310 and/or pouch 262 and/or other variants described herein.

In some implementations, the pouch is configured to billow during ventricular systole to coapt with an opposing leaflet. During diastole, the pouch is configured to deflate and allow blood to circulate out. Thus, the pouch may hold more blood during systole than during diastole. The exchange of blood is important to prevent stagnation and thrombus formation. A deflation feature can be added to any of the pouches described herein to enhance exit and/or exchange of blood during the cardiac cycle. FIGS. 37A-C and 38A-B show examples of such features.

FIGS. 37A-C show an augmenter 300k, comprising a pouch 310k shaped to define one or more ports 312 at a proximal end of the pouch. In some implementations, ports 312 are disposed on a side of the pouch opposite the side to which the anchor is configured to anchor the augmenter to the leaflet. Ports 312 are configured to allow limited (e.g., controlled) retrograde blood flow therethrough into atrium 6 during billowing of pouch 310k during ventricular systole, e.g., due to increased ventricular pressure. In some implementations, ports 312 are provided with a one-way valve (not shown). In some implementations, the one-way valve can comprise, e.g., a cloth flap, e.g., a duck-bill valve, configured to open during diastole and close during systole. In some other implementations, the one-way valve can be configured to open during systole and close during diastole. The valve can be created by, e.g., making a laser cut in pouch 310k and/or by suturing a flap thereto.

FIG. 37C shows augmenter 300l having an alternate shape of pouch 310. In some implementations, corners 316 of pouch 310k, e.g., as shown in FIGS. 37A-B, may be rounded to create smooth shoulders 318, e.g., as shown in pouch 310l. Such rounded shoulders may advantageously facilitate exit of blood from pouch 310 during diastole. In some implementations, and as shown, pouch 310l may have perforations 319 to further facilitate drainage of blood.

FIGS. 38A-B show an augmenter 300m, comprising a pouch 310m. In some implementations, augmenter 300m comprises one or more springs 420, e.g., resilient wires, coupled to an upstream part U of pouch 310m. Springs 420 can comprise, e.g., a memory-shape alloy.

In some implementations, springs 420 are braided or sutured into pouch 310m. In some implementations, springs 420 are configured to facilitate emptying of pouch 310m (e.g., upstream part U thereof) during diastole by squeezing blood out of upstream part U during ventricular diastole. That is, the spring constant of springs 420 is such that (i) during ventricular systole, ventricular pressure can overcome (e.g., strain) the springs to billow upstream part U, and (ii) during ventricular diastole, the springs relax and squeeze the blood out of the upstream part of the pouch.

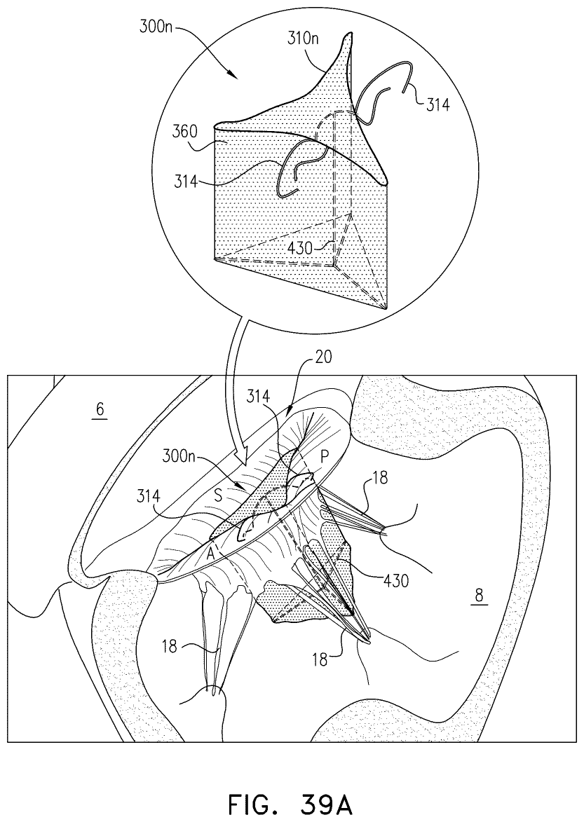

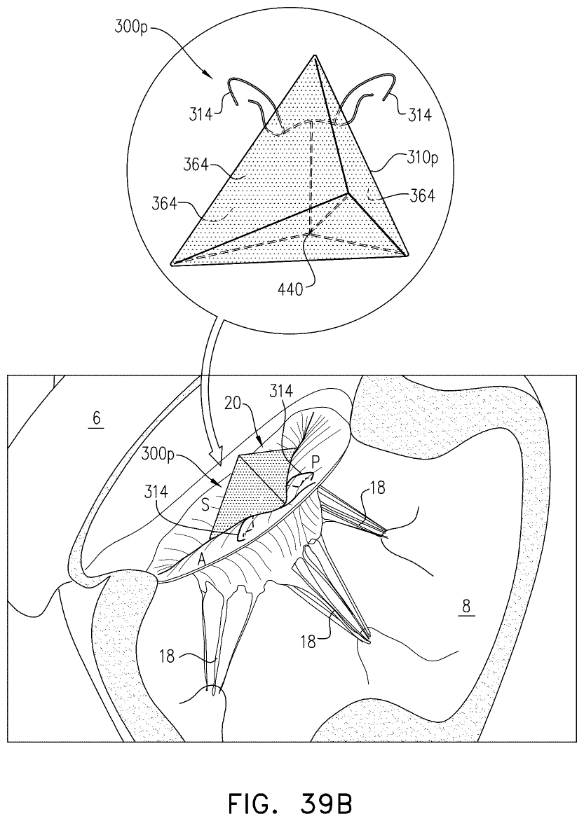

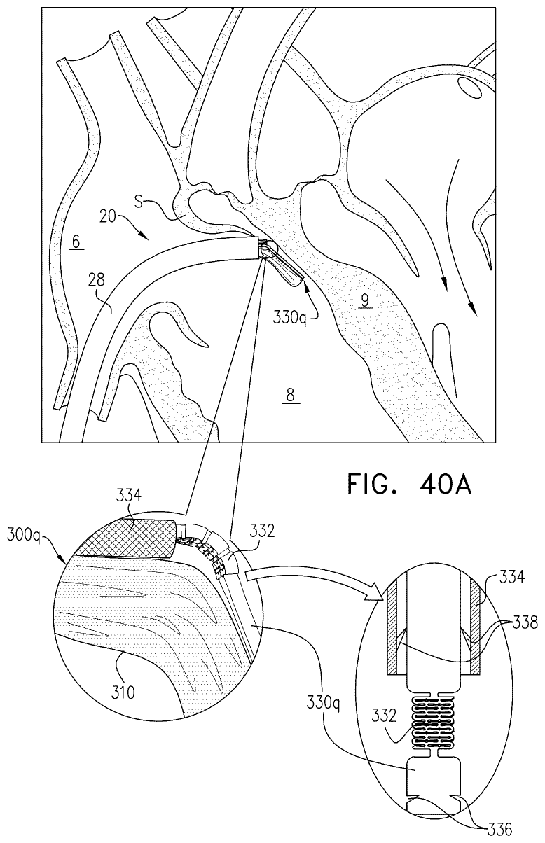

Reference is now made to FIGS. 39A-B, showing augmenter 300n, according to some implementations. In some implementations, augmenter 300n is shown implanted at valve 20, anchored to leaflets A and P. As mentioned, the tricuspid valve leaflets have greater compliance than those of the mitral valve. Thus, for an augmenter anchored to a single leaflet of the tricuspid valve, the opposing leaflets may provide an opening through which an implant, e.g., leaflet augmenter, may flap. By configuring a leaflet augmenter with a triangular or pyramidal shape, and anchoring the pouch to two leaflets, the augmenter is provided with enhanced stabilization, as well as enhanced sealing against the leaflets of the tricuspid valve. In some such implementations, an unanchored face of the pouch lies parallel to the septal leaflet.

As shown in FIGS. 39A, augmenter 300n comprises a pouch 310n and two anchors 314. Each anchor 314 is coupled to a respective one of the three triangular faces or external surfaces of pouch 310n. In some implementations, augmenter 300n comprises an internal frame 430, to which anchors 314 are fixedly coupled. In some implementations, frame 430 comprises a mast structure with an upstream aspect coupled to anchors 314, and one or more lateral extensions extending from a downstream aspect.

In some implementations, the lateral extensions can be sutured or otherwise attached at a downstream opening of pouch 310n. Anchors 314 are configured to anchor the first and second faces, respectively, to leaflets A and P.

In some implementations, a third face or external surface is configured to face leaflet S. During ventricular systole, the external surface of the third face of the pouch coapts against leaflet S.

In some implementations, pouch 310n is dimensioned such that, during ventricular systole, pouch 310n projects into ventricle 8.

In FIG. 39B, augmenter 300p comprises a pouch 310p with a pyramidal shape, e.g., having three faces 364 and a downstream opening. In some implementations, frame 440 comprises a mast structure with an upstream aspect coupled to anchors 314, and three lateral extensions extending from a downstream aspect of the mast structure to form a triangle. In some implementations, the lateral extensions can be sutured or otherwise attached at the downstream opening of pouch 310p to form a pyramidal structure of augmenter 300p. In some implementations, pouch 310p is dimensioned such that, during ventricular systole, pouch 310p projects into atrium 6.

Reference is made to FIGS. 40A-B, showing an augmenter 300q, according to some implementations. During a delivery procedure to anchor the augmenter to the leaflet, a tilt of the delivery tool may be needed to slip the anchor under the leaflet. Tilting the augmenter in such a manner may cause a downstream end of the frame to encounter the septal wall, especially in cases of ventricular hypertrophy. To enable such tilting and anchoring, and/or to prevent possible injury to the septal wall, a downstream segment of the frame may have a flexibility during the procedure that is eliminated toward the end of the procedure, e.g., a rigidity required for the frame to serve its purpose after implantation may be conferred upon it only after anchoring of the augmenter to the leaflet. This increased flexibility may also be useful during transluminal delivery of the augmenter, e.g., allowing the augmenter to navigate tight bends.

In some implementations, augmenter 300q comprises a pouch 310 and a frame 330q. Frame 330q comprises a hinge 332 that confers flexibility on a segment of frame 330q at which hinge 332 is disposed. Frame 330q further comprises a locking tube 334 slidably coupled to hinge 332, such that sliding tube 334 over hinge 332 stiffens the segment. Tube 334 can be provided with teeth 338 that are configured to slide into notches 336 of frame 330q and lock tube 334 in place over hinge 332.

As shown in FIG. 40A, augmenter 300q is in the process of being delivered and anchored by delivery tool 28 to leaflet S of valve 20. As shown in FIG. 40B, after anchoring to leaflet S, tube 334 has been slid over hinge 332, thus stiffening the downstream segment of frame 330q. In some implementations, locking tube 334 is actuated (e.g., pushed down) by an actuation rod of delivery tool 28.

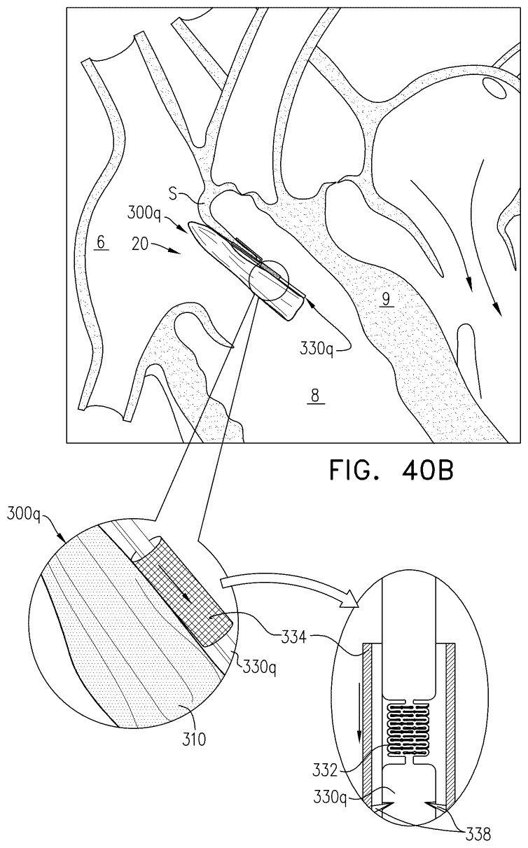

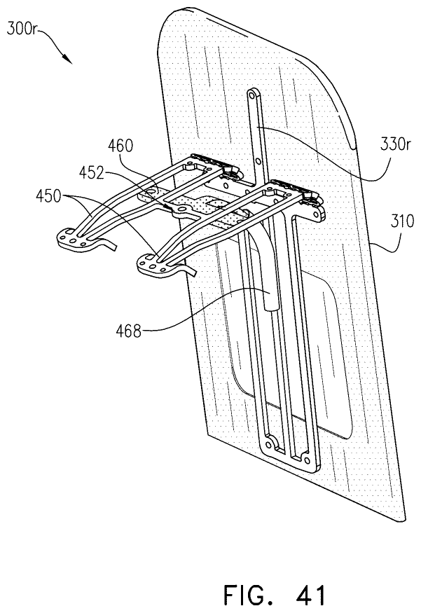

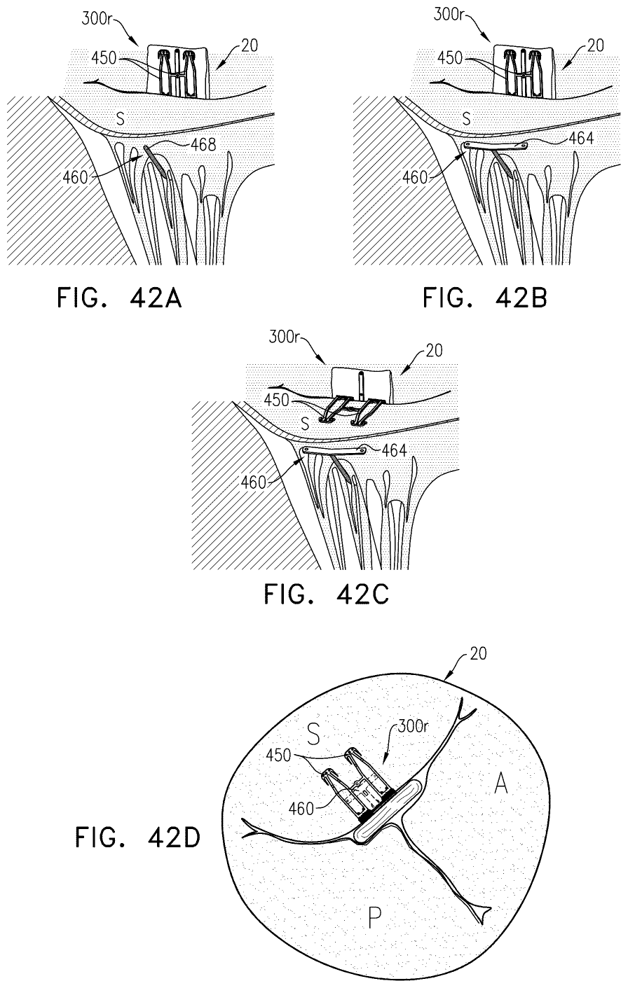

Reference is made to FIGS. 41, 42A-D, and 43A-C, showing augmenter 300r according to some implementations. In some implementations, including several described hereinabove, the anchor of the augmenter is a clasp that has an upstream clasp element and a downstream clasp element, hingedly coupled to the frame of the augmenter, and configured to anchor the augmenter to the leaflet by clasping the leaflet between these clasp elements. It may be desirable to provide a widened anchor (e.g., a clasp) in order to grasp a larger area of the leaflet. However, a wider downstream clasp element may more easily become obstructed by, and/or may inadvertently clasp, the chordae tendineae of the leaflet to which the augmenter is being anchored. In order to address this, the downstream clasp element may be laterally expandable after it has been passed between the chordae tendineae and is disposed at the downstream surface of the leaflet, as illustrated and described below. The added surface area acts as a pressure reduction feature by spreading the pressure out on the downstream leaflet surface.

FIG. 41 shows augmenter 300r according to some implementations. In some implementations, augmenter 300r comprises a pouch 310 and a frame 330r. In some implementations, the anchor comprises a pair of upstream clasp elements 450 connected by a lateral bridge 452, and a downstream clasp element 460. In some implementations, a tube 468 is slidably coupled to downstream clasp element 460. In some implementations, tube 468 can restrain lateral elements of downstream clasp element 460 prior to deployment, as further described with respect to FIGS. 43A-C herein below.

FIGS. 42A-D illustrate steps in a procedure to anchor augmenter 300r to leaflet S. In FIG. 42A, a delivery tool (not shown) has transluminally advanced augmenter 300r to valve 20 and positioned downstream clasp element 460 at a downstream side of leaflet S by passing the downstream clasp element between chordae tendineae of the leaflet. In FIG. 42B, tube 468 has actuated downstream clasp element 460 to expand laterally. In FIG. 42C, augmenter 300r has been anchored to leaflet S by clasping leaflet S between the laterally-expanded downstream clasp element 460 against the downstream side of leaflet S, and the upstream clasp element 450 against an upstream side of leaflet S. FIG. 42D is a top-down view of the same state shown in FIG. 42C.

FIGS. 43A-C show respective variants of downstream clasp element 460, in accordance with some implementations.

FIG. 43A shows a downstream clasp element 460a that the delivery tool is configured to actuate by retracting a tube 468a and/or tensioning a line 462a, such that the downstream clasp element expands outward to form lateral wings 464a. In some implementations the lateral expansion occurs responsively to the retraction of tube 468a. In some implementations, tensioning line 462a stabilizes wings 464a in their lateral configuration. In some implementations, the lateral expansion occurs responsively to tensioning of line 462a.

FIG. 43B shows a downstream clasp element 460b that is actuated by being pushed out of a tube 468b. For example, a spring 467 can be maintained in a stressed state by a wire 462b such that, upon release of the wire, clasp element 460b springs forward. In some implementations, hinges 463 pivot wings 464b laterally outward responsively to exposure of the wings from the tube. In some implementations, wings 464b are curved such that when they encounter a tissue surface such as the septal wall, they are pushed apart to deflect laterally outward.

FIG. 43C shows a downstream clasp element 460c, in which a pin 465 restrains arms 464c close to the main structure of downstream clasp element 460. Upon release of pin 465 by pulling on wire 462c, arms 464c are released and spring laterally outward.

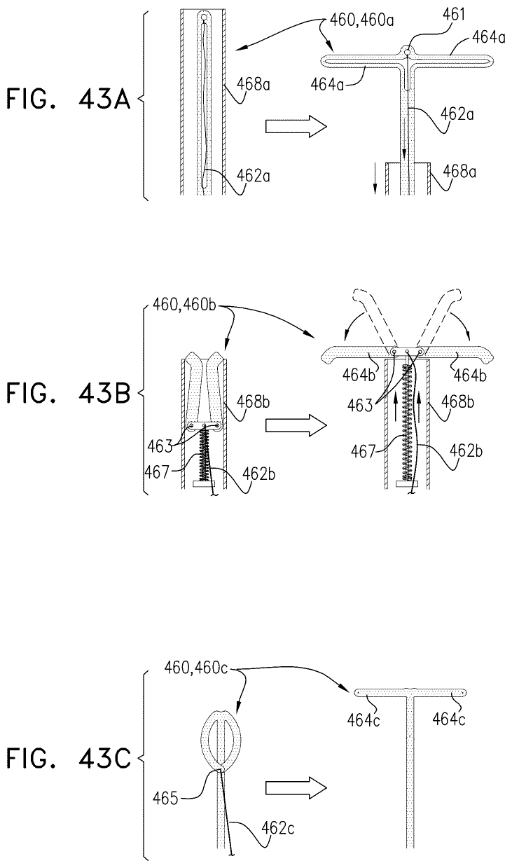

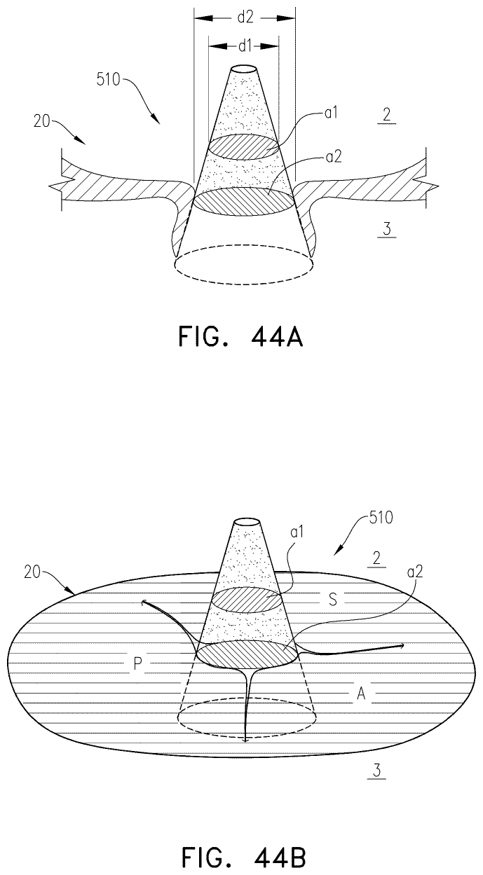

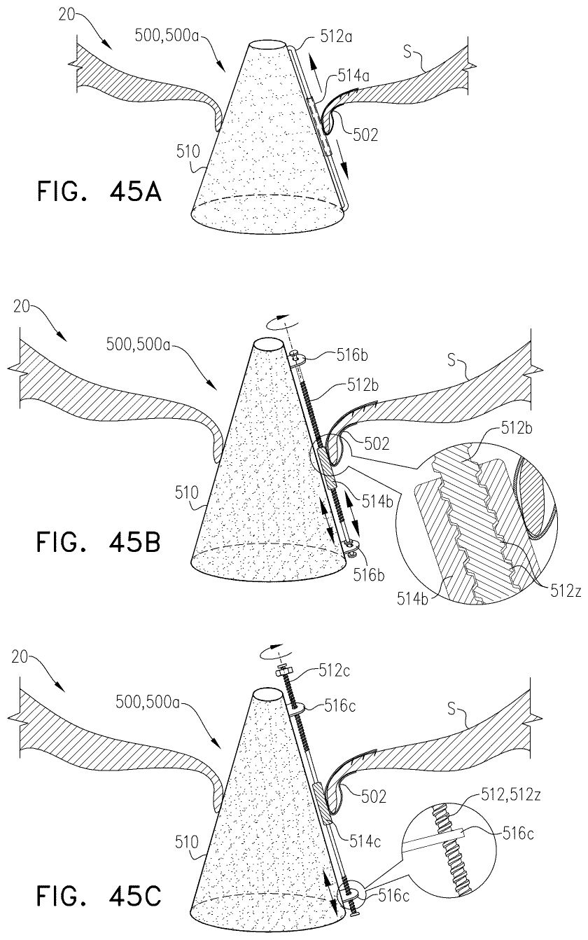

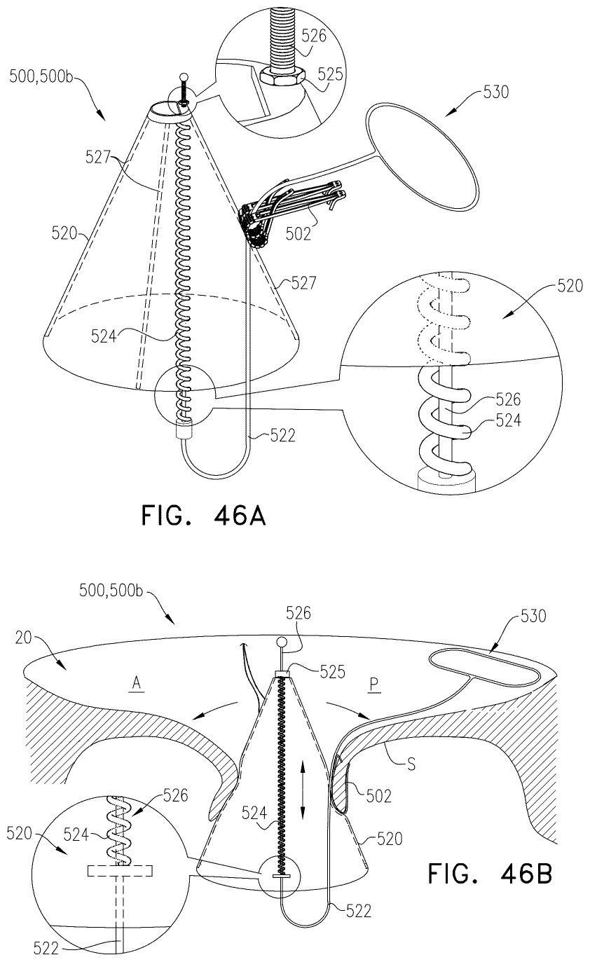

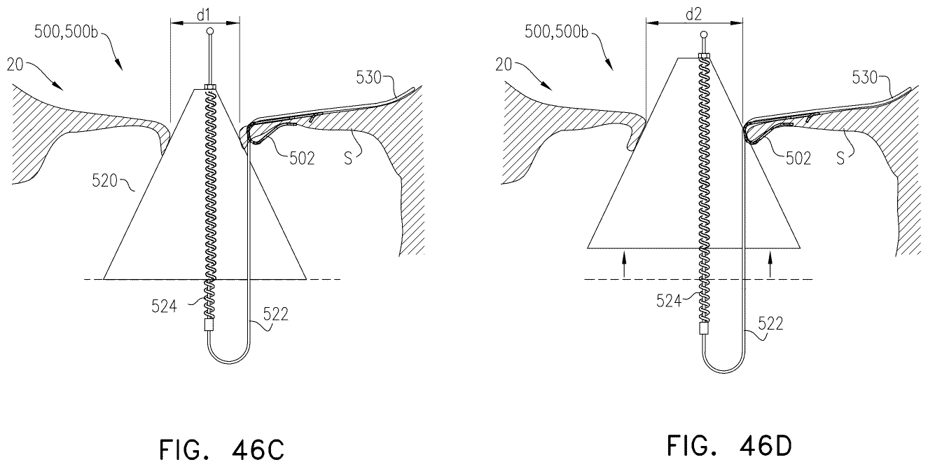

Reference is now made to FIGS. 44A-51, which are schematic illustrations of a leaflet augmenter 500 (e.g., variants thereof), implanted at a valve 20 of a heart 4, in accordance with some implementations. Augmenter 500 can share features of any of augmenters 30, 50, 60 and 300, described herein above. Similarly, any of augmenters 30, 50, 60, and 300 can be modified to include features of augmenter 500 (e.g., of any variant thereof), such as its stabilization features. Illustrated in these figures, valve 20 is a valve with three leaflets, e.g., the tricuspid valve, disposed between the right atrium 2 and the right ventricle 3 of heart 4. Where labeled, the leaflets are designated as S for septal, P for posterior, and A for anterior, respectively. In some implementations, augmenter 500 (e.g., variants thereof) can be used at a bicuspid valve such as the mitral valve, mutatis mutandis. Augmenter 500 variant 500a is shown in FIGS. 45A-C, and variants 500b-500h are shown in FIGS. 46A-51.

In some implementations, augmenter 500 (e.g., variants thereof) comprises a conical pouch 510. The cone of pouch 510 can define a vent, e.g., one or more ports, at an apical region, the vent comprising an opening into atrium 2. The cone of pouch 510 can further define a base forming a downstream opening of the pouch, positioned to face (e.g., open into) ventricle 3. Pouch 510 can comprise a flexible fabric that enables the patch to inflate during systole, coapting against both the leaflet to which the augmenter is anchored and one or more of the other (e.g., opposing) leaflets, thus inhibiting regurgitation during ventricular systole. Although the figures show augmenter 500 anchored to septal leaflet S, the augmenter can be anchored to one of the other leaflets. In some implementations, augmenter 500 can be tailored toward anchoring to a particular one of the leaflets.

The pouch can be configured in a way that its position with respect to the atrium and ventricle (e.g., its position along an atrioventricular axis) is adjustable to suit anatomical features of valve 20, thus improving coaptation of pouch 510 with one or both opposing leaflets A and P.

Pouch 510 can comprise one or more, e.g., 2, 3, or 4, reinforcing wires 527. Wires 527 can have a stiffness greater than that of the fabric of pouch 510, and are configured to provide stability to the pouch by deflecting outward as the pouch inflates. Reinforcing wires 527 can be configured to inhibit the fabric of the pouch from flapping atrial-ward in response to hemodynamic forces from ventricular contraction. While shown most clearly in association with pouch 510 of augmenter 500b in FIG. 46A, reinforcing wires 527 can be included with any variant of augmenter 500, and mutatis mutandis, with any of the augmenters described herein above.

In FIG. 44A-B, conically-shaped pouch 510 (e.g., of augmenter 500) is shown positioned in a gap between the three leaflets of valve 20. For clarity, other components of the augmenter are not shown in FIGS. 44A-B. FIG. 44A shows the valve in cross-section and FIG. 44B shows the valve in perspective view.

In some implementations, and as described in more detail hereinbelow, the augmenter comprises an adjustment mechanism configured to facilitate adjustment of positioning between the anchor and the pouch while the pouch remains anchored to the lip by the anchor, in a manner that changes a proportion of the pouch that is disposed within the atrium during ventricular systole. Adjusting this position enables wider or narrower regions of the pouch to be disposed between the leaflets during ventricular systole, thereby allowing the effective width of the pouch to be adjusted intraprocedurally in order to achieve optimal coaptation. For example, for a given valve and a given anchoring site, a wider region of the pouch having a larger cross-sectional area a2 and/or a greater width d2 may be optimal, whereas for a different valve and/or anchoring site, a narrower region of the pouch having a smaller cross-sectional a1 and/or a smaller width d1 may be optimal.

In some implementations, augmenter 500 comprises an anchor 502 configured to anchor augmenter 500 to leaflet S. Anchor 502 can be any of the anchors described herein above. In some implementations, anchor 502 can be a clasp that is configured to anchor the augmenter to the leaflet by clasping the leaflet between these clasp elements. In some implementations, the anchor is coupled to the pouch in a manner that facilitates adjusting a position of pouch 510 with respect to the atrium. In some implementations, the augmenter comprises a stem, configured to couple the anchor to the pouch and to facilitate adjusting a position of the pouch with respect to the atrium.

Reference is made to FIGS. 45A-C, showing an augmenter 500a according to some implementations. In some implementations, pouch 510 is configured to be coupled to anchor 502 via an adjustable stem 512 (e.g., variants 512a, 512b, or 512c). Stem 512 can be disposed through sleeve a 514 (e.g., variants 514a, 514b, or 514c). Sleeve 514 can be fixed to anchor 502 in a manner that fixedly, slidably, or rotatably couples stem 512 to anchor 502. In some implementations, stem 512 can be fixedly or rotatably coupled to pouch 510. Without limiting the range of possible couplings, specific examples of such couplings are described with respect to FIGS. 45A-C.

The coupling of stem 512 to pouch 510 and to anchor 502 is configured to enable adjustment of a position of cone-shaped pouch 510. For example, when anchor 502 is anchored to leaflet S, pouch 510 can be moved either in the direction of atrium 6 or in the direction of ventricle 8. Adjusting the position of the pouch can be accomplished in a variety of ways, as described herein below.

In FIG. 45A, stem 512a comprises a rod that is rotatably coupled, e.g., via a rotary bearing at either end of stem 512a, to both the apical region and the downstream region of pouch 510, e.g., along an external surface of the pouch. A midsection of stem 512a is slidably coupled to anchor 502 via sleeve 514a.

When augmenter 500a is anchored to leaflet S, sliding stem 512a along sleeve 514a adjusts a position of pouch 510 either atrial-ward or ventricular-ward. Sliding pouch 510 in an atrial direction positions wider regions of the pouch between the first leaflet and the opposing leaflet(s), thereby providing a greater surface area for coaptation. Sliding pouch 510 in a ventricular direction positions narrower regions of the pouch between the first leaflet and the opposing leaflet(s), thereby providing a lesser surface area for coaptation.

In FIG. 45B, stem 512b is rotatably coupled to sleeve 514b, e.g., via a rotary bearing such as eyelets 516b. Eyelets 516b are disposed, e.g., at apical and downstream regions, respectively, of pouch 510. The rotatable coupling enables stem 512b to rotate but not slide with respect to the eyelets. Stem 512b has external threads 512z along a midsection thereof. Sleeve 514b has internal threads, such that stem 512b is threadably rotatable through sleeve 514b.

When augmenter 500a is anchored to leaflet S, rotating stem 512b along sleeve 514b adjusts a position of both stem 512b and pouch 510. Rotating stem 512b in a first direction moves both stem 512b and pouch 510 toward the atrium, and rotating stem 512b in an opposite direction moves both stem 512b and pouch 510 toward the ventricle.

In FIG. 45C, stem 512c is coupled to anchor 502 by being disposed through sleeve 514c in a manner that enables stem 512c to rotate but not slide with respect to sleeve 514c, e.g., via a rotary bearing. Stem 512c is coupled to pouch 510 via a threaded nut, e.g., via eyelets 516c, shown disposed at the apical and downstream regions of pouch 510, respectively. Stem 512c moves through eyelets 516c by external threads 512z that rotate along internal threads of the eyelets.

When augmenter 500a is anchored to leaflet S, rotating stem 512c within eyelets 516c moves pouch 510 along stem 512c. Rotating stem 512c in a first direction moves pouch 510 toward the atrium, and rotating stem 512c in an opposite direction moves pouch 510 toward the ventricle.

Comparing the example implementations shown in FIGS. 45B-C, in FIG. 45C, only the pouch moves atrial-ward or ventricular-ward along stem 512c, whereas in FIG. 45B both pouch and stem move with respect to sleeve 514b. In both example implementations, the sleeve is fixed with respect to anchor 502. As mentioned, the illustrated implementations are example solutions and not intended to be limited to those shown in FIGS. 45A-C.

Reference is made to FIGS. 46A-D, showing an augmenter 500b according to some implementations. Augmenter 500b comprises a conical pouch 520 configured to be coupled to anchor 502 via an adjustable stem 522. Variants of stem 522 (5220, 522d, 522e, 522f, 522g, and 522h) are shown in FIGS. 47A-50.

Stem 522 is configured to couple pouch 520 to anchor 502 by extending from the anchor toward the downstream opening, outside of the pouch. Under a rim of the pouch at the downstream opening, the stem defines a curve or bight of about 180 degrees, such that the stem makes a U-turn to extend inside the pouch toward the apical region. The portion of stem 522 disposed within pouch 520 defines a mast 526 coupled to the apical region, e.g., via a threaded nut 525. The distal part of mast 526 can be threaded to match the threading of nut 525. The U-shaped stem advantageously provides direct coaptation of the pouch with the opposing leaflet(s) of the valve.

Augmenter 500b can comprise an adjustment spring 524 configured to be disposed coaxially around mast 526 of stem 522 on the interior of pouch 520. Spring 524 serves the purpose of holding pouch 520 aloft with respect to mast 526. Rotating nut 525 in a first direction compresses the spring, e.g., places spring 524 under strain. Rotating nut 525 in an opposite rotational direction is configured to relax spring 524.

When augmenter 500b is anchored to leaflet S, the U-shape of stem 522 advantageously allows pouch 520 to shift laterally in a plane of valve 20. The flexibility provided by stem 522 allows pouch 520 to adjust itself to a position that minimizes a gap between the first leaflet and the opposing leaflet during hemodynamic forces created during ventricular contraction.

As shown in FIGS. 46C-D, when augmenter 500b is anchored to leaflet S, rotating nut 525 in a first rotational direction, e.g., such that a portion of mast 526 outside of pouch 520 increases, is configured to move pouch 520 in a first direction, e.g., toward the ventricle. Adjustment of pouch 520 in a ventricular-ward direction decreases a diameter d1 of pouch 520 available to coopt with the leaflets of valve 20 (FIG. 46C). Rotating nut 525 in an opposite direction, e.g., such that a portion of mast 526 inside of pouch 520 increases, is configured to move pouch 520 in a second direction, e.g., toward the atrium. Adjustment of pouch 520 in an atrial-ward direction increases a diameter d2 of pouch 520 available to coapt with the leaflets of valve 20 (FIG. 46D).

Reference is made to FIGS. 47A-B, showing representative augmenter 500c according to some implementations. Augmenter 500c comprises a conical pouch 520 configured to be coupled to anchor 502 via adjustable stem 522c. Stem 522c defines a mast 526′ extending through the interior to the apical region. Mast 526 and the apical region of pouch 520 comprise a ratchet mechanism. In some implementations, and as shown, the ratchet mechanism can comprise, e.g., teeth 528 on mast 526′ interacting with at least one pawl (not shown) at the apical region. In some implementations, the ratchet mechanism can comprise teeth on the apical region interacting with one or more pawls on mast 526′.

When augmenter 500c is anchored to leaflet S, adjustment of a position of pouch 520 with respect to anchor 502 can be accomplished by ratcheting mast 526′ through the apical region. The ratcheting mechanism can be configured to adjust a position of pouch 520 with respect to anchor 502. The selected direction of adjustment, e.g., the direction of ratcheting, can be configured either atrial-ward or ventricular-ward. As shown in FIG. 47A, pouch 520 can initially be positioned with a larger cross-sectional area a2 disposed at valve 20. As mast 526′ is ratcheted in an atrial direction, pouch 520 is adjusted in a ventricular direction, such that a smaller cross-sectional area a1 remains disposed between the leaflets of valve 20, as shown in FIG. 47B. In some implementations, ratcheting can be accomplished by pulling mast 526′ through the apical region via, e.g., a delivery tool 504 comprising a pull wire 506.

Reference is made to FIGS. 48A-B, showing representative augmenter 500d according to some implementations. Augmenter 500d comprises a conical pouch 520 and a sleeve 522d′, fixed to anchor 502. Pouch 520 is coupled to anchor 502 via adjustable stem 522d. Stem 522d comprises a first region that is slidable through the sleeve, and a second region defining a mast 522d″. Mast 522d″ is configured to be fixed to the apical region of pouch 520, and can have a stiffness greater than that of the first region. A threaded nut 529 at the atrial aspect of sleeve 522d′ is configured such that rotating nut 529 adjusts a position of stem 522d along sleeve 522d′.

When augmenter 500b is anchored to leaflet S, sliding stem 522d through sleeve 522d′ adjusts a position of pouch 520 with respect to anchor 502 by progressively sliding the first region through the sleeve. Adjustment of pouch 520 in a ventricle-ward direction by sliding the stem through the sleeve in an atrial-ward direction positions progressively narrower regions of the pouch between the first leaflet and the opposing leaflet (FIG. 48A). Conversely, adjustment of pouch 520 in an atrial-ward direction by sliding the stem through the sleeve in a ventricular direction disposes progressively wider regions of the pouch between the first leaflet and the opposing leaflet (FIG. 48B).

Reference is made to FIGS. 49A-C, showing representative augmenter variants 500e-g according to some implementations. Each of augmenters 500e, 500f, and 500g comprises a pouch 520 coupled to an anchor 502 via a stem 522 (e.g., variants 522e, 522f, 522g). In some implementations, and as shown, stem 522 comprises a first part that extends from anchor 502 toward the downstream opening, and a second part that defines a mast that extends through the interior to the apical region. The mast can be coupled to pouch 520 at the apical region thereof. The first part and the second part of stem 522 are coupled via an adaptive joint 523 (e.g., variants 523e, 523f, 523g). The adaptive joint 523 can comprise a variety of self-adjusting mechanisms.

FIG. 49A shows an adaptive joint comprising a pivot joint 523e near a vertex of stem 522e. The pivot joint provides a single degree of freedom of the stem. A spring 521 is configured to span the first part and the second part around the joint. A stiffness of spring 521 can be selected to provide a desired amount of flexibility to the joint.

FIG. 49B shows an adaptive joint comprising a ball and socket joint 523f at a vertex of stem 522f. A ball and socket joint provides more degrees of freedom of movement on stem 522f.

FIG. 49C shows an adaptive joint comprising an X-Y mount 523g that provides two degrees of freedom in a lateral plane between parts 522g′ and 522g″.

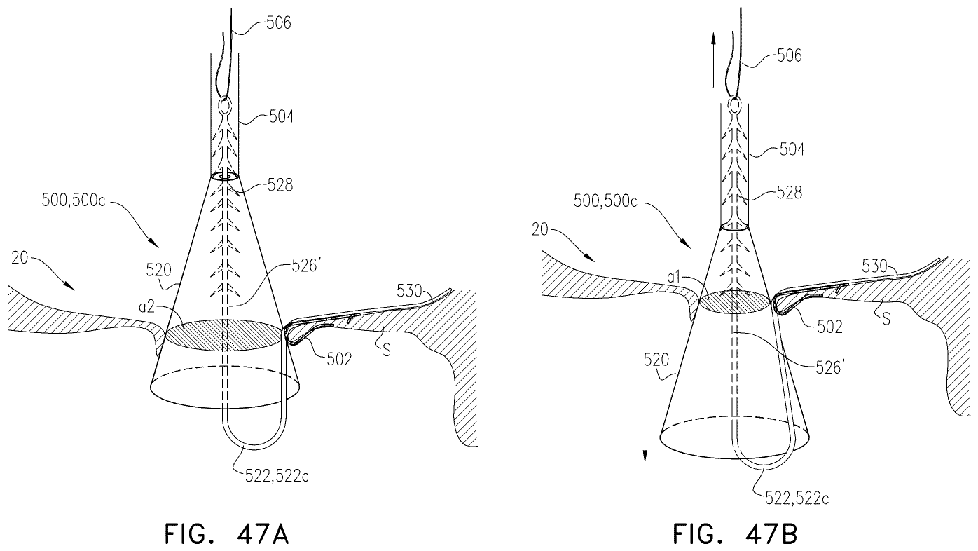

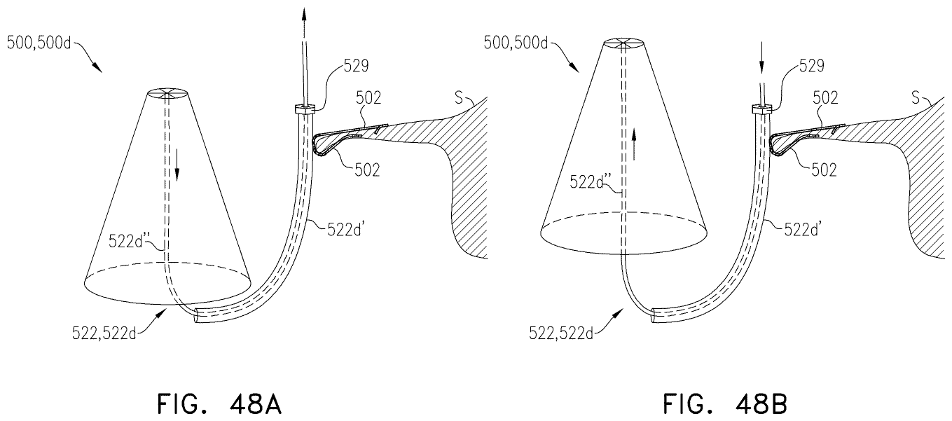

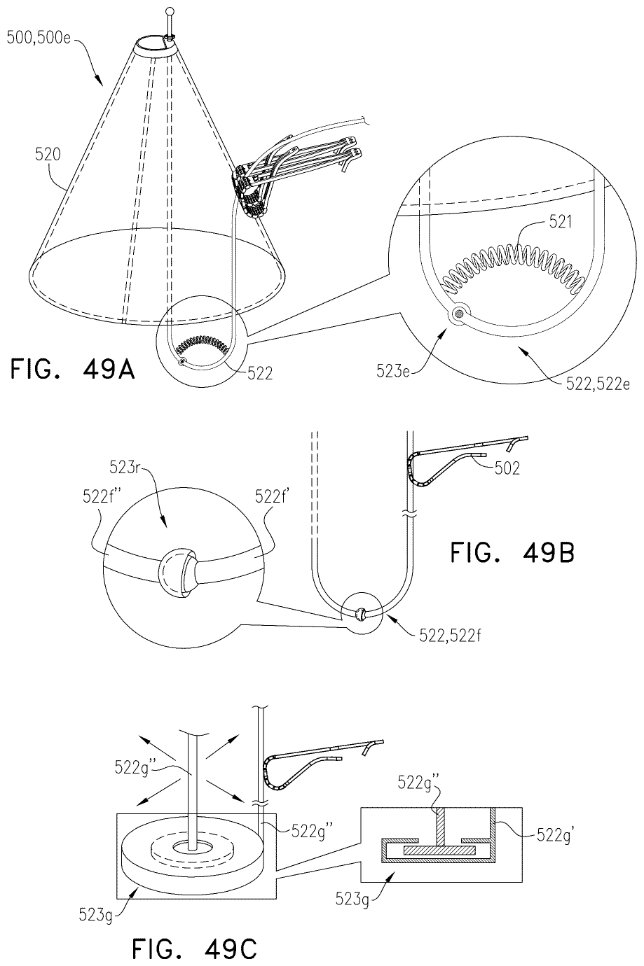

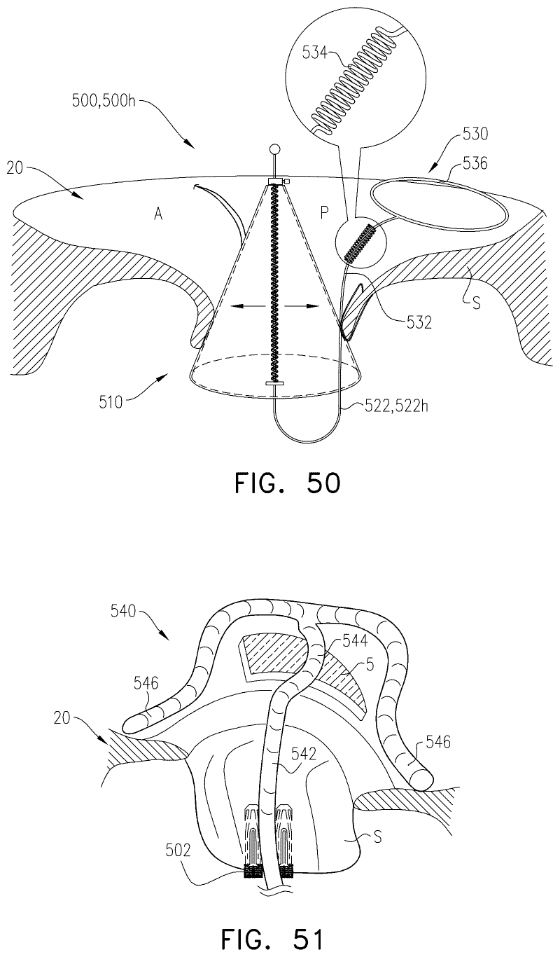

Reference is made to FIGS. 50 and 51, showing a representative augmenter 500h anchored to leaflet S of a valve, further comprising an example stabilizer (530, 540). Stabilizers 530, 540 are configured to advantageously stabilize variants of augmenter 500 by inhibiting excessive movement of pouch 510 due to hemodynamic forces during ventricular contraction. While stabilizers 530, 540 are shown coupled to augmenter 500h, the stabilization features provided by these stabilizers can be used with any of the leaflet augmenters described herein above.

In FIG. 50, augmenter 500h is shown anchored to the lip of leaflet S. In some implementations, and as shown, a stabilizer spring 534 is disposed along rod 532. In various subjects, a lip-to-annulus distance of the septal leaflet may vary. Spring 534 is configured to provide flexibility to a length of rod 532, and thus to a position of loop 536, with respect to the annulus of valve 20. The flexibility in position of loop 536 enables stabilizer 530 to be suitable for valves of various sizes. In some implementations, stabilizer 530 can comprise a rod 532 and a loop 536 without a stabilizer spring, e.g., as shown in FIGS. 46A-B.

Stabilizer 530 is configured to extend from the augmenter, e.g., from anchor 502, across leaflet S, and to rest on an annulus of valve 20, as similarly shown for augmenter 500b in FIG. 46B. When augmenter 500h is anchored to the lip of leaflet S, rod 532 lies across leaflet S, and loop 536 rests on an annulus of valve 20.

In FIG. 51, anchor 502 is shown attached to the lip of leaflet S. For clarity, the remainder of augmenter 500h is not shown. Stabilizer 540 comprises a pair of curved arms 546. Curved arms 546 are configured to extend laterally in opposite directions from a stem 542 extending from the augmenter, e.g., from anchor 502, over septal leaflet S. Stem 542 comprises an arc 544 configured to form an elevation above the anchoring of the anchor to the septal leaflet, e.g., over a triangle of Koch 5. Arc 544 is configured to avoid contact with the tissue, thus advantageously decreasing a risk of irritation by juxtaposition with electrically excitable cardiac tissue in the region. Arms 546 are configured to rest above the annulus, e.g., on the septal wall to either side of the triangle of Koch, or above an atrioventricular node of the heart.

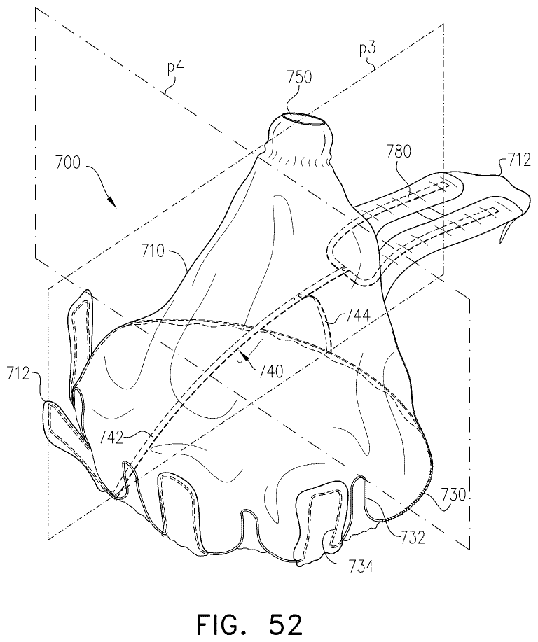

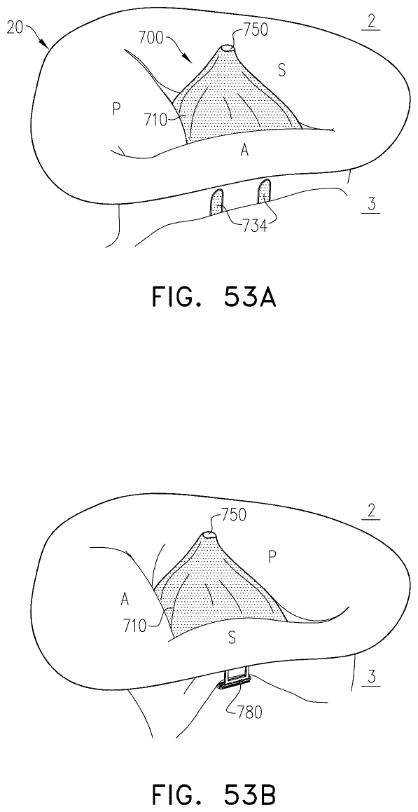

Reference is made to FIGS. 52 and 53A-B, which are schematic illustrations of a leaflet augmenter 700, in accordance with some implementations. FIG. 52 shows augmenter 700, and FIGS. 53A-B show augmenter 700 implanted at a valve 20 of a heart 4, in accordance with some implementations. Illustrated in these figures, valve 20 is a valve with three leaflets, e.g., the tricuspid valve, disposed between the right atrium 2 and the right ventricle 3 of heart 4. Where labeled, the leaflets are designated as S for septal, P for posterior, and A for anterior, respectively. In some implementations, augmenter 700 (e.g., variants thereof) can be used at a bicuspid valve such as the mitral valve, mutatis mutandis.

Augmenter 700 comprises a pouch 710 comprising a flexible material. In some implementations, and as shown, pouch 710 may have a conical shape. In some implementations, pouch 710 may comprise a vent 750 at the upstream end of the conical shape comprising an opening into atrium 2. The vent is configured to allow a limited amount of blood to flow retrogradely into the atrium during ventricular systole. Pouch 710 may be shaped to hemodynamically facilitate, e.g., to funnel, blood through the vent, to facilitate maximal flushing of the pouch. In some implementations, vent 750 may be a simple opening in pouch 710. In some implementations, vent 750 may have a component that provides structure and/or function, such as a grommet. The size and shape of vent 750 may be configured to prevent hemodynamic flow conditions that could otherwise cause hemolysis of red blood cells.

In some implementations, the vent comprises a check valve, such as a flap, a reed valve, a diaphragm valve, a duckbill valve, and the like. Irrespective of whether a valve element is included, vent 750 is shaped and sized to allow passage of a specific volume of blood—e.g., to facilitate optimal (e.g., complete) flushing of pouch 710 during each cardiac cycle (e.g., during each ventricular systole) without promoting excessive regurgitation into the atrium. Such flushing may advantageously prevent stagnation and/or thrombosis of blood within the pouch.

Reference is again made to FIGS. 52-53B, which illustrate one or more legs 734 protruding externally from pouch 710, e.g., away from the downstream opening and/or toward the opposing leaflet. Each leg 734 may be shaped and/or positioned such that, as the external surface of the augmenter coapts against the opposing leaflet (e.g., during ventricular systole), the leg tucks under the opposing leaflet—or, in the case of an augmenter for the tricuspid valve, under one or more of the opposing leaflets.

Legs 734 may be arranged (e.g., distributed) circumferentially around the downstream opening. In some implementations, the legs may be attached to a frame 730 at the downstream opening of the pouch. In some implementations, the legs may vary in size. In some implementations, the legs may have a rectangular shape, triangular shape, elliptical shape, and/or an irregular shape (e.g., having a bulbous shape at a distal end of the leg).

Frame 730 may be coupled to pouch 710 along at least part of a perimeter of the downstream opening of the pouch—e.g., with legs 734 distributed along at least that part of the perimeter. In some implementations, frame 730 is annular and circumscribes the perimeter of the downstream opening. Frame 730 may serve to maintain patency of pouch 710 in the presence of hemodynamic forces throughout the cardiac cycle.

In some implementations, legs 734 may be defined by frame 730—e.g., a single wire may be shaped to define both frame 730 and legs 734. In some implementations, the legs may be covered and/or encased by a flexible material. In some implementations, such a wire may also be shaped to form bends 732 that advantageously provide additional rigidity to frame 730. As shown in FIG. 52, in some examples, the bends 732 may not be covered by the flexible material. In some implementations, the bends 732 may be smaller in size than the legs 734. Alternatively, in some implementations one or more bends 732 may be larger in size than one or more legs 734. In some implementations, any or all of legs 734, frame 730, anchor 780, and/or pouch 710 may be covered in a tissue-growth-promoting material.

Augmenter 700 is configured to be anchored to a leaflet of the valve. In some implementations, as shown in FIG. 52, anchor 780 may protrude from the pouch 710 to anchor a leaflet. For example, anchor 780 may anchor the pouch to septal leaflet S, such that, responsively to ventricular systole, the augmenter is pushed atrial-ward in a manner that each of legs 734 is configured to tuck under a ventricular surface of one of the pair of opposing leaflets—e.g., anterior leaflet A and/or posterior leaflet P (FIG. 53A). Alternatively and/or additionally, in some implementations, the anchor 780 can be configured to anchor to the anterior leaflet A and/or posterior leaflet P. Legs 734 thus advantageously stabilize pouch 710 during ventricular systole by preventing prolapse of augmenter 700 into atrium 2.

In some implementations, a portion of the anchor 780 may be covered in a flexible material, as shown in FIG. 52. In some implementations, the anchor may be similar to other anchors described above and/or may not be covered in the flexible material.

In some implementations, augmenter 700 further comprises a support 740 that forks to define diverging limbs 742, 744, coupled to frame 730 on opposite sides of a perimeter of the frame (FIG. 52). In some applications, support 740 is coupled to anchor 780 (as shown) and/or to pouch 710 in the region of vent 750. Support 740 is shown with limbs 742 and 744 collectively defining a particular longitudinal plane p3 of augmenter 700 (e.g., on which anchor 780 also lies). Plane p3 may additionally or alternatively be defined as a longitudinal plane of the augmenter that passes through both of the radial positions on frame 730 at which limbs 742 and 744 are connected to the frame. it is to be understood that other orientations of the limbs are possible—e.g., such that the limbs collectively define and/or are connected to the frame on a different longitudinal plane, such as (but not limited to) a plane p4 that is orthogonal to plane p3.

In some implementations, support 740 may include more than two limbs. In some implementations, the limbs may be disposed at different radial positions on the frame 730, e.g., such that the two or more limbs are positioned on different longitudinal planes of the augmenter.

It is to be noted that upstream vent 750 and downstream legs 734 are independent features. In some implementations, augmenter 700 may comprise both features. In some implementations, augmenter 700 may comprise vent 750 without legs 734. In some other implementations, augmenter 700 may comprise legs 734 without vent 750. Similarly, vent 750 and legs 734 may be applied to other augmenters described herein and/or augmentors described in WO 2023/119065 to Ruban et al.

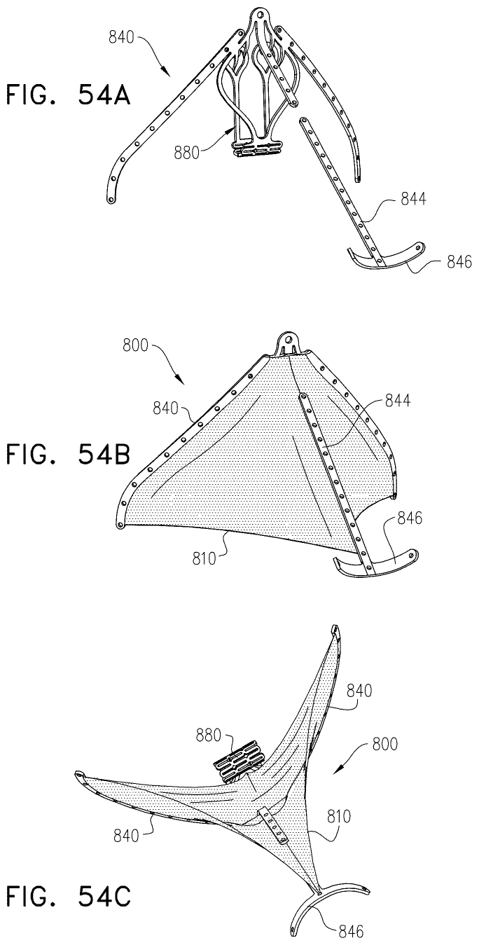

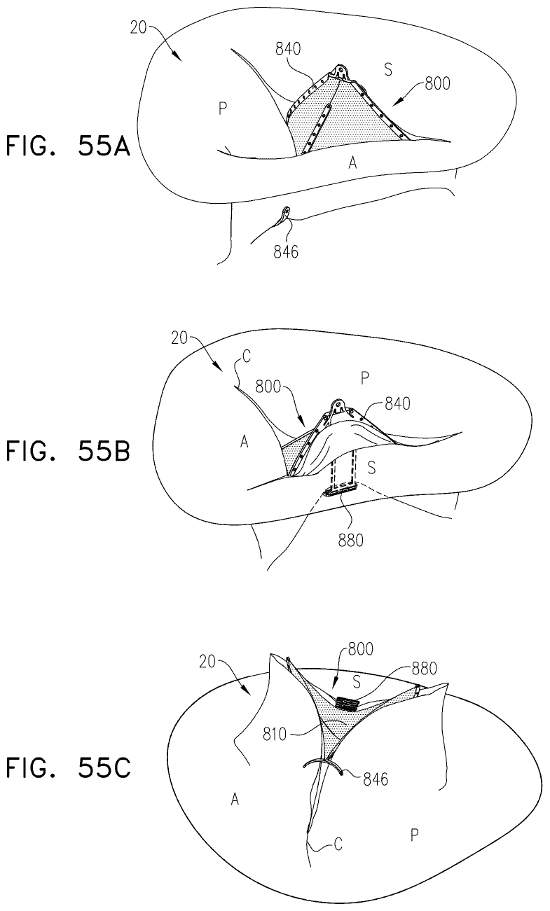

Reference is made to FIGS. 54A-C and 55A-C, which are schematic illustrations of a leaflet augmenter 800. FIGS. 54A-C show augmenter 800 from several isometric perspectives. FIGS. 55A-C show augmenter 800 implanted at a valve 20 of a heart 4, in accordance with some implementations. Valve 20 pictured in FIGS. 55A-C is a valve with three leaflets, e.g., the tricuspid valve. Where labeled, the leaflets are designated as S for septal, P for posterior, and A for anterior, respectively. In some implementations, augmenter 800 (e.g., variants thereof) can be used at a bicuspid valve such as the mitral valve, mutatis mutandis.

Augmenter 800 comprises a frame 840 and a pouch 810. Frame 840 comprises a set of supports coupled to an anchor 880. The set of supports may comprise three supports positioned to define an apex and three sides—e.g., approximating and/or defining a tetrahedral shape. In some implementations, frame 840 may be formed from a shape memory material. In some implementations, frame 840 and anchor 880 are cut from a single sheet of shape memory material. Frame 840 may be compressible away from its set shape and constrainable within a lumen of a delivery tool. In the set shape, the supports diverge from the apical region toward the base, e.g., to form the approximate tetrahedral shape.

In some implementations, augmenter 800 further comprises a leg 844 coupled to one of the supports of frame 840. Leg 844 is shaped to define a pair of distal grasps 846—e.g., at a downstream end of the leg. Grasps 846 may diverge away from each other—e.g., to collectively form a double-sided hook. In some implementations, a length of leg 844 may be adjustable to accommodate differences in the shape or size of valve 20 that may vary in individual subjects, as described in more detail below.

In some implementations, the leg 844 may be coupled to a support of the frame 840 such that the leg 844 diametrically opposes the anchor 840. In some implementations, the leg 844 may be coupled to a support of the frame 840, such that the leg 844 is adjacent to the anchor. In some implementations, two or more legs may be coupled to a respective support of the frame 840.

In some implementations, the leg 844 may be coupled to the frame 840 via a wire, a suture, a tether, or the like. In some implementations, the leg 844 may be coupled to the frame 840 via the pouch. In some implementations the leg 844 may be coupled to the frame 840 via one of the supports using one or more fasteners, sutures, wires, tethers, or the like.

Pouch 810 comprises a flexible or elastic material and is configured to be coupled to frame 840. Pouch 810 is configured to be stretched across frame 840 in a manner that shapes pouch 810 into a tetrahedral tent-like structure, such that the downstream opening is substantially triangular. During the cardiac cycle, the walls of pouch 810 are configured to billow in a manner that a downstream opening of pouch 810 transitions between a convex (e.g., Rouleau) triangle, and a concave (e.g., deltoid) triangle. During ventricular systole, the external surface billows outward to coapt with leaflets S, A, and P of valve 20, and during ventricular diastole, the external surface moves away from the leaflets of valve 20, thereby enabling blood flow through the valve. In some implementations, pouch 810 may be shaped to have an opening or vent at the apical region—e.g., as described for other augmenters herein.

Leg 844 protrudes away from at least one of the supports (FIGS. 54A-C) such that, responsively to ventricular systole, as the external surface coapts against the opposing leaflet, each of grasps 846 tucks under a respective opposing leaflet of the pair of opposing leaflets A, P (FIGS. 55A-C). This may be viewed as leg 844 engaging and/or tucking under a commissure C common to the two opposing leaflets. In this manner, the grasps may prevent upstream pivoting of the pouch 810 into the atrium.

Augmenter 800 may be particularly advantageous for subjects with septal leaflet pathology. Augmenter 800 may be configured to billow open during systole and deflate during diastole. The configuration of augmenter 800 advantageously keeps pouch 810 away from the ventricular wall and avoids interaction with the tricuspid valve annulus, thus reducing potential irritation of electrically conductive nodal tissue that could otherwise contribute to cardiac arrythmias.

It is to be noted that leg 844 and/or grasps 846 may be added to any of the other augmenters described herein.

The systems, apparatuses, and techniques described herein can be used in combination with those described in one or more of the following references, each of which is incorporated herein by reference in its entirety. For example, techniques described in one or more of the following references, each of which is incorporated by reference herein for all purposes, for attachment to ventricular tissue and/or valve leaflets, and/or for adjusting tension of a tether (e.g., a prosthetic chorda tendinea), can be used in combination with one or more of the apparatus and techniques described herein. Similarly, any of the augmenters described herein can be used as a component of an implant (e.g., as a “patch” or “upstream assembly” of the implant) described in any of the following references:
International Patent Publication WO 2022/101817 to Tennenbaum et al.;
U.S. Patent Application 63/113,430 to Tennenbaum et al., filed Nov. 13, 2020;
U.S. Pat. No. 9,277,994 to Miller et al.;
U.S. Pat. No. 9,180,007 to Reich et al.

Any of the various systems, assemblies, devices, components, apparatuses, etc. in this disclosure can be sterilized (e.g., with heat, radiation, ethylene oxide, hydrogen peroxide, etc.) to ensure they are safe for use with patients, and the methods herein can comprise (or additional methods comprise or consist of) sterilization of the associated system, device, component, apparatus, etc. (e.g., with heat, radiation, ethylene oxide, hydrogen peroxide, etc.). Furthermore, the scope of the present disclosure includes, in some implementations, sterilizing one or more of any of the various systems, devices, apparatuses, etc. in this disclosure.

The techniques, methods, operations, steps, etc. described or suggested herein or in the references incorporated herein can be performed on a living subject (e.g., human, other animal, etc.) or on a simulation, such as a cadaver, cadaver heart, simulator, imaginary person, etc. When performed on a simulation, the body parts, e.g., heart, tissue, valve, etc., can be assumed to be simulated or can optionally be referred to as “simulated” (e.g., simulated heart, simulated tissue, simulated valve, etc.) and can optionally comprise computerized and/or physical representations of body parts, tissue, etc. The term “simulation” covers use on a cadaver, computer simulator, imaginary person (e.g., if they are just demonstrating in the air on an imaginary heart), etc.

### EXAMPLE IMPLEMENTATIONS (SOME NON-LIMITING EXAMPLES OF THE CONCEPTS HEREIN ARE RECITED BELOW)

Example 1. An apparatus, useable with a valve disposed between an atrium and a ventricle of a heart, the valve having a first leaflet and an opposing leaflet, the first leaflet having a lip, the apparatus comprising an augmenter that comprises: (A) a flexible pouch, having an external surface, an interior, and a downstream opening that provide fluid communication between the interior and an outside of the pouch; and/or (B) an anchor, coupled to the pouch, and configured to anchor the pouch to the lip of the first leaflet by the anchor being anchored to the first leaflet, such that: (i) the downstream opening opens into the ventricle, and/or (ii) responsively to ventricular systole: (1) the pouch inflates via the downstream opening; and/or (2) the external surface coapts against the opposing leaflet.

Example 2. The apparatus according to example 1, wherein the pouch is rectangular.

Example 3. The apparatus according to any one of examples 1-2, wherein the pouch is mitre-shaped.

Example 4. The apparatus according to any one of examples 1-3, wherein the pouch is conical.

Example 5. The apparatus according to any one of examples 1-4, wherein the pouch is pillow-shaped.

Example 6. The apparatus according to any one of examples 1-5, wherein the pouch is shaped as a rounded square.

Example 7. The apparatus according to any one of examples 1-6, wherein the pouch is globular.

Example 8. The apparatus according to any one of examples 1-7, wherein the pouch is pyramidal.

Example 9. The apparatus according to any one of examples 1-7, wherein the pouch is tetrahedral.

Example 10. The apparatus according to any one of examples 1-8, wherein the augmenter is sterile.

Example 11. The apparatus according to any one of examples 1-10, wherein the anchor comprises a clip.

Example 12. The apparatus according to any one of examples 1-11, wherein the anchor comprises a pair of clips.

Example 13. The apparatus according to any one of examples 1-12, wherein: (A) the first leaflet has a root attached to an annulus of the valve; and/or (B) the anchor is configured to be anchored to the first leaflet closer to the lip than to the root.

Example 14. The apparatus according to any one of examples 1-13, wherein the augmenter comprises a film from which the pouch is formed.

Example 15. The apparatus according to any one of examples 1-14, wherein the augmenter comprises a polymer from which the pouch is formed.

Example 16. The apparatus according to any one of examples 1-15, wherein the anchor is configured to anchor the pouch to the lip of the first leaflet such that the pouch deflates responsively to ventricular diastole.

Example 17. The apparatus according to any one of examples 1-16, wherein: (A) the opposing leaflet is a first opposing leaflet, (B) the valve is a tricuspid valve having a second opposing leaflet, and/or (C) the anchor is configured to anchor the pouch to the lip of the first leaflet such that responsively to ventricular systole, the pouch inflates via the downstream opening, such that the external surface coapts against both the first opposing leaflet and the second opposing leaflet.

Example 18. The apparatus according to example 17, wherein the anchor is configured to anchor the pouch to the lip of the first leaflet such that, responsively to ventricular systole, the pouch inflates via the downstream opening, such that at least part of the pouch interposes between the first opposing leaflet and the second opposing leaflet.

Example 19. The apparatus according to any one of examples 1-18, wherein the anchor is selected from: tissue-piercing elements, T-bars, threads, sutures, pins, clips, and locking elements.

Example 20. The apparatus according to any one of examples 1-19, further comprising a stem that couples the pouch to the anchor by extending: (A) from the anchor, outside of the pouch toward the downstream opening, (B) under a rim of the downstream opening, thereby defining a bight of the stem, and/or (C) from the bight, through the interior to an upstream apex of the pouch, thereby defining a mast of the augmenter.

Example 21. The apparatus according to any one of examples 1-20, wherein the augmenter comprises an adjustment mechanism that is actuatable to adjust positioning between the anchor and the pouch.

Example 22. The apparatus according to any one of examples 1-21, wherein the anchor is coupled to the pouch in a manner that facilitates adjustment of positioning between the anchor and the pouch.

Example 23. The apparatus according to any one of examples 1-22, wherein the augmenter is configured to facilitate adjustment of positioning between the anchor and the pouch while the pouch remains anchored to the lip by the anchor, in a manner that changes a proportion of the pouch that is disposed within the atrium during ventricular systole.

Example 24. The apparatus according to any one of examples 1-23, further comprising a stem via which the pouch is adjustably coupled to the anchor.

Example 25. The apparatus according to any one of examples 1-24, wherein: (A) the valve is a tricuspid valve; (B) the first leaflet is a septal leaflet of the tricuspid valve; and/or (C) the anchor is configured to anchor the pouch to the lip of the septal leaflet by being anchored to the septal leaflet.

Example 26. The apparatus according to any one of examples 1-25, wherein the augmenter further comprises an adjustable strap coupled to the pouch such that adjusting a length of the strap adjusts an internal dimension of the pouch.

Example 27. The apparatus according to example 26, wherein the adjustable strap spans the interior of the pouch.

Example 28. The apparatus according to example 26, wherein the adjustable strap spans the downstream opening.

Example 29. The apparatus according to example 26, wherein the adjustable strap lies parallel to the internal dimension.

Example 30. The apparatus according to example 26, wherein: (A) the opposing leaflet is a first opposing leaflet, (B) the valve is a tricuspid valve having a second opposing leaflet, and/or (C) the adjustable strap is configured to reshape the external surface to increase coaptation, during ventricular systole, between the external surface and the first and second opposing leaflets.

Example 31. The apparatus according to example 26, wherein the adjustable strap comprises a fabric.

Example 32. The apparatus according to example 26, wherein the adjustable strap is an adjustable strap selected from: a belt, a cord, a line, a thread, a braid, a thong, a tape, a tie, a cable, a wire, an elastic, and a ligature.

Example 33. The apparatus according to example 26, wherein the adjustable strap comprises a belt with a buckle.

Example 34. The apparatus according to example 26, wherein the adjustable strap comprises a ratchet.

Example 35. The apparatus according to example 26, wherein the adjustable strap comprises a locking mechanism.

Example 36. The apparatus according to example 26, wherein the adjustable strap comprises a hook and loop self-fastener.

Example 37. The apparatus according to example 26, wherein decreasing a length of the adjustable strap limits inflation of the pouch.

Example 38. The apparatus according to example 26, wherein the adjustable strap is a first adjustable strap, and the augmenter further comprises a second adjustable strap.

Example 39. The apparatus according to example 38, wherein the pouch has a longitudinal plane, the first adjustable strap and the second adjustable strap being disposed on opposite sides of the longitudinal plane from each other.

Example 40. The apparatus according to example 39, wherein the first adjustable strap lies parallel with the longitudinal plane.

Example 41. The apparatus according to example 39, wherein the first adjustable strap lies parallel with the second adjustable strap.

Example 42. The apparatus according to example 39, wherein the longitudinal plane bisects the downstream opening.

Example 43. The apparatus according to example 39, wherein the anchor is disposed on the longitudinal plane.

Example 44. The apparatus according to example 38, wherein the pouch has a transverse plane, the first adjustable strap and the second adjustable strap being disposed on opposite sides of the transverse plane from each other.

Example 45. The apparatus according to example 44, wherein the first adjustable strap lies parallel with the transverse plane.

Example 46. The apparatus according to example 44, wherein the first adjustable strap lies parallel with the second adjustable strap.

Example 47. The apparatus according to example 44, wherein: (A) the first adjustable strap is disposed on an upstream side of the transverse plane, and/or (B) the second adjustable strap and the downstream opening are disposed on a downstream side of the transverse plane.

Example 48. The apparatus according to example 44, wherein the anchor is disposed on the transverse plane.

Example 49. The apparatus according to any one of examples 1-48, wherein the external surface defines a central opening that provides fluid communication between the interior and the outside of the pouch.

Example 50. The apparatus according to any one of examples 1-49, wherein the augmenter comprises a fabric from which the pouch is formed.

Example 51. The apparatus according to example 50, wherein the pouch comprises and/or is formed by three-dimensional weaving of the fabric.

Example 52. The apparatus according to example 50, wherein the pouch comprises and/or is formed by three-dimensional knitting of the fabric.

Example 53. The apparatus according to any one of examples 1-52, wherein the augmenter comprises a polymer from which the pouch is formed.

Example 54. The apparatus according to example 53, wherein the pouch comprises and/or is formed by three-dimensional weaving of the polymer.

Example 55. The apparatus according to example 53, wherein the pouch comprises and/or is formed by three-dimensional knitting of the polymer.

Example 56. The apparatus according to any one of examples 1-55, wherein: (A) the augmenter has a ceiling at an opposite end of the augmenter from the downstream opening, and/or (B) the anchor is configured to anchor the augmenter to the lip of the first leaflet such that the downstream opening is downstream of the ceiling.

Example 57. The apparatus according to example 56, wherein the anchor is configured to anchor the augmenter to the lip of the first leaflet such that the ceiling is disposed in the atrium during ventricular systole.

Example 58. The apparatus according to any one of examples 1-57, wherein the augmenter further comprises: (A) a frame to which the pouch and the anchor are coupled; and/or (B) a stabilizer, coupled to the frame, extending away from the frame to a projection of the stabilizer, the stabilizer being configured to be positioned with the projection at a site in the heart in a manner that resists upstream pivoting of the pouch.

Example 59. The apparatus according to example 58, wherein: (A) the stabilizer comprises a resilient wire, and/or (B) the anchor is configured to anchor the pouch to the lip of the first leaflet such that the resilient wire extends in an arc upstream of the pouch and presses the projection into a commissure of the valve.

Example 60. The apparatus according to example 59, wherein the resilient wire defines a bowed spring.

Example 61. The apparatus according to example 59, wherein the projection is shaped to hook against a downstream surface of the commissure.

Example 62. The apparatus according to example 58, wherein: (A) the frame defines a mast, (B) the projection is configured to be driven into heart tissue upstream of the valve, and/or (C) the stabilizer comprises a tether that tethers the mast to the projection such that the tether becomes tensioned during ventricular systole.

Example 63. The apparatus according to example 62, wherein the projection comprises a tissue anchor.

Example 64. The apparatus according to example 58, wherein the projection comprises a foil that has a shape and an angle of attack that generates lift in a manner that resists the upstream pivoting.

Example 65. The apparatus according to example 64, wherein the tether, by utilizing hemodynamic forces, traverses a pulmonary valve of the heart such that the foil is disposed within a pulmonary artery of the heart.

Example 66. The apparatus according to example 64, wherein the tether, by utilizing hemodynamic forces, is disposed within the ventricle.

Example 67. The apparatus according to example 58, wherein: (A) the ventricle has a septal wall, (B) the stabilizer comprises a tether, and/or (C) the projection comprises a tissue anchor configured to be anchored to the septal wall in a manner that limits atrial pivoting of the pouch during ventricular contraction.

Example 68. The apparatus according to example 67, wherein the tissue anchor is configured to be anchored to the septal wall by traversing the septal wall.

Example 69. The apparatus according to example 58, wherein: (A) the projection comprises a resilient spring, and/or (B) the anchor is configured to anchor the pouch to the lip of the first leaflet in a manner that places the projection against a wall of the atrium.

Example 70. The apparatus according to example 58, wherein: (A) the stabilizer comprises a bowed spring, and/or (B) the augmenter is configured to be positioned such that the bowed spring presses against a wall of the ventricle.

Example 71. The apparatus according to example 70, wherein the bowed spring defines the projection, and the stabilizer further comprises a tether that tethers the bowed spring to the frame.

Example 72. The apparatus according to example 70, wherein the bowed spring is shaped to extend circumferentially around the ventricle and to press radially outward against the wall of the ventricle.

Example 73. The apparatus according to example 58, wherein the stabilizer comprises a tether that tethers the projection to the frame.

Example 74. The apparatus according to example 58, wherein the stabilizer comprises a resilient wire that couples the projection to the frame.

Example 75. The apparatus according to example 58, wherein the projection comprises an expandable stent, configured to be secured within a cardiovascular lumen by being expanded within the cardiovascular lumen.

Example 76. The apparatus according to example 75, wherein the stabilizer comprises a tether that tethers the projection to the frame.

Example 77. The apparatus according to example 75 wherein the projection is configured to be secured within a superior vena cava.

Example 78. The apparatus according to example 75, wherein the projection is configured to be secured within an inferior vena cava.

Example 79. The apparatus according to example 75, wherein the projection is configured to be secured within a pulmonary artery.

Example 80. The apparatus according to example 75, wherein the projection is configured to be secured within a coronary sinus.

Example 81. The apparatus according to any one of examples 1-80, wherein:
the first leaflet is attached to chordae tendineae of the heart; and
the augmenter further comprises a stabilizer, coupled to the pouch, and configured to be looped behind chordae tendineae of the first leaflet in a manner that resists upstream pivoting of the pouch.

Example 82. The apparatus according to example 81, wherein the augmenter further comprises a frame to which the pouch and the anchor are coupled, the stabilizer being coupled to the pouch by being coupled to the frame.

Example 83. The apparatus according to example 81, wherein the stabilizer comprises a rod, and is configured to be looped behind the chordae tendineae of the first leaflet in a manner that positions the rod behind the chordae tendineae.

Example 84. The apparatus according to example 83, wherein the stabilizer comprises a wire that is coupled to the rod and that has a length that is adjustable in a manner that adjusts the resisting of the upstream pivoting of the pouch.

Example 85. The apparatus according to example 83, wherein the pouch has a transverse plane disposed between a proximal end of the pouch and the downstream opening, and the stabilizer is configured to be looped behind the chordae tendineae of the first leaflet in a manner that positions the rod behind the chordae tendineae, parallel to the transverse plane.

Example 86. The apparatus according to example 85, wherein the stabilizer is configured to be looped behind the chordae tendineae of the first leaflet such that the rod when positioned behind the chordae tendineae, extends laterally beyond the pouch parallel to the transverse plane.

Example 87. The apparatus according to any one of examples 1-86, wherein the pouch is shaped to define a vent at an upstream region of the pouch, the vent configured to allow limited retrograde blood flow therethrough.

Example 88. The apparatus according to example 87, wherein the vent is one of multiple vents at the proximal end of the pouch.

Example 89. The apparatus according to example 88, wherein the multiple vents are disposed on an opposite side of the pouch to the anchor.

Example 90. The apparatus according to example 87, wherein the pouch is conical and defines an apex, the vent being disposed at the apex.

Example 91. The apparatus according to example 90, wherein the vent comprises a grommet that delimits the vent.

Example 92. The apparatus according to example 90, wherein the vent comprises a check valve that is oriented to allow the limited retrograde blood flow through the vent and to inhibit antegrade blood flow through the vent.

Example 93. The apparatus according to example 92, wherein the check valve is a duckbill valve.

Example 94. The apparatus according to example 92, wherein the check valve is a reed valve.

Example 95. The apparatus according to example 92, wherein the check valve is a diaphragm valve.

Example 96. The apparatus according to any one of examples 1-95, further comprising a resilient wire, coupled to an upstream part of the pouch in a manner that squeezes blood out of the upstream part of the pouch during diastole.

Example 97. The apparatus according to any one of examples 1-96, wherein: (A) the valve is a tricuspid valve having the first leaflet, a second leaflet, and the opposing leaflet; (B) the anchor is a first anchor, and is coupled to the pouch at a first face of the pouch; (C) the augmenter further comprises a second anchor, coupled to the pouch at a second face of the pouch, the external surface being an external surface of a third face of the pouch; and
the first and second anchors are configured to anchor the first and second faces, respectively, to the first and second leaflets, respectively, such that, responsively to ventricular systole, the external surface of the third face of the pouch coapts against the opposing leaflet.

Example 98. The apparatus according to example 97, wherein the apparatus further comprises a frame internal to the pouch to which the first and second anchors are fixedly coupled.

Example 99. The apparatus according to example 97, wherein the pouch is dimensioned and coupled to the frame, such that, upon anchoring of the first and second faces to the first and second leaflets by the first and second anchors, the pouch projects into the ventricle.

Example 100. The apparatus according to example 97, wherein the pouch is dimensioned and coupled to the frame, such that, upon anchoring of the first and second faces to the first and second leaflets by the first and second anchors, the pouch projects into the atrium.

Example 101. The apparatus according to any one of examples 1-100, further comprising: a frame to which the pouch and the anchor are fixedly coupled, the frame comprising: (i) a hinge; and/or (ii) a tube slidably coupled to the hinge such that sliding the tube over the hinge inactivates the hinge.

Example 102. The apparatus according to example 101, wherein the hinge is configured to confer flexibility on a segment of the frame, and sliding the tube over the hinge stiffens the segment.

Example 103. The apparatus according to example 101, wherein the tube is configured to lock into place after sliding over the hinge.

Example 104. The apparatus according to any one of examples 1-103, wherein: (A) the augmenter comprises a frame to which the anchor is coupled, the anchor comprising an upstream clasp element and a downstream clasp element hingedly coupled to the frame; and/or (B) the apparatus comprises a delivery tool configured to: (i) transluminally advance the augmenter to the valve, (ii) position the downstream clasp element at a downstream side of the first leaflet by passing the downstream clasp element between chordae tendineae of the first leaflet, (iii) subsequently, and at the downstream side of the first leaflet, actuating the downstream clasp element to expand laterally, and/or (iv) anchor the anchor to the first leaflet by clasping the first leaflet between (1) the laterally-expanded downstream clasp element against the downstream side of the first leaflet, and/or (2) the upstream clasp element against an upstream side of the first leaflet.

Example 105. The apparatus according to example 104, wherein: (A) the downstream clasp element is restrained laterally by the delivery tool until deployment, and/or (B) the delivery tool is configured to actuate the downstream clasp element by releasing the downstream clasp element to expand.

Example 106. The apparatus according to example 104, wherein the delivery tool is configured to actuate the downstream clasp element by pulling on the downstream clasp element.

Example 107. The apparatus according to example 104, wherein the delivery tool is configured to actuate the downstream clasp element by pushing on the downstream clasp element.

Example 108. The apparatus according to example 104, wherein the delivery tool is configured to actuate the downstream clasp element by unsheathing the downstream clasp element.

Example 109. The apparatus according to any one of examples 1-108, wherein the apparatus further comprises a stabilizer, coupled to the anchor, extending away from the anchor and the pouch, and shaped such that, when the anchor is anchored to the first leaflet, the stabilizer rests on a tissue surface of the atrium.

Example 110. The apparatus according to example 109, wherein: (A) the valve is a tricuspid valve; (B) the first leaflet is a septal leaflet of the tricuspid valve; and/or (C) the stabilizer comprises two curved arms configured to extend in opposite directions from a point in the atrium above the anchoring of the anchor to the first leaflet, in a manner that the curved arms are configured to rest on a septal wall of the atrium above an atrioventricular node of the heart.

Example 111. The apparatus according to example 109, wherein the stabilizer comprises a loop coupled to a rod, the rod coupled to the anchor and configured to be positioned over the first leaflet, and the loop configured to rest on an annulus of the valve.

Example 112. The apparatus according to example 111, further comprising a stabilizer spring configured to: (A) couple the rod to the stem, and/or (B) facilitate lateral flexibility of the pouch.

Example 113. The apparatus according to any one of examples 1-112, wherein the pouch is shaped to have a base at which the downstream opening is located, and an apical region that that includes an apex, the pouch tapering from the base toward the apex.

Example 114. The apparatus according to example 113, wherein the pouch is conical.

Example 115. The apparatus according to example 114, wherein the downstream opening is substantially elliptical.

Example 116. The apparatus according to example 113, wherein the pouch is tetrahedral.

Example 117. The apparatus according to example 116, wherein the downstream opening is substantially triangular.

Example 118. The apparatus according to example 116, further comprising a set of supports: (A) diverging from the apical region toward the base; and/or (B) coupled to the pouch in a manner that shapes the pouch to be tetrahedral.

Example 119. The apparatus according to example 118, wherein the set of supports is coupled to the anchor.

Example 120. The apparatus according to example 118, wherein the augmenter further comprises a leg coupled to, and protruding away from, at least one of the supports such that, responsively to ventricular systole, as the external surface coapts against the opposing leaflet, the leg tucks under the opposing leaflet.

Example 121. The apparatus according to example 120, wherein: (A) the opposing leaflet is a first opposing leaflet of a pair of opposing leaflets, and/or (B) the leg is shaped to define a pair of grasps that diverge from each other such that, responsively to ventricular systole, as the external surface coapts against the opposing leaflet each of the grasps of the leg tucks under a respective opposing leaflet of the pair of opposing leaflets.

Example 122. The apparatus according to example 120, wherein a length of the leg is adjustable.

Example 123. The apparatus according to example 120, wherein: (A) the opposing leaflet is a first opposing leaflet of a pair of opposing leaflets, and/or (B) the leg is shaped to define a pair of grasps that diverge from each other such that, responsively to ventricular systole, as the external surface coapts against the opposing leaflet the grasps of the leg engage a commissure at which the pair of opposing leaflets meet.

Example 124. The apparatus according to example 113, wherein the augmenter further comprises a leg protruding externally from the pouch and away from the downstream opening such that, responsively to ventricular systole, as the external surface coapts against the opposing leaflet, the leg tucks under the opposing leaflet.

Example 125. The apparatus according to example 124, wherein the leg is a first leg and wherein the apparatus comprises multiple legs.

Example 126. The apparatus according to example 125, wherein at least some of the legs are covered by a growth-promoting material.

Example 127. The apparatus according to example 125, wherein: (A) the opposing leaflet is a first opposing leaflet, the valve comprising a pair of opposing leaflets, and/or (B) each of the multiple legs is configured to tuck under one of the pair of opposing leaflets.

Example 128. The apparatus according to example 125, wherein: (A) the augmenter comprises a frame that is coupled to the pouch along at least part of a perimeter of the downstream opening, and/or (B) the multiple legs are defined by the frame and are distributed around at least the part of the perimeter.

Example 129. The apparatus according to example 128, further comprising a support that is coupled to the anchor, and that forks to define diverging limbs that are coupled to the frame on opposing sides of the perimeter.

Example 130. The apparatus according to example 128, wherein the frame is annular and circumscribes the perimeter of the downstream opening.

Example 131. The apparatus according to example 113, wherein the pouch is configured with a vent at the apex.

Example 132. The apparatus according to example 131, wherein the vent is one of multiple vents at the apical region.

Example 133. The apparatus according to example 131, wherein the vent comprises a grommet that delimits the vent.

Example 134. The apparatus according to example 131, wherein the vent comprises a check valve that is oriented to allow the limited retrograde blood flow through the vent and to inhibit antegrade blood flow through the vent.

Example 135. The apparatus according to example 134, wherein the check valve is a duckbill valve.

Example 136. The apparatus according to example 134, wherein the check valve is a reed valve.

Example 137. The apparatus according to example 134, wherein the check valve is a diaphragm valve.

Example 138. The apparatus according to example 113, wherein: (A) the flexible pouch comprises and/or is formed from a fabric; and/or (B) the pouch comprises at least one wire that has a stiffness greater than that of the fabric, and that extends, in association with the fabric, from an apical vent toward the downstream opening.

Example 139. The apparatus according to example 138, wherein the at least one wire includes at least three wires, distributed circumferentially around the pouch, such that the at least three wires deflect away from each other as the pouch inflates.

Example 140. The apparatus according to example 113, further comprising a stem that couples the pouch to the anchor in a manner that enables adjustment of a position of the pouch with respect to the anchor.

Example 141. The apparatus according to example 140, wherein the augmenter is configured such that, when the anchor is anchored to the first leaflet, progressive adjustment of the position of the pouch atrial-ward positions progressively wider regions of the pouch between the first leaflet and the opposing leaflet.

Example 142. The apparatus according to example 140, wherein, when the anchor is anchored to the lip of the first leaflet, progressive adjustment of the position of the pouch ventricle-ward positions progressively narrower regions of the pouch between the first leaflet and the opposing leaflet.

Example 143. The apparatus according to example 140, wherein the stem is axially fixed with respect to the pouch, and threadedly coupled to the anchor.

Example 144. The apparatus according to example 140, wherein the stem is axially fixed with respect to the anchor, and threadedly coupled to the pouch.

Example 145. The apparatus according to example 140, wherein: (A) the augmenter further comprises a sleeve, fixed to the anchor, and/or (B) the stem is fixed to the pouch, and couples the pouch to the anchor by being disposed through the sleeve.

Example 146. The apparatus according to example 140, wherein the augmenter further comprises a sleeve having internal threads, and the stem is coupled to: (A) the pouch via a rotary bearing at either end of the stem; and/or (B) the anchor by being disposed through the sleeve, the stem having external threads such that rotating the stem within the sleeve adjusts a position of the pouch along the sleeve.

Example 147. The apparatus according to example 140, wherein the augmenter further comprises a sleeve, and the stem is coupled to: (A) the anchor by being disposed through the sleeve in a manner that defines a rotary bearing, and/or (B) the pouch via at least one threaded nut, such that rotating the stem within the at least one threaded nut adjusts a position of the pouch along the stem.

Example 148. The apparatus according to example 140, wherein: (A) the stem is coupled to the pouch at the apical region of the pouch, and/or (B) the augmenter further comprises a threaded nut, at the apical region of the pouch, configured such that rotating the threaded nut adjusts a position of the pouch along the stem.

Example 149. The apparatus according to example 148, wherein the augmenter further comprises an adjustment spring, arranged with respect to the threaded nut such that rotating the threaded nut in a first rotational direction strains the spring, and rotating the threaded nut in an opposite rotational direction relaxes the spring.

Example 150. The apparatus according to example 149, wherein the adjustment spring is disposed coaxially around the stem within the interior of the pouch.

Example 151. The apparatus according to example 140, wherein the stem extends: (A) from the anchor toward the downstream opening, (B) under a rim of the downstream opening, thereby defining a bight of the stem, and/or (C) from the bight, through the interior to the apical region, thereby defining a mast of the augmenter, the mast being coupled to the pouch at the apical region.

Example 152. The apparatus according to example 140, wherein: (A) the augmenter further comprises a sleeve, fixed to the anchor, and/or (B) the stem couples the pouch to the anchor by being disposed through the sleeve, and enables adjustment of the position of the pouch with respect to the anchor via sliding of the stem through the sleeve.

Example 153. The apparatus according to example 152, wherein: (A) the stem comprises a rod that has ends that are fixed to the pouch, and a midsection, between the ends; and/or (B) progressively sliding the midsection through the sleeve in an atrial-ward direction positions progressively wider regions of the pouch between the first leaflet and the opposing leaflet.

Example 154. The apparatus according to example 152, wherein: (A) the stem comprises a rod that has ends that are fixed to the pouch, and a midsection, between the ends; and/or (B) progressively sliding the midsection through the sleeve in a ventricle-ward direction positions progressively narrower regions of the pouch between the first leaflet and the opposing leaflet.

Example 155. The apparatus according to example 152, wherein the stem has: (A) a first region that is slidable through the sleeve, and/or (B) a second region fixed to the apical region of the pouch, the second region being stiffer than the first region.

Example 156. The apparatus according to example 155, wherein the augmenter is configured such that, when the anchor is anchored to the first leaflet, progressively sliding the first region through the sleeve in an atrial-ward direction positions progressively narrower regions of the pouch between the first leaflet and the opposing leaflet.

Example 157. The apparatus according to example 155, wherein the augmenter is configured such that, when the anchor is anchored to the first leaflet, progressively sliding the first region through the sleeve in a ventricle-ward direction positions progressively wider regions of the pouch between the first leaflet and the opposing leaflet.

Example 158. The apparatus according to example 155, wherein the augmenter is configured such that, when the anchor is anchored to the first leaflet, sliding the first region through the sleeve in an atrial-ward direction moves the pouch in a ventricle-ward direction.

Example 159. The apparatus according to example 155, wherein the augmenter is configured such that, when the anchor is anchored to the first leaflet, sliding the first region through the sleeve in a ventricle-ward direction moves the pouch in an atrial-ward direction.

Example 160. The apparatus according to example 155, wherein the augmenter further comprises a threaded nut disposed at an atrial region of the sleeve, configured such that rotating the threaded nut adjusts a position of the stem along the sleeve.

Example 161. The apparatus according to example 140, wherein the stem: (A) has a first part that extends from the anchor toward the downstream opening, (B) has a second part that defines a mast that extends through the interior to the apical region, the mast being coupled to the pouch at the apical region, and/or (C) comprises an adaptive joint that couples the first part to the second part in a manner that facilitates passive reshaping of the stem responsively to intracardial forces.

Example 162. The apparatus according to example 161, wherein the adaptive joint comprises a ball-and-socket joint.

Example 163. The apparatus according to example 161, wherein the adaptive joint comprises a floating joint configured to facilitate lateral adjustability of the pouch.

Example 164. The apparatus according to example 161, wherein the adaptive joint comprises a hinge.

Example 165. The apparatus according to example 140, wherein the stem defines a mast that extends through the interior to the apical region, the mast being coupled to the pouch at the apical region via a ratchet mechanism that enables adjustment of the position by ratcheting the mast through the apical region.

Example 166. The apparatus according to example 165, wherein the ratchet mechanism comprises a set of teeth arranged along the mast, and at least one pawl at the apical region, enabling adjustment of the position by ratcheting the teeth through the apical region.

Example 167. The apparatus according to example 165, wherein, when the augmenter is anchored to the lip of the first leaflet, ratcheting the mast through the apical region moves the pouch atrial-ward.

Example 168. The apparatus according to example 165, wherein, when the augmenter is anchored to the lip of the first leaflet, ratcheting the mast though the apical region moves the pouch ventricular-ward.

Example 169. An apparatus, useable with a valve of a heart, the valve having a first leaflet and a second leaflet, the first leaflet having a lip, the apparatus comprising an augmenter that comprises: (A) a frame; (B) an anchor, coupled to the frame, and configured to anchor the augmenter to the lip of the first leaflet; and/or (C) a panel comprising a flexible sheet: (i) having a first surface that faces the frame and a second surface that faces away from the frame, and/or (ii) coupled to the frame in a manner that provides a billow zone at which the flexible sheet is unattached to the frame, thereby facilitating the panel billowing away from the frame responsively to fluid pressure at the first surface.

Example 170. The apparatus according to example 169, wherein the augmenter is sterile.

Example 171. The apparatus according to any one of examples 169-170, wherein: (A) the billow zone is a first billow zone, (B) the first billow zone facilitates the flexible sheet, at the first billow zone, billowing away from the frame in a first direction responsively to the fluid pressure, and/or (C) the flexible sheet is coupled to the frame so as to further define at least a second billow zone that facilitates the flexible sheet, at the second billow zone, billowing away from the frame in a second direction responsively to the fluid pressure, the second direction being opposite to the first direction.

Example 172. The apparatus according to any one of examples 169-171, wherein the panel is coupled to the frame by being secured at a plurality of points on the frame.

Example 173. The apparatus according to any one of examples 169-172, wherein the panel is coupled to the frame by being secured at a plurality of peripheral points, but not at a mid-region, of the panel.

Example 174. The apparatus according to any one of examples 169-173, wherein at least one lateral edge of the panel is unattached to the frame.

Example 175. The apparatus according to any one of examples 169-174, wherein the augmenter is configured such that the billowing of the panel enhances coaptation between the second surface and the second leaflet during systole.

Example 176. The apparatus according to any one of examples 169-175, wherein the anchor is fixedly coupled to the frame.

Example 177. The apparatus according to any one of examples 169-176, wherein the anchor is pivotably coupled to the frame.

Example 178. The apparatus according to any one of examples 169-177, wherein the anchor is a pair of clips.

Example 179. The apparatus according to any one of examples 169-178, wherein: (A) the first leaflet has a root, the root being attached to an annulus of the valve; and/or (B) the anchor is configured to be anchored to the first leaflet closer to the lip than to the root.

Example 180. The apparatus according to any one of examples 169-179, wherein the flexible sheet comprises a film.

Example 181. The apparatus according to any one of examples 169-180, wherein the augmenter is configured to billow during systole and to collapse during diastole, in an oscillating manner with each cardiac cycle.

Example 182. The apparatus according to any one of examples 169-181, wherein the frame comprises a shape-memory alloy.

Example 183. The apparatus according to any one of examples 169-182, wherein: (A) at one end of the augmenter, the augmenter has a root portion, (B) at an opposite end of the augmenter, the augmenter has a lip portion, and/or (C) the anchor is configured to anchor the augmenter to the lip of the first leaflet such that the lip portion is downstream of the root portion.

Example 184. The apparatus according to any one of examples 169-183, wherein, at the lip portion, the panel is attached to the frame along an edge of the panel.

Example 185. The apparatus according to any one of examples 169-184, wherein, at the root portion, the anchor is coupled to the frame.

Example 186. The apparatus according to any one of examples 169-185, wherein, at a central portion of the augmenter, the anchor is coupled to the frame, the central portion being partway between the root portion and the lip portion.

Example 187. The apparatus according to any one of examples 169-186, wherein the anchor is a clip.

Example 188. The apparatus according to any one of examples 169-187, wherein

the frame and the clip are formed from a single piece of stock material.

Example 189. The apparatus according to any one of examples 169-188, wherein the flexible sheet comprises a fabric.

Example 190. The apparatus according to any one of examples 169-189, wherein the fabric comprises a woven polymer.

Example 191. The apparatus according to any one of examples 169-190, wherein the fabric comprises a knitted polymer.

Example 192. The apparatus according to any one of examples 169-191, wherein the panel is a first panel extending over a first side of the frame, and wherein the augmenter further comprises a second panel, the second panel extending over an opposite side of the frame, the second panel having a central opening therethrough.

Example 193. The apparatus according to example 192, wherein the central opening provides fluid communication with the first surface such that the first panel experiences the fluid pressure via the central opening, and responsively billows.

Example 194. The apparatus according to example 192, wherein the central opening is substantially central with respect to the second panel.

Example 195. The apparatus according to any one of examples 169-194, wherein the first panel and the second panel are attached to each other along at least one edge in a manner that defines a cavity.

Example 196. The apparatus according to any one of examples 169-195, wherein: (A) at one end of the augmenter, the augmenter has a root portion, (B) at an opposite end of the augmenter, the augmenter has a lip portion, (C) the anchor is configured to anchor the augmenter to the lip of the first leaflet such that the lip portion is downstream of the root portion, and/or (D) at the lip portion, between the first panel and the second panel the augmenter defines a lip opening that provides passage for blood during diastole.

Example 197. The apparatus according to example 192, wherein the central opening is shaped as a rounded square.

Example 198. The apparatus according to example 192, wherein a net area of the second panel, which is a gross area of the second panel minus an area of the central opening, is smaller than the area of the central opening.

Example 199. The apparatus according to any one of examples 169-198, wherein: (A) the augmenter further comprises a ventricular tongue extending away from the panel, and/or (B) the anchor is configured to anchor the augmenter to the lip of the first leaflet such that, at least during systole, the ventricular tongue becomes disposed on a ventricular side of the second leaflet.

Example 200. The apparatus according to any one of examples 169-199, wherein a size of the ventricular tongue is adjustable.

Example 201. The apparatus according to any one of examples 169-200, wherein the anchor is pivotably coupled to a central region of the frame, the pivotable coupling enabling the augmenter to pivot with respect to the first leaflet.

Example 202. The apparatus according to any one of examples 169-201, wherein the augmenter is configured to pivot with respect to the first leaflet responsively to fluid pressure.

Example 203. Apparatus, use with a valve of a heart, the valve having a first leaflet and a second leaflet, the first leaflet having a lip, the apparatus comprising an augmenter that comprises: (A) a frame; (B) a flexible panel attached to the frame; and/or (C) an anchor coupled to the frame at a coupling point and configured to anchor the frame to the first leaflet such that, during ventricular systole, the flexible panel billows away from the frame in response to ventricular blood pressure.

Example 204. An apparatus, comprising an augmenter for a first leaflet of a valve of a heart, the first leaflet having a lip, the valve having a second leaflet and an annulus, the valve being disposed between an atrium and a ventricle of the heart, a root of the first leaflet being attached to the annulus, the augmenter comprising: (A) a first panel; (B) a second panel, articulatably coupled to the first panel by an articulatable coupling, such that the augmenter is: (i) expandable by increasing an angular disposition between the first panel and the second panel, thereby increasing a dimension of an opening defined between the first panel and the second panel opposite the articulatable coupling, and/or (ii) collapsible by reducing the angular disposition; and/or (C) an anchor, coupled to the first panel, and configured to anchor the first panel to the first leaflet such that: (i) the first panel is disposed against the first leaflet, (ii) the articulatable coupling is disposed closer, than the lip and the opening, to the root.

Example 205. The apparatus according to example 204, wherein the augmenter is sterile.

Example 206. The apparatus according to any one of examples 204-205, wherein the anchor is configured to anchor the first panel to the first leaflet such that the augmenter expands during ventricular systole and collapses during ventricular diastole.

Example 207. The apparatus according to any one of examples 204-206, wherein the anchor is configured to anchor the first panel to the first leaflet such that the opening faces the ventricle.

Example 208. The apparatus according to any one of examples 204-207, further comprising a flexible connector connected to the first panel and to the second panel in a manner that limits expansion of the augmenter by limiting the angular disposition between the first panel and the second panel.

Example 209. The apparatus according to any one of examples 204-208, wherein the anchor is coupled to the first panel closer to the articulatable coupling than to a lip of the first panel.

Example 210. The apparatus according to any one of examples 204-209, wherein each of the first panel and the second panel is substantially circular or elliptical in shape.

Example 211. The apparatus according to any one of examples 204-210, wherein each of the first panel and the second panel is substantially rectangular in shape.

Example 212. The apparatus according to any one of examples 204-211, wherein the first panel is sufficiently flexible to conform to a shape of the first leaflet.

Example 213. The apparatus according to any one of examples 204-212, wherein the first panel is dimensioned such that the anchoring, by the anchor, of the first panel to the first leaflet is such that the first leaflet overhangs beyond the lip of the first leaflet.

Example 214. The apparatus according to any one of examples 204-213, wherein the second panel is articulatably coupled to the first panel along an entire length of an edge of each panel.

Example 215. The apparatus according to any one of examples 204-214, wherein the second panel is articulatably coupled to the first panel along a partial length of an edge of each panel.

Example 216. The apparatus according to any one of examples 204-215, wherein the articulatable coupling comprises stitches.

Example 217. The apparatus according to any one of examples 204-216, wherein the articulatable coupling comprises a hinge.

Example 218. The apparatus according to any one of examples 204-217, wherein the articulatable coupling comprises a flexure.

Example 219. The apparatus according to any one of examples 204-218, wherein the anchor comprises a clip.

Example 220. The apparatus according to any one of examples 204-219, wherein the anchor comprises a tissue-piercing element.

Example 221. The apparatus according to any one of examples 204-220, wherein the augmenter is configured such that anchoring of the first panel to the first leaflet by the anchor increases an efficiency of the valve in inhibiting regurgitation of blood through the valve during ventricular systole.

Example 222. The apparatus according to any one of examples 204-221, wherein the first panel is dimensioned such that, upon anchoring of the first panel to the first leaflet by the anchor, the first panel overhangs beyond the lip of the first leaflet.

Example 223. The apparatus according to any one of examples 204-222, wherein the second panel is dimensioned to overhang beyond an edge of the first panel.

Example 224. The apparatus according to any one of examples 204-223, wherein the first leaflet is a flailing leaflet, and wherein the augmenter is configured to be anchored to the flailing first leaflet such that the second panel coapts with the second leaflet, thereby inhibiting flailing of the first leaflet.

Example 225. The apparatus according to any one of examples 204-224, wherein the second leaflet is a flailing leaflet, and wherein the augmenter is configured to be anchored to the first leaflet such that the second panel coapts with the flailing second leaflet, thereby inhibiting flailing of the second leaflet.

Example 226. The apparatus according to any one of examples 204-225, wherein the augmenter is configured such that, while the first panel is anchored to the first leaflet by the anchor, expansion of the augmenter reduces regurgitation of blood through the valve.

Example 227. The apparatus according to any one of examples 204-226, wherein the augmenter comprises a polymer that is shaped to define the first panel and the second panel.

Example 228. The apparatus according to any one of examples 204-227, wherein the augmenter comprises a silicone that is shaped to define the first panel and the second panel.

Example 229. The apparatus according to any one of examples 204-228, further comprising a tether coupled to and extending from the first panel and configured to be attached to ventricular tissue while the anchor anchors the first panel to the first leaflet.

Example 230. The apparatus according to any one of examples 204-229, wherein the anchor is a leaflet anchor, and wherein the apparatus further comprises a ventricular anchor, coupled to the tether, and configured to anchor the tether to the ventricular tissue while the leaflet anchor anchors the first panel to the first leaflet.

Example 231. The apparatus according to any one of examples 204-229, wherein the ventricular tissue is tissue of a wall of the ventricle.

Example 232. The apparatus according to any one of examples 204-229, wherein the ventricular tissue is tissue of a papillary muscle.

Example 233. The apparatus according to any one of examples 204-229, wherein the ventricular anchor is coupled to the tether in a manner that facilitates adjustment of tension on the tether.

Example 234. The apparatus according to any one of examples 204-233, further comprising a winch coupled to the ventricular anchor, the anchor being coupled to the tether via the winch such that the tension on the tether is adjustable by actuation of the winch.

Example 235. The apparatus according to any one of examples 204-234, wherein at least the first panel comprises a cell-growth-inducing material.

Example 236. The apparatus according to any one of examples 204-235, wherein the second panel comprises a cell-growth-inducing material.

Example 237. The apparatus according to any one of examples 204-236, wherein the second panel comprises and/or is formed from a cell growth-inhibiting material.

Example 238. The apparatus according to any one of examples 204-237, wherein each of the first panel and the second panel comprise a flexible fabric.

Example 239. The apparatus according to any one of examples 204-238, wherein the augmenter further comprises at least one frame.

Example 240. The apparatus according to example 239, wherein the at least one frame serves to reinforce the fabric.

Example 241. The apparatus according to example 239, wherein the fabric of each of the first panel and the second panel is disposed over the at least one frame such that the at least one frame supports the fabric.

Example 242. The apparatus according to example 239, wherein the at least one frame is shaped to define a hoop.

Example 243. The apparatus according to example 239, wherein the at least one frame has an H shape defining a crossbar, the crossbar of the H shape traversing a face of the panel.

Example 244. The apparatus according to example 239, wherein the at least one frame is positioned along at least one edge of each panel.

Example 245. The apparatus according to example 239, wherein the at least one frame comprises a first frame and a second frame, the first frame being articulatably coupled to the second frame.

Example 246. The apparatus according to example 239, wherein the at least one frame comprises a single integrated frame, having an articulation site at which the second panel is articulatably coupled to the first panel.

Example 247. The apparatus according to example 246, wherein the single integrated frame comprises an elastic material that biases the first panel and the second panel toward assuming a predetermined angular disposition therebetween.

Example 248. The apparatus according to example 246, wherein the integrated frame comprises an elastic material that defines a flexure connection between the first panel and the second panel.

Example 249. The apparatus according to example 239, wherein the at least one frame is stitched to the fabric.

Example 250. The apparatus according to example 239, for each of the first panel and the second panel (i) the flexible fabric defines a first fabric layer, disposed over a first side of the at least one frame, and/or (ii) the panel further comprises a second fabric layer, disposed over a second side of the at least one frame, such that the frame is sandwiched between the first fabric layer and the second fabric layer.

Example 251. The apparatus according to example 239, wherein the at least one frame and the fabric are dimensioned with respect to each other such that the at least one frame and the fabric are pretensioned.

Example 252. The apparatus according to example 239, wherein when the fabric is disposed over the at least one frame, the at least one frame provides a specific form and shape to the panel.

Example 253. The apparatus according to any one of examples 204-252, wherein the augmenter is biased toward the first panel and the second panel having a predetermined angular disposition therebetween.

Example 254. The apparatus according to any one of examples 204-253, wherein the augmenter is biased to collapse by reducing the angular disposition between the second panel and the first panel.

Example 255. The apparatus according to any one of examples 204-254, wherein the augmenter is biased to expand by increasing the angular disposition between the second panel and the first panel.

Example 256. The apparatus according to any one of examples 204-255, wherein: (A) the first panel has: (i) a lip opposite the articulatable coupling, and/or (ii) a lateral edge extending between the articulatable coupling and the lip of the first panel, (B) the second panel has: (i) a lip opposite the articulatable coupling, and/or (ii) a lateral edge extending between the articulatable coupling and the lip of the second panel, and/or (C) the opening is defined by the lip of the first panel and the lip of the second panel.

Example 257. The apparatus according to any one of examples 204-256, further comprising a flexible connector connected to the first panel and to the second panel in a manner that limits expansion of the augmenter by limiting the angular disposition between the first panel and the second panel.

Example 258. The apparatus according to any one of examples 204-257, wherein the flexible connector is located opposite the articulatable coupling, extending between the first panel and the second panel.

Example 259. The apparatus according to any one of examples 204-258, wherein the flexible connector connects the lip of the first panel and the lip of the second panel.

Example 260. The apparatus according to any one of examples 204-259, wherein the flexible connector connects a substantially central part of the lip of the first panel to a substantially central part of the lip of the second panel.

Example 261. The apparatus according to any one of examples 204-260, wherein: (A) the flexible connector is a first flexible connector, (B) the augmenter further comprises a second flexible connector, (C) the first flexible connector connects a first lateral part of the lip of the first panel to a first lateral part of the lip of the second panel, and/or (iv) the second flexible connector connects: (i) a second lateral part of the lip of the first panel, substantially opposite the first lateral part of the lip of the first panel, to (ii) a second lateral part of the lip of the second panel, substantially opposite the first lateral part of the lip of the second panel.

Example 262. The apparatus according to any one of examples 204-261, wherein the flexible connector connects (A) a site on the first panel partway between the articulatable coupling and the lip of the first panel, and/or (B) a site on the second panel partway between the articulatable coupling and the lip of the second panel.

Example 263. The apparatus according to any one of examples 204-262, wherein the flexible connector connects the lateral edge of the first panel to the lateral edge of the second panel.

Example 264. The apparatus according to any one of examples 204-263, wherein the flexible connector comprises a flexible third panel extending between the first panel and the second panel.

Example 265. The apparatus according to any one of examples 204-264, wherein the flexible connector includes a cell-growth-inhibiting substance.

Example 266. The apparatus according to any one of examples 204-265, wherein the flexible connector includes a cell-growth-promoting substance.

Example 267. The apparatus according to any one of examples 204-266, wherein the flexible third panel connects the lip of the first panel to the lip of the second panel.

Example 268. The apparatus according to any one of examples 204-267, wherein the third panel comprises a fabric.

Example 269. The apparatus according to any one of examples 204-268, wherein the third panel comprises a net.

Example 270. The apparatus according to any one of examples 204-269, wherein the anchor is configured to anchor the first panel to the first leaflet such that the third panel coapts with the second leaflet during systole.

Example 271. The apparatus according to any one of examples 204-270, wherein the anchor is configured to anchor the first panel to the first leaflet such that the third panel faces the ventricle during systole.

Example 272. The apparatus according to any one of examples 204-271, wherein: (A) the lateral edge of the first panel is a first lateral edge of the first panel, (B) the lateral edge of the second panel is a first lateral edge of the second panel, substantially aligned with the first lateral edge of the first panel, (C) the first panel has a second lateral edge, substantially opposite the first lateral edge of the first panel, and extending between the articulatable coupling and the lip of the first panel, (D) the second panel has a second lateral edge substantially opposite the first lateral edge of the second panel, extending between the articulatable coupling and the lip of the second panel, and substantially aligned with the second lateral edge of the first panel, (E) the third panel extends between the first lateral edge of the first panel and the first lateral edge of the second panel, and/or (F) the augmenter further comprises a flexible fourth panel, extending between the second lateral edge of the first panel and the second lateral edge of the second panel.

Example 273. The apparatus according to any one of examples 204-272, wherein the third panel and the fourth panel comprise folds configured to collapse inward when the opening collapses.

Example 274. The apparatus according to any one of examples 204-273, wherein the flexible connector comprises at least one connector element selected from: a wire, and a thread.

Example 275. The apparatus according to any one of examples 204-274, wherein the flexible connector comprises exactly one connector element.

Example 276. The apparatus according to any one of examples 204-275, wherein the anchor is coupled to the first panel closer to the articulatable coupling than to the lip of the first panel.

Example 277. The apparatus according to any one of examples 204-276, wherein the anchor comprises a toggle anchor.

Example 278. The apparatus according to any one of examples 204-277, wherein the toggle anchor has a blunt end and a sharp end and is configured to be advanced through the first leaflet sharp-end-first.

Example 279. The apparatus according to any one of examples 204-278, wherein the anchor is coupled to the first panel by a flexible thread.

Example 280. The apparatus according to any one of examples 204-279, wherein the anchor is a first anchor, and wherein the augmenter further comprises a second anchor that is coupled to the first panel, and that is configured to anchor the first panel to the first leaflet.

Example 281. The apparatus according to example 280, wherein the first anchor comprises a first toggle anchor, and the second anchor comprises a second toggle anchor.

Example 282. The apparatus according to any one of examples 204-281, wherein the augmenter is configured such that, while (i) the first panel is anchored to the first leaflet, and/or (ii) the articulatable coupling is disposed closer to the root, than the lip and the opening, blood flow in the heart transitions the augmenter toward its expanded configuration during ventricular systole, and toward its collapsed configuration during ventricular diastole.

Example 283. The apparatus according to any one of examples 204-282, further comprising a flexible connector connected to the first panel and to the second panel in a manner that limits expansion of the augmenter during ventricular systole.

Example 284. An apparatus, comprising an augmenter for a first leaflet of a valve of a heart of a subject, the first leaflet having a lip, the valve being disposed between an atrium and a ventricle of the heart and having a second leaflet, the apparatus comprising: (A) a first panel; (B) a second panel; (C) a third panel; and/or (D) an anchor, wherein: (i) each of the panels: (1) has a respective outer edge, (2) extends inwardly from the outer edge, such that the first, second, and third panels converge at an axis of the augmenter, and/or (3) defines a panel plane that is substantially parallel with the axis, and/or (ii) the anchor is configured to anchor the first panel to the first leaflet.

Example 285. The apparatus according to example 284, wherein the augmenter is sterile.

Example 286. The apparatus according to any one of examples 284-285, wherein the augmenter comprises a polymer that is shaped to define the first panel, the second panel, and the third panel.

Example 287. The apparatus according to any one of examples 284-286, wherein the augmenter comprises a silicone that is shaped to define the first panel, the second panel, and the third panel.

Example 288. The apparatus according to any one of examples 284-287, wherein the anchor is configured to attach the first panel to the first leaflet such that the second panel extends away from the first panel toward the second leaflet.

Example 289. The apparatus according to any one of examples 284-288, wherein the anchor is configured to anchor the first panel to the first leaflet such that the third panel extends away from the first panel toward the ventricle.

Example 290. The apparatus according to any one of examples 284-289, wherein the anchor is coupled to the first panel closer to the outer edge of the first panel than to the axis of the augmenter.

Example 291. The apparatus according to any one of examples 284-290, wherein each of the first panel, the second panel, and the third panel is substantially circular or elliptical in shape.

Example 292. The apparatus according to any one of examples 284-291, wherein each of the first panel, the second panel, and the third panel is substantially rectangular in shape.

Example 293. The apparatus according to any one of examples 284-292, wherein the first panel is sufficiently flexible to conform to a shape of the first leaflet.

Example 294. The apparatus according to any one of examples 284-293, wherein the first panel is dimensioned such that the anchoring, by the anchor, of the first panel to the first leaflet is such that the third panel overhangs beyond the lip of the first leaflet.

Example 295. The apparatus according to any one of examples 284-294, wherein: (A) the second panel and the third panel collectively define a face, and/or (B) the anchor is configured to anchor the first panel to the first leaflet such that: (i) the first panel is disposed against the first leaflet, and/or (ii) the face is presented as a coaptation surface to the second leaflet.

Example 296. The apparatus according to any one of examples 284-295, wherein the anchor is configured to anchor the first panel to the first leaflet such that the third panel is disposed in the ventricle.

Example 297. The apparatus according to any one of examples 284-296, wherein the anchor is configured to anchor the first panel to the first leaflet such that, during ventricular systole, the second panel extends, within the atrium, away from the first panel and across an upstream surface of the second leaflet.

Example 298. The apparatus according to any one of examples 284-297, wherein the anchor is configured to anchor the first panel to the first leaflet such that, during ventricular systole, the second panel is disposed against an upstream surface of the second leaflet.

Example 299. The apparatus according to any one of examples 284-298, wherein the anchor is configured to anchor the first panel to the first leaflet such that, during ventricular systole, a lip of the second leaflet contacts the third panel.

Example 300. The apparatus according to any one of examples 284-299, further comprising a tether coupled to and extending from the first panel and configured to tether the first panel to ventricular tissue of the heart.

Example 301. The apparatus according to any one of examples 284-300, wherein the anchor is a leaflet anchor, and wherein the apparatus further comprises a ventricular anchor, coupled to the tether, and configured to anchor the tether to the ventricular tissue while the leaflet anchor anchors the first panel to the first leaflet.

Example 302. The apparatus according to any one of examples 284-301, wherein the ventricular tissue is tissue of a wall of the ventricle.

Example 303. The apparatus according to any one of examples 284-302, wherein the ventricular tissue is tissue of a papillary muscle.

Example 304. The apparatus according to any one of examples 284-303, wherein the ventricular anchor is coupled to the tether in a manner that facilitates adjustment of tension on the tether.

Example 305. The apparatus according to any one of examples 284-304, further comprising a winch coupled to the ventricular anchor, the anchor being coupled to the tether via the winch such that the tension on the tether is adjustable by actuation of the winch.

Example 306. The apparatus according to any one of examples 284-305, wherein at least the first panel comprises a cell-growth-inducing material.

Example 307. The apparatus according to any one of examples 284-306, wherein the second panel includes a cell-growth-inducing material.

Example 308. The apparatus according to any one of examples 284-307, wherein the third panel includes a cell growth-inducing material.

Example 309. The apparatus according to any one of examples 284-308, wherein the second panel includes a cell-growth-inhibiting material.

Example 310. The apparatus according to any one of examples 284-309, wherein the third panel includes a cell growth-inhibiting material.

Example 311. The apparatus according to any one of examples 284-310, wherein each of the first panel, the second panel, and the third panel comprises a flexible fabric.

Example 312. The apparatus according to any one of examples 284-311, wherein the augmenter further comprises a frame.

Example 313. The apparatus according to any one of examples 284-312, wherein the frame serves to reinforce the fabric.

Example 314. The apparatus according to any one of examples 284-313, wherein the fabric of each of the first panel, the second panel, and the third panel is disposed over the frame such that the frame supports the fabric.

Example 315. The apparatus according to any one of examples 284-314, wherein, for each of the first, second, and third panels, the frame extends along the outer edge the panel, substantially parallel with the axis.

Example 316. The apparatus according to any one of examples 284-315, wherein the frame comprises a single integrated frame defining an outline of the first panel, the second panel, and the third panel.

Example 317. The apparatus according to any one of examples 284-316, wherein the integrated frame comprises an elastic material that biases the first panel, the second panel, and the third panel toward assuming a predetermined angular disposition with respect to each other.

Example 318. The apparatus according to any one of examples 284-317, wherein the integrated frame comprises an elastic material.

Example 319. The apparatus according to any one of examples 284-318, wherein the frame is stitched to the fabric.

Example 320. The apparatus according to any one of examples 284-319, wherein, for each of the first panel, the second panel, and the third panel: (i) the flexible fabric defines a first fabric layer, disposed over a first side of the frame, and/or (ii) the panel further comprises a second fabric layer, disposed over a second side of the frame, such that the frame is sandwiched between the first fabric layer and the second fabric layer.

Example 321. The apparatus according to any one of examples 284-320, wherein the frame and the fabric are sized with respect to each other such that the frame and the fabric are pretensioned.

Example 322. The apparatus according to any one of examples 284-321, wherein when the fabric is disposed over the frame, the frame provides a specific form and shape to the first panel, the second panel, and the third panel.

Example 323. A method useable with a valve of a heart of a subject, the valve having a first leaflet and a second leaflet, and being disposed between an atrium and a ventricle of the heart, the method comprising: (A) transluminally advancing an augmenter to the heart, the augmenter including: (i) a first panel, (ii) a second panel articulatably coupled to the first panel, and/or (iii) an anchor; and/or (B) anchoring the first panel to the first leaflet using the anchor, such that: (i) the first panel of the augmenter is disposed against the first leaflet, and/or (ii) the second panel, in response to blood flow in the heart: (1) deflects away from the first panel and toward the second leaflet during ventricular systole; and/or (2) deflects away from the second leaflet and toward the first panel during ventricular diastole.

Example 324. The method according to example 323, further comprising sterilizing the augmenter.

Example 325. The method according to any one of examples 323-324, wherein anchoring the first panel to the first leaflet comprises anchoring the first panel to the first leaflet such that the second panel, in response to blood flow in the heart, coapts with the second leaflet during ventricular systole, thereby reducing regurgitation through the valve.

Example 326. The method according to any one of examples 323-325, wherein deflection of the second panel during ventricular systole leads to the second panel coapting with the second leaflet.

Example 327. The method according to any one of examples 323-326, further comprising tethering the first panel to ventricular tissue using a tether connected to the first panel.

Example 328. The method according to any one of examples 323-327, further comprising adjusting tension on the tether.

Example 329. The method according to any one of examples 323-328, wherein

tethering the first panel to the ventricular tissue comprises anchoring the tether to the ventricular tissue using a tissue anchor attached to the tether.

Example 330. The method according to any one of examples 323-329, further comprising coupling the tissue anchor to the tether via a winch.

Example 331. The method according to any one of examples 323-330, further comprising adjusting tension on the tether by actuating the winch.

Example 332. The method according to any one of examples 323-331, further comprising attaching a flexible third panel between the first panel and the second panel.

Example 333. The method according to any one of examples 323-332, wherein deflection of the second panel during ventricular systole is accompanied by the flexible third panel coapting with the second leaflet.

Example 334. A method useable with a valve of a heart of a subject, the valve having a first leaflet and a second leaflet, and being disposed between an atrium and a ventricle of the heart, the method comprising: (A) transluminally advancing an augmenter to the heart, the augmenter including: (i) a first panel, (ii) a second panel, (iii) a third panel, and/or (iv) an anchor; and/or (B) anchoring the first panel to the first leaflet, wherein each of the panels: (i) has a respective outer edge, (ii) extends inwardly from the outer edge, such that the first, second, and third panels converge at an axis of the augmenter, and/or (iii) defines a respective panel plane that is substantially parallel with the axis.

Example 335. The method according to example 334, further comprising sterilizing the augmenter.

Example 336. The method according to any one of examples 334-335, wherein: (i) anchoring the first panel to the first leaflet comprises anchoring the first panel to the first leaflet while the second panel and the third panel are disposed within a delivery tool, and/or (ii) the method further comprises subsequently deploying the second panel and the third panel from the delivery tool.

Example 337. The method according to example 336, wherein deploying the second panel and the third panel from the delivery tool comprises deploying the second panel and the third panel from the delivery tool such that the second panel and the third panel deflect away from each other.

Example 338. Apparatus, comprising a leaflet augmenter useable at a valve of a heart of a subject, the valve having at least a first leaflet and a second leaflet, the apparatus comprising: (i) a first panel, and a second panel articulatably connected to the first panel; and/or (ii) a tissue-piercing element coupled to the first panel and configured to anchor the first panel to the first leaflet such that, during ventricular systole, closure of the valve is accompanied by articulation of the second panel away from the first panel and toward the second leaflet.

Example 339. A method for manufacturing an augmenter for a valve of a heart, the valve having a first leaflet and a second leaflet, the method comprising: (A) cutting, from a shape memory alloy, a unitary skeleton that includes a frame-region and an anchor-region; (B) in a first heat-setting procedure, heat-setting the anchor-region to define an anchor; (C) in a second heat-setting procedure, heat-setting the frame-region to define a frame; and/or (D) forming a panel by attaching a sheet to the frame, the anchor being configured to anchor the augmenter to the first leaflet such that the panel coapts with the second leaflet during systole.

Example 340. The method according to example 339, further comprising sterilizing the augmenter.

Example 341. The method according to any one of examples 339-340, wherein heat-setting the anchor-region comprises heat-setting the anchor-region using a first mold, and heat-setting the frame-region comprises heat-setting the frame-region using a second mold.

Example 342. The method according to any one of examples 339-341, wherein the anchor comprises a clip, and heat-setting the anchor-region to define the anchor comprises heat-setting the anchor-region to define the clip.

Example 343. The method according to any one of examples 339-342, wherein the method comprises performing the second heat-setting procedure subsequently to performing the first heat-setting procedure.

Example 344. The method according to any one of examples 339-343, wherein the method comprises constraining the anchor-region in a heat-set shape during the second heat-setting procedure.

Example 345. The method according to any one of examples 339-344, wherein the cutting is performed by a laser.

Example 346. The method according to any one of examples 339-345, wherein the method further comprises anchoring the augmenter to the valve by clamping the anchor to the first leaflet of the valve.

Example 347. The method according to any one of examples 339-346, wherein a first heat-setting mold is configured to mold the anchor-region, such that providing the frame and anchor with a first shape memory comprises using the first heat-setting mold to mold the anchor-region of the shape memory alloy to a clamp shape.

Example 348. The method according to any one of examples 339-347, wherein a second heat-setting mold is configured to provide the frame with a curvature, such that that providing the frame with a second shape memory comprises using the second heat-setting mold to provide the frame with a curvature that approximates that of the second leaflet.

Example 349. Apparatus, comprising an augmenter for a valve of a heart, the valve having a first leaflet and a second leaflet, and the augmenter comprising: (i) a unitary skeleton, cut from a shape-memory alloy to define a frame-region and an anchor-region, the frame-region being heat-set to define a frame, and the anchor-region being heat-set to define an anchor; and/or (ii) a sheet, attached to the frame in a manner that defines a panel, the anchor being configured to anchor the augmenter to the first leaflet such that the panel coapts with the second leaflet during systole.

Example 350. A method useable with a valve of a heart of a subject, the valve having a first leaflet and a second leaflet, and being disposed between an atrium and a ventricle of the heart, the method comprising: (A) transluminally advancing an augmenter to the heart, the augmenter including: (i) a flexible pouch, having a downstream opening and an external surface, the pouch coupled to an anchor; (B) anchoring the anchor to the first leaflet, such that the pouch: (i) is anchored to the lip of the first leaflet by the anchor being anchored to the first leaflet, and/or (ii) responsively to ventricular systole, inflates via the downstream opening such that the external surface coapts against the opposing leaflet.

Example 351. The method according to example 350, wherein the method further comprises using an imaging modality to view coaptation of the external surface against the opposing leaflet.

Example 352. The method according to any one of examples 350-351, further comprising sterilizing the augmenter.

Example 353. The method according to any one of examples 350-352, wherein the augmenter further includes an adjustable strap coupled to the pouch, and the method further comprises adjusting a length of the strap to adjust an internal dimension of the pouch.

Example 354. The method according to example 353, wherein adjusting the length of the strap to adjust the internal dimension of the pouch comprises shortening the strap to decrease the internal dimension of the pouch.

Example 355. The method according to example 353, wherein adjusting the length of the strap to adjust the internal dimension of the pouch comprises lengthening the strap to increase the internal dimension of the pouch.

Example 356. The method according to example 353, wherein adjusting the length of the strap comprises adjusting the length of the strap while viewing the pouch in the heart using an imaging modality.

Example 357. The method according to example 353, wherein: (A) the adjustable strap is a first adjustable strap, (B) adjusting the length of the adjustable strap comprises adjusting the length of the first adjustable strap, (C) the augmenter further includes a second adjustable strap coupled to the pouch, and/or (D) the method further comprises adjusting a length of the second adjustable strap to adjust the internal dimension of the pouch.

Example 358. The method according to any one of examples 350-357, wherein: the opposing leaflet is a first opposing leaflet, the valve is a tricuspid valve having a second opposing leaflet, and anchoring the anchor to the first leaflet comprises anchoring the anchor to the first leaflet such that, responsively to ventricular systole, the pouch inflates via the downstream opening, such that the external surface coapts against both the first opposing leaflet and the second opposing leaflet.

Example 359. The method according to example 358, wherein anchoring the anchor to the first leaflet comprises anchoring the anchor to the first leaflet such that, responsively to ventricular systole, at least part of the pouch interposes between the first opposing leaflet and the second opposing leaflet.

Example 360. The method according to example 358, wherein the augmenter further includes an adjustable strap coupled to the pouch, and the method further comprises adjusting a length of the adjustable strap to adjust an internal dimension of the pouch.

Example 361. The method according to example 360, wherein adjusting the length of the adjustable strap to adjust the internal dimension of the pouch comprises shortening the adjustable strap to decrease the internal dimension of the pouch.

Example 362. The method according to example 360, wherein adjusting the length of the adjustable strap to adjust the internal dimension of the pouch comprises lengthening the adjustable strap to increase the internal dimension of the pouch.

Example 363. The method according to example 360, wherein adjusting the length of the strap comprises adjusting the length of the adjustable strap while viewing the pouch in the heart using an imaging modality.

Example 364. The method according to example 360, wherein: (A) the adjustable strap having a length is a first adjustable strap having a first length, (B) adjusting the length of the adjustable strap comprises adjusting the first length, (C) the augmenter further includes a second adjustable strap coupled to the pouch and having a second length, and/or (D) the method further comprises adjusting the second length to adjust the internal dimension of the pouch.

Example 365. The method according to example 364, wherein adjusting the first length and the second length is performed in a manner that, responsively to ventricular systole, at least part of the pouch interposes between the first opposing leaflet and the second opposing leaflet.

Example 366. The method according to example 364, wherein the method further comprises using an imaging modality to view coaptation of the external surface against the first opposing leaflet and the second opposing leaflet.

Example 367. The method according to example 366, wherein using the imaging modality to view coaptation of the external surface against the first opposing leaflet and the second opposing leaflet is performed while adjusting the first adjustable strap and the second adjustable strap.

Although the operations of some of the disclosed methods are described in a particular, sequential order for convenient presentation, it should be understood that this manner of description encompasses rearrangement, unless a particular ordering is required by specific language set forth below. For example, operations described sequentially can in some cases be rearranged or performed concurrently. Moreover, for the sake of simplicity, the attached figures may not show the various ways in which the disclosed systems, apparatuses, devices, methods, etc. can be used in conjunction with other systems, apparatuses, devices, methods, etc.

The present disclosure is not limited to the examples that have been particularly shown and described hereinabove. Rather, the scope of the present disclosure includes both combinations and subcombinations of the various features described hereinabove, as well as variations and modifications thereof that are not in the prior art, which would occur to persons skilled in the art upon reading the foregoing description.

For instance, to the extent that the systems, assemblies, and/or apparatuses disclosed herein are described as being associated with a particular valve (e.g., mitral and/or tricuspid) and/or are described as being anchored to a particular leaflet (e.g., septal, anterior, posterior), a skilled artisan will understand that implementations of the present disclosure are not limited to the specific examples described herein. For instance, implementations of the present disclosure can be used with the mitral and tricuspid valves and may be anchored to any of the leaflets associated therewith (e.g., septal, anterior, and/or posterior).

What is claimed is:

1. An apparatus, useable with a valve disposed between an atrium and a ventricle of a heart, the valve having a first leaflet and an opposing leaflet, the first leaflet having a lip, the apparatus comprising an augmenter that comprises:
a flexible pouch, having an external surface, an interior, and a downstream opening that provide fluid communication between the interior and an outside of the pouch; and
an anchor, coupled to the pouch, and configured to anchor the pouch to the lip of the first leaflet by the anchor being anchored to the first leaflet, such that:
the downstream opening opens into the ventricle; and
responsively to ventricular systole:
the pouch inflates via the downstream opening; and
the external surface coapts against the opposing leaflet.

2. The apparatus according to claim 1, wherein:
the pouch is shaped to define a vent at an upstream region of the pouch; and
the vent configured to allow limited retrograde blood flow therethrough.

3. The apparatus according to claim 2, wherein the vent is one of multiple vents at a proximal end of the pouch.

4. The apparatus according to claim 3, wherein the multiple vents are disposed on an opposite side of the pouch to the anchor.

5. The apparatus according to claim 2, wherein the pouch is conical and defines an apex, the vent being disposed at the apex.

6. The apparatus according to claim 2, wherein the vent comprises a grommet that delimits the vent.

7. The apparatus according to claim 2, wherein the vent comprises a check valve that is oriented to allow the limited retrograde blood flow through the vent and to inhibit antegrade blood flow through the vent.

8. The apparatus according to claim 7, wherein the check valve is a duckbill valve.

9. The apparatus according to claim 7, wherein the check valve is a reed valve.

10. The apparatus according to claim 7, wherein the check valve is a diaphragm valve.

11. The apparatus according to claim 1, wherein:
the pouch is formed from a fabric; and
the pouch comprises at least one wire that:
has a stiffness greater than that of the fabric; and
extends, in association with the fabric, from an apical vent toward the downstream opening.

12. The apparatus according to claim 11, wherein the at least one wire includes at least three wires, distributed circumferentially around the pouch, such that the at least three wires deflect away from each other as the pouch inflates.

13. The apparatus according to claim 1, further comprising a stem that couples the pouch to the anchor by extending:
from the anchor, outside of the pouch toward the downstream opening;
under a rim of the downstream opening, thereby defining a bight of the stem; and
from the bight, through the interior to an upstream apex of the pouch, thereby defining a mast of the augmenter.

14. The apparatus according to claim 1, wherein the augmenter comprises an adjustment mechanism that is actuatable to adjust positioning between the anchor and the pouch.

15. The apparatus according to claim 1, wherein the augmenter is configured to facilitate adjustment of positioning between the anchor and the pouch while the pouch remains anchored to the lip by the anchor, in a manner that changes a proportion of the pouch that is disposed within the atrium during ventricular systole.

16. The apparatus according to claim 1, further comprising a stem via which the pouch is adjustably coupled to the anchor.

17. The apparatus according to claim 1, further comprising a resilient wire, coupled to an upstream part of the pouch in a manner that squeezes blood out of the upstream part of the pouch during diastole.

18. The apparatus according to claim 1, wherein the augmenter further comprises an adjustable strap coupled to the pouch such that adjusting a length of the strap adjusts an internal dimension of the pouch.

19. The apparatus according to claim 1, wherein the augmenter further comprises:
a frame to which the pouch and the anchor are coupled; and
a stabilizer, coupled to the frame, extending away from the frame to a projection of the stabilizer, the stabilizer being configured to be positioned with the projection at a site in the heart in a manner that resists upstream pivoting of the pouch.

20. The apparatus according to claim 19, wherein the projection comprises a foil that has a shape and an angle of attack that generates lift in a manner that resists the upstream pivoting.
